(19) United States
(12) Patent Application Publication
(10) Pub. No.: US 2020/0113902 A1
(43) Pub. Date: Apr. 16, 2020

(54) COMPOSITIONS AND METHODS FOR TREATING CANCERS WITH COVALENT INHIBITORS OF CYCLIN-DEPENDENT KINASE 7 (CDK7)

(71) Applicant: Syros Pharmaceuticals, Inc., Cambridge, MA (US)

(72) Inventors: Nisha Rajagopal, Boston, MA (US); Graeme Hodgson, Boxborough, MA (US); Emmanuelle Di Tomaso, Lexington, MA (US); Liv Helena Johannessen, Cambridge, MA (US); Shanhu Hu, Medford, MA (US)

(21) Appl. No.: 16/621,318

(22) PCT Filed: Jun. 12, 2018
(86) PCT No.: PCT/US2018/037147

§ 371 Date: Dec. 11, 2019

Related U.S. Application Data
(60) Provisional application No. 62/518,429, filed on Jun. 12, 2017.
(60) Provisional application No. 62/539,912, filed on Aug. 1, 2017.
(60) Provisional application No. 62/578,157, filed on Oct. 27, 2017.
(60) Provisional application No. 62/593,734, filed on Dec. 1, 2017.
(60) Provisional application No. 62/641,638, filed on Mar. 12, 2018.

(51) Int. Cl. A61K 31/506; C12Q 1/6886; A61P 35/00

(52) U.S. Cl. CPC A61K 31/506; A61K 45/06; A61P 35/00; C12Q 1/6886

(57) ABSTRACT

The present invention relates to methods of identifying subjects suffering from various types of cancer who are more likely to respond to treatment with a covalent CDK7 inhibitor, such as N-((1S,3R)-3-(5-chloro-4-(1H-indol-3-yl)pyrimidin-2-ylamino)-1-methylcyclohexyl)-5-((E)-4-(dimethylamino)but-2-enamido)picolinamide (Compound 1), either alone or in combination with other classes of anti-cancer therapies based on the presence or absence of certain biomarkers. In addition, the present invention relates to combinations of Compound 1 and one or more other anti-cancer therapies, kits containing them, and the use of such combinations in treating subjects suffering from various types of cancers.

### CROSS-REFERENCE TO RELATED APPLICATIONS

This application is a U.S. National Phase Application under 35 U.S.C. § 371 of International Application No. PCT/US2018/037147, filed Jun. 12, 2018, which claims the benefit of the filing dates of U.S. provisional application No. 62/641,638, filed Mar. 12, 2018, U.S. provisional application No. 62/593,734, filed Dec. 1, 2017, U.S. provisional application No. 62/578,157, filed Oct. 27, 2017, U.S. provisional application No. 62/539,912, filed Aug. 1, 2017, and U.S. provisional application No. 62/518,429, filed Jun. 12, 2017. The entire content of each of these applications is hereby incorporated by reference herein in its entirety.

### BACKGROUND OF THE INVENTION

The transcriptional kinase CDK7 (cyclin-dependent kinase 7) has been implicated in the pathogenesis of multiple malignancies, including leukemias (e.g., acute myeloid leukemia (AML)), breast cancer (e.g., triple negative breast cancer (TNBC)), and ovarian cancer, and it may play important roles in regulation of oncogenic transcriptional dependencies and in regulation of the mitochondrial apoptosis machinery in tumors. For example, data from recent studies have demonstrated that TNBC cells are highly dependent on the transcriptional regulator CDK7 and suggest that the mitochondrial apoptosis pathway is important in mediating cell survival in CDK7-dependent cells. Further, TNBC has been shown to have a distinct epigenetic and transcriptional program, with super-enhancers (SEs) mediating the expression of key oncogenic drivers such as MYC.

### SUMMARY OF THE INVENTION

The present invention features, inter alia, compositions and methods for identifying or selecting cancer patients who are likely to respond well to treatment with a covalent CDK7 inhibitor (i.e., diagnostic methods) and/or methods for treating such patients with a covalent inhibitor of CDK7, either alone or in combination with other classes of anti-cancer therapeutics, as described further below. The diagnostic methods include a step of identifying or selecting a subject suffering from a cancer that is likely to respond well to treatment with a covalent CDK7 inhibitor, such as N-((1S,3R)-3-(5-chloro-4-(1H-indol-3-yl)pyrimidin-2-ylamino)-1-methylcyclohexyl)-5-((E)-4-(dimethylamino)but-2-enamido)picolinamide (Compound 1), or pharmaceutically acceptable salts thereof, and the treatment methods include a step of administering a covalent CDK7 inhibitor to an identified or selected subject. Thus, methods in which a patient is only diagnosed as being a suitable (or good) candidate for treatment, methods in which a selected patient within an identified subset of patients is only treated as described herein, and methods in which a patient is both diagnosed and treated as described herein are encompassed by the present invention.

For ease of reading, we will not refer to both compounds that are covalent CDK7 inhibitors (e.g., Compound 1) and pharmaceutically acceptable salts thereof when describing each and every composition, method, and use within the scope of the invention. It is to be understood that where a covalent CDK7 inhibitor described herein can be used, a pharmaceutically acceptable salt thereof may also be useful, and that determination (determining an appropriate salt form of a compound) is well within the ability of one of ordinary skill in the art.

The diagnostic methods that identify or select a subject for treatment can include a step of analyzing one or more biomarkers in a biological sample obtained from the subject by determining, having determined, or receiving information concerning the state of one or more specific biomarkers (e.g., the presence, absence, or copy number of a biomarker gene in wild type or mutant form, the association of a biomarker gene with a super-enhancer (SE) or a SE of a certain strength, the level of expression of the biomarker gene (as evidenced, for example, by mRNA levels) and/or the level of expression or activity of the protein encoded by the biomarker gene). The state of a biomarker can be assessed in terms of any one or more of the features just listed regardless of the precise method or context in which the biomarker is being assessed. The state of a given biomarker may be equal to or above a pre-determined threshold level or equal to or below a pre-determined threshold level. In the methods of the present invention, one can analyze a biomarker selected from MYC, CDK18, CDK19, CCNE1, FGFR1, or certain E2F pathway members by determining, having determined, and/or receiving information that the state of such a biomarker is equal to or above (e.g., above) a pre-determined threshold level. Alternatively, or in addition, one can analyze a biomarker selected from BCLXL, CDK7, CDK9, and RB1 (or certain E2F pathway members) by determining, having determined, and/or receiving information that the state of such biomarker is equal to or below (e.g., below) a pre-determined threshold level. The choice of which biomarker(s) to utilize may depend, in part, on the particular cancer that the subject is suffering from, as well as other factors described herein.

The compositions of the invention include pharmaceutically acceptable compositions that include combinations of a covalent CDK7 inhibitor, as described herein (e.g., Compound 1 or a pharmaceutically acceptable salt thereof), and one or more other anti-cancer therapeutics (as described herein). In keeping with convention, in any embodiment requiring these two agents, we may refer to the covalent CDK7 inhibitor or a pharmaceutically acceptable salt thereof as the “first” active agent and to the other anti-cancer therapeutic as the “second” active agent. In case of any doubt, the first and second agents are distinct from one another. The compositions of the invention also include kits that include a covalent CDK7 inhibitor and instructional materials that describe a suitable patient, methods of identifying a suitable patient for treatment (e.g., by any one of the diagnostic stratification methods described herein or assessment of resistance to a previously administered anti-cancer agent), and/or instructions for administering the covalent CDK7 inhibitor in combination with at least one other anti-cancer therapy or therapeutic. The kits of the invention can also include a second anti-cancer agent, including any one or more of the second agents described herein.

Each therapeutic method and any diagnostic method that employs a covalent CDK7 inhibitor may also be expressed in terms of use and vice versa. For example, the invention encompasses the use of a compound or composition described herein for the treatment of a disease described herein (e.g., cancer); a compound or composition for use in diagnosing and/or treating or a disease (e.g., cancer); and the use of the compound or composition for the preparation of a medicament for treating a disease described herein (e.g., cancer).

The methods of the invention that concern diagnosing and/or treating a disease described herein (e.g., a cancer (or use of a covalent CDK7 inhibitor for such purposes)) may specifically exclude any one or more of the types of cancers described herein. For example, the invention features methods of treating cancer by administering a compound as described herein (e.g., a compound of Formula A (e.g., Compound 1)) with the proviso that the cancer is not a breast cancer; with the proviso that the cancer is not a breast cancer or a leukemia; with the proviso that the cancer is not a breast cancer, a leukemia, or an ovarian cancer; and so forth, with exclusions selected from any of the diseases listed herein and with the same notion of variable exclusion from lists of elements relevant to other aspects of the invention (e.g., chemical substituents of a compound described herein or components of kits and pharmaceutical compositions).

In one aspect, the invention features the use of a covalent CDK7 inhibitor described herein, e.g., Compound 1

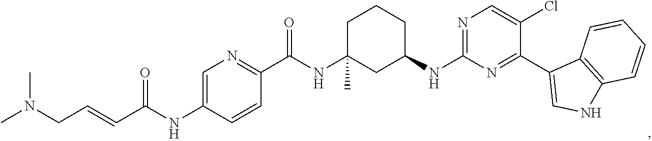 [SMILES: CN(C)C/C=C/C(=O)Nc1ccc(C(=O)N[C@@]2(C)CCC[C@@H](Nc3ncc(Cl)c(-c4c[nH]c5ccccc45)n3)C2)nc1]

or a pharmaceutically acceptable salt thereof, in treating cancer in a selected patient, wherein the patient is selected by virtue of: (a) having a level of B-cell lymphoma-extra large (BCLXL) mRNA in the cancer equal to or below a pre-determined threshold; and/or (b) having in at least one of the genes involved in the RB-E2F pathway an alteration in the DNA, an epigenetic alteration, or an alteration in the level of expression of mRNA or protein; and/or (c) being treated with a platinum-based therapeutic agent (e.g., carboplatin or oxaliplatin) or whose cancer has developed resistance to a platinum-based therapeutic agent (e.g., carboplatin or oxaliplatin); and/or (d) having become or at risk of becoming resistant to treatment with a CDK4/6 inhibitor when used alone or in combination with one or more of an aromatase inhibitor, a selective estrogen receptor modulator or a selective estrogen receptor degrader. In the context of this use, the cancer can be a triple negative breast cancer (TNBC), ovarian cancer, non-small cell lung cancer, or acute myeloid leukemia (AML) and the patient has been selected by virtue of having a level of BCLXL mRNA in the cancer equal to or below the pre-determined threshold level. The patient can be one who has undergone, is presently undergoing, or is intending to undergo treatment with a Bcl-2 inhibitor, such as venetoclax. In the context of this use, the patient can be selected by virtue of having one or more of: a) a level of CCNE1 gene copy number, mRNA or protein in the cancer equal to or above a pre-determined threshold; b) a level of RB1 gene copy number, mRNA or protein in the cancer equal to or below a pre-determined threshold, or an absence of an expressed wild-type RB1 gene; c) a level of CDK6 mRNA equal to or above a pre-determined threshold level; d) a level of CCND2 mRNA equal to or above a pre-determined threshold level; or e) a level of CDKN2A mRNA equal to or below a pre-determined threshold level. In specific embodiments, the patient is selected by virtue of having a level of CCNE1 gene copy number, mRNA or protein in the cancer equal to or above a pre-determined threshold; a level of RB1 gene copy number, mRNA or protein in the cancer equal to or below a pre-determined threshold; or an absence of an expressed wild-type RB1 gene. In the context of this use, the patient can be suffering from ovarian cancer, breast cancer, TNBC, or hormone receptor-positive breast cancer, and the patient may be one who has undergone, is presently undergoing, or is intending to undergo treatment with a selective estrogen receptor modulator such as tamoxifen, a selective estrogen receptor degrader such as fulvestrant, and/or a PARP inhibitor, such as olaparib or niraparib.

In another aspect, the invention features the use of a covalent CDK7 inhibitor described herein, e.g., Compound 1

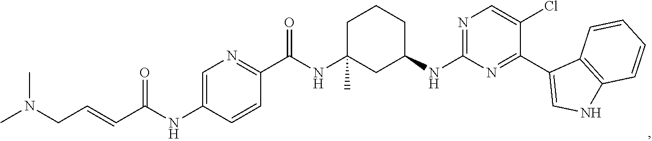 [SMILES: CN(C)C/C=C/C(=O)Nc1ccc(C(=O)N[C@@]2(C)CCC[C@@H](Nc3ncc(Cl)c(-c4c[nH]c5ccccc45)n3)C2)nc1]

or a pharmaceutically acceptable salt thereof, in a combination therapy with an effective amount of a second agent in treating a patient who has cancer, wherein: (a) the cancer is TNBC, an estrogen receptor-positive (ER⁺) breast cancer, pancreatic cancer, or a squamous cell cancer of the head or neck; and the second agent is a CDK4/6 inhibitor; (b) the cancer is a breast cancer, or an ovarian cancer; and the second agent is a PARP inhibitor; (c) the cancer is AML; and the second agent is a FLT3 inhibitor; (d) the cancer is an ovarian cancer; and the second agent is a platinum-based anti-cancer agent; (e) the cancer is TNBC, AML, Ewing's sarcoma, or an osteosarcoma; and the second agent is a BET inhibitor; (f) the cancer is TNBC, AML, an ovarian cancer, or non-small cell lung cancer; and the second agent is a Bcl-2 inhibitor. In particular embodiments, the cancer is AML and the second agent is a Bcl-2 inhibitor, such as venetoclax; the cancer is an epithelial ovarian cancer, a fallopian tube cancer, a primary peritoneal cancer, a triple negative breast cancer or a Her2⁺/ER⁻/PR⁻ breast cancer and the second agent is a PARP inhibitor, such as olaparib or niraparib; the cancer is an ovarian cancer and the second agent is a platinum-based anti-cancer agent, such as carboplatin or oxaliplatin.

In another aspect, the invention features pharmaceutical compositions containing a covalent CDK7 inhibitor described herein, e.g., (a) an effective amount of Compound 1

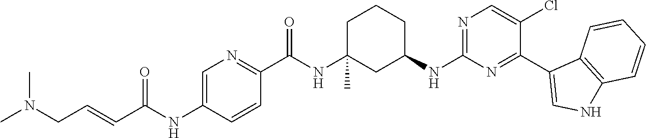 [SMILES: CN(C)C/C=C/C(=O)Nc1ccc(C(=O)N[C@@]2(C)CCC[C@@H](Nc3ncc(Cl)c(-c4c[nH]c5ccccc45)n3)C2)nc1]

or a pharmaceutically acceptable salt thereof; (b) an effective amount of a second agent selected from a Bcl-2 inhibitor such as venetoclax, a PARP inhibitor such as olaparib or niraparib, a platinum-based anti-cancer agent such as carboplatin or oxaliplatin, a taxane such as paclitaxel, a CDK4/6 inhibitor such as palbociclib, ribociclib, abemaciclib, or trilaciclib, a selective estrogen receptor modulator such as tamoxifen, and a selective estrogen receptor degrader such as fulvestrant; and (c) a pharmaceutically acceptable carrier.

In another aspect, the invention features methods of treating a human subject having a cancer, the method comprising administering to a subject identified as having a level of B-cell lymphoma-extra large (BCLXL) mRNA in the cancer equal to or below a pre-determined threshold an effective amount of N-((1S,3R)-3-(5-chloro-4-(1H-indol-3-yl)pyrimidin-2-ylamino)-1-methylcyclohexyl)-5-((E)-4-(dimethylamino)but-2-enamido)picolinamide (Compound 1).

In one embodiment, the invention features methods of treating cancer, the methods including a step of administering an effective amount of a covalent CDK7 inhibitor to a subject (e.g., a human subject) identified as having a level of B-cell lymphoma-extra large (BCLXL) mRNA in the cancer (e.g., in a biological sample obtained from the patient to be treated) that is equal to or below a pre-determined threshold (i.e., a “selected patient”). The methods can further include a step of determining the level of BCLXL mRNA present in a sample of cancer cells from the subject, and this is generally true for the methods of treatment described herein; regardless of the biomarker analyzed or the type of cancer in question, a method of treatment can either be carried out on an identified patient without an explicit step of analyzing the biomarker or with an explicit step in which the biomarker is analyzed (e.g., by obtaining a biological sample from a subject). The human subject may have been diagnosed as having a cancer sensitive to a covalent CDK7 inhibitor responsive to the determination, and the state of the BCLXL biomarker can be determined in any of the additional ways described herein. The pre-determined threshold is a cutoff value or a prevalence cutoff. A subject who is determined to have a cancer sensitive to a covalent CDK7 inhibitor can additionally be administered a Bcl-2 inhibitor (e.g., venetoclax (Venclexta®)), and a subject selected as described here (through an analysis of the state of BCLXL) can be suffering from a breast cancer, an ovarian cancer, a lung cancer, or a hematological cancer. More specifically, the subject can be suffering from TNBC, ovarian cancer, non-small cell lung cancer, or AML.

In one embodiment, the invention features methods of treating cancer, the methods including a step of administering an effective amount of a covalent CDK7 inhibitor to a subject (e.g., a human subject) identified as having a MYC SE, a MYC SE strength above a pre-determined threshold, a CDK18 SE, a CDK18 SE strength above a pre-determined threshold, an FGFR1 SE, or an FGFR1 SE strength above a pre-determined threshold. In some embodiments, the method further includes a step of analyzing the SE (e.g., by determining its presence or absence and/or its strength) in a biological sample including cancer cells from the subject. The human subject may have been diagnosed as having a cancer sensitive to a CDK7 inhibitor responsive to the determination. A subject selected as described here (through an analysis of the state of MYC, CDK8, or FGFR1) can be suffering from a breast cancer (e.g., TNBC). A diagnosing step can be based on the presence (or absence) or the strength of a MYC SE or a CDK18 SE. In one embodiment, the subject is suffering from ovarian cancer and the diagnosis is based on the presence or absence or the strength of an FGFR1 SE.

In one embodiment, the invention features methods of diagnosing and treating a human subject having a cancer, the method including the steps of: (a) diagnosing whether the subject has a cancer sensitive to a CDK7 inhibitor based on the state of a biomarker selected from CDK7, CDK9, CDK18 and CDK19 (e.g., a level of CDK7, CDK9, CDK18, or CDK19 mRNA) previously determined by analyzing a sample of cancer cells from the subject; and (b) administering an effective amount of a covalent CDK7 inhibitor to a subject identified as having a cancer, wherein either: (i) the state of the CDK18 or CDK19 biomarker (e.g., the CDK18 or CDK19 mRNA level) is equal to or above a pre-determined threshold, or (ii) the state of the CDK7 or CDK9 biomarker (e.g., the CDK7 or CDK9 mRNA level) is equal to or below a pre-determined threshold (i.e., the “selected subject”). These methods can further include determining the state of a CDK biomarker selected from CDK7, CDK9, CDK18 and CDK19 in the cancer cells of the subject; determining by an active analytical step that may include obtaining a biological sample from a subject. The subject may have been diagnosed as having a cancer sensitive to a CDK7 inhibitor responsive to the determination. As in other embodiments, the covalent CDK7 inhibitor can be a compound of Formula A (e.g., Compound 1). Where the biomarker is CDK7, CDK9, CDK18, or CDK19, the subject may have a lymphoma and the diagnosing step may more specifically be based on the level of CDK7 mRNA; the subject may have a TNBC, and the diagnosing step may more specifically be based on the level of CDK9 mRNA; the subject may have a TNBC, and the diagnosing step may more specifically be based on the level of CDK18 mRNA; the subject may have a TNBC or a small cell lung cancer, and the diagnosing step may more specifically be based on the level of CDK19 mRNA.

With regard to combination therapies and pharmaceutical compositions that include a first and a second active agent, the invention provides methods of treating a human subject having a cancer with a combination of a covalent CDK7 inhibitor (as described herein) and a CDK4/6 inhibitor. The covalent CDK7 inhibitor can be Compound 1. In any of these embodiments, the CDK4/6 inhibitor can be Ibrance® (palbociclib), Kisqali® (ribociclib), Verzenio® (abemaciclib), trilaciclib, G1T38, BPI-1178, or ON 123300, and the cancer to be treated can be a breast cancer, pancreatic cancer, lung cancer, or squamous cell cancer of the head and neck. More specifically, the cancer to be treated can be small cell lung cancer, non-small cell lung cancer, TNBC, an estrogen receptor-positive (ER⁺) breast cancer, pancreatic cancer or squamous cell cancer of the head and neck. Even more specifically, the CDK4/6 inhibitor can be Ibrance® (palbociclib), Kisqali® (ribociclib), Verzenio® (abemaciclib). Even more specifically, the cancer to be treated is an ER⁺ breast cancer.

The invention provides methods of treating a human subject having a cancer with a combination of a CDK7 inhibitor as described herein (e.g., a compound of Formula A (e.g., Compound 1)) and a CDK9 inhibitor (e.g., NVP2). The cancer to be treated can be a breast cancer and, more specifically, can be a Her2⁺/ER⁻/PR⁻ breast cancer.

The invention provides methods of treating a human subject having a cancer with a combination of a CDK7 inhibitor as described herein (e.g., a compound of Formula A (e.g., Compound 1)) and a Flt3 inhibitor (e.g., midostaurin). The cancer to be treated can be a hematological cancer (e.g., AML).

The invention provides methods of treating a human subject having a cancer with a combination of a CDK7 inhibitor as described herein (e.g., a compound of Formula A (e.g., Compound 1)) and a BET inhibitor. In this embodiment and others specifying a BET inhibitor, the BET inhibitor can be JQ1 or a compound disclosed in U.S. application Ser. No. 12/810,564, which is hereby incorporated herein by reference in its entirety. In this embodiment, the cancer to be treated can be a hematological cancer (e.g., AML) or a breast cancer (e.g., TNBC). In other embodiments of these methods, the cancer to be treated is Ewing's Sarcoma.

The invention provides methods of treating a human subject having a cancer with a combination of a covalent CDK7 inhibitor as described herein (e.g., a compound of Formula A or Compound 1) and a Bcl-2 inhibitor. As in other embodiments of the invention, the Bcl-2 inhibitor can be APG-1252, S55746, BP1002, APG-2575, or venetoclax. The cancer can be breast cancer (e.g., TNBC), an ovarian cancer, a lung cancer (e.g., NSCLC) or a hematological cancer (e.g., AML).

The invention provides methods of treating a human subject having a cancer with a combination of a covalent CDK7 inhibitor (e.g., a compound of Formula A or Compound 1) and a PARP inhibitor. As in other embodiments, the PARP inhibitor can be Zejula® (niraparib) or Lynpraza® (olaparib). In some embodiments of this method, the subject is suffering from a breast cancer (e.g., TNBC or Her2⁺/ER⁻/PR⁻ breast cancer), an ovarian cancer (e.g., an epithelial ovarian cancer), a fallopian tube cancer, or a primary peritoneal cancer.

The invention provides pharmaceutical kits for treating cancer comprising a covalent CDK7 inhibitor, which may be a compound of Formula A (e.g., Compound 1), and, optionally, a second therapeutic agent selected from: (a) a Bcl-2 inhibitor, (b) a CDK9 inhibitor, (c) a Flt3 inhibitor, (d) a PARP inhibitor, (e) a BET inhibitor, or (f) a CDK4/6 inhibitor, any of which may be selected from those disclosed herein. The kit can include optional instructions for: (a) reconstituting (if necessary) the covalent CDK7 inhibitor and/or the second therapeutic agent; (b) administering each of the covalent CDK7 inhibitor and/or the second therapeutic agent; and/or (c) a list of specific cancers for which the kit is useful or diagnostic methods by which they may be determined. The kit can also include any type of paraphernalia useful in administering the active agent(s) contained therein (e.g., tubing, syringes, needles, sterile dressings, tape, and the like).

The invention provides pharmaceutically acceptable combinations comprising a CDK7 inhibitor as described herein and a second therapeutic agent selected from: (a) a Bcl-2 inhibitor, (b) a CDK9 inhibitor, (c) a Flt3 inhibitor, (d) a PARP inhibitor, (e) a BET inhibitor, or (f) a CDK4/6 inhibitor (many examples of which are provided herein and can be incorporated); and a pharmaceutically acceptable carrier.

The invention provides methods of treating a human subject having a cancer, the method comprising: administering to a subject identified as having in at least one of the genes involved in the RB-E2F pathway: (1) an alteration in the DNA (e.g. gene copy number, mutation, methylation); (2) an epigenetic alteration (e.g. histone methylation, histone acetylation); or (3) an alteration in the level of expression of mRNA or protein, an effective amount of a covalent CDK7 inhibitor, as described herein. The subject is one identified (i.e., selected) as having an alteration in the level of mRNA expressed from at least one gene involved in the Rb-E2F pathway. In this aspect, the subject is determined to have either a level of mRNA of the at least one gene involved the RbE2F pathway equal to or above a pre-determined threshold or a level of mRNA of the at least one gene involved the RbE2F pathway equal to or below a pre-determined threshold, prior to administering to the subject an effective amount of a CDK7 inhibitor.

It will be readily apparent to one of ordinary skill in the art that for those genes in the RB-E2F pathway that are activated or overexpressed in cancer, one would select those patients that had (1) an alteration in the DNA encoding such gene that resulted in increased expression (e.g. elevated gene copy number, mutation that led to increased activity, change in methylation that led to increased expression); (2) an epigenetic alteration associated with that gene that resulted in increased expression (e.g. histone methylation or histone acetylation pattern that led to increased expression); or (3) an increase in the level of expression of mRNA or protein encoded by that gene. For those genes in the RB-E2F pathway that are inactivated or under-expressed in cancer, one would select from those patients that had (1) an alteration in the DNA encoding that gene that resulted in decreased expression or activity (e.g. reduced gene copy number, mutation that led to decreased activity or inactivity, change in methylation that led to decreased expression); (2) an epigenetic alteration associated with that gene that resulted in decreased expression (e.g. histone methylation or histone acetylation pattern that led to decreased expression); or (3) an decrease in the level of expression of mRNA or protein encoded by that gene.

In some aspects relating to using RB-E2F pathway genes as biomarkers, the invention provides a method of treating a human subject having a cancer, which comprises administering to a subject identified as having either (a) a level of CCNE1 mRNA or protein in the cancer equal to or above a pre-determined threshold; and/or (b) a level of RB1 mRNA or protein in the cancer equal to or below a pre-determined threshold, an effective amount of a CDK7 inhibitor. In some aspects of these embodiments, the method further comprises determining a level of RB1 and/or CCNE1 mRNA or protein present in a sample of cancer cells from the subject. In some aspects of these embodiments, the human subject is diagnosed as having a cancer sensitive to a CDK7 inhibitor responsive to the determination. In some aspects of these embodiments, the human subject is suffering from ovarian cancer. In some aspects of these embodiments, the human subject is suffering from a breast cancer. In some aspects of these embodiments, the human subject is suffering from a triple negative breast cancer (TNBC). In some aspects of these embodiments, the human subject is suffering from a hormone-receptor positive (HR⁺) breast cancer. In some aspect of these embodiments, the CDK7 inhibitor is Compound 1. In some aspects of these embodiments, the CDK7 inhibitor (e.g., Compound 1) is co-administered with a PARP inhibitor. In some embodiments the CDK7 inhibitor (e.g., Compound 1) is co-administered with a SERM or a SERD such as tamoxifen or fulvestrant.

The invention provides a method of treating a cancer in a human subject by administering to the subject a combination of a CDK7 inhibitor and a platinum-based standard of care anti-cancer agent for such cancer or a taxane. In some aspects of this embodiment, the cancer is an ovarian cancer. In some aspects of this thirteenth embodiment, the standard of care anti-cancer agent is a platinum-based anti-cancer agent. In some aspects of this embodiment, the CDK7 inhibitor is Compound 1. In some aspects of this embodiment, the platinum-based anti-cancer agent is carboplatin. In some aspects of this embodiment, the platinum-based anti-cancer agent is oxaliplatin. In some aspects of this embodiment, the human subject is, has been determined to be, or has become resistant (after some initial responsiveness) resistant to the platinum-based anti-cancer agent when administered as either a monotherapy or in combination with an anti-cancer agent other than a CDK7 inhibitor. In some aspects of this embodiment, the human subject is determined to have become resistant to the platinum-based anti-cancer agent when administered as a monotherapy or in combination with an anti-cancer agent other than a CDK7 inhibitor after some initial efficacy of that prior treatment. In some aspects of this embodiment, the standard of care anti-cancer agent is a taxane. In some aspects of this embodiment, the taxane is paclitaxel.

The invention provides a method of enhancing or prolonging the efficacy of a platinum-based anti-cancer agent in a human subject suffering from a cancer, by co-administering to the subject the platinum-based anti-cancer agent and a CDK 7 inhibitor. In some embodiments, the cancer is an ovarian cancer. In some embodiments, the CDK7 inhibitor is Compound 1. In some embodiments, the platinum-based anti-cancer agent is carboplatin or oxaliplatin.

The invention provides a method of treating HR⁺ breast cancer in a human subject selected on the basis of being resistant to treatment with a CDK4/6 inhibitor comprising the step of administering to the subject a covalent CDK 7 inhibitor (e.g., a compound of Formula A or Compound 1). In some embodiments, prior to administration of the CDK7 inhibitor (e.g., Compound 1), the subject is, has been determined to be, or has become resistant (after some initial responsiveness) to a prior treatment with a CDK4/6 inhibitor alone or in combination with another standard of care agent for breast cancer other than a CDK7 inhibitor, such as an aromatase inhibitor (e.g., letrozole, anastrozole) or a SERM or SERD such as tamoxifen or fulvestrant. In other words, the human subject is selected for treatment with a covalent CDK7 inhibitor (e.g., Compound 1) on the basis of being resistant to prior treatment with a CDK4/6 inhibitor alone or in combination with another standard of care agent for breast cancer other than a CDK7 inhibitor. In some embodiments, the covalent CDK7 inhibitor (e.g., Compound 1) is co-administered with another standard of care agent, such as an aromatase inhibitor (e.g. anastrozole, exemestane, or letrozole) or a SERM or SERD such as tamoxifen or fulvestrant, or a second line treatment after failure on an aromatase inhibitor or fulvestrant. In some embodiments, prior to administration of the covalent CDK7 inhibitor (e.g., Compound 1), the subject is, has been determined to be, or has become resistant (after some initial responsiveness) to treatment with a CDK4/6 inhibitor alone or in combination with another standard of care agent for breast cancer other than a CDK7 inhibitor, such as an aromatase inhibitor (e.g., anastrozole, exemestane, or letrozole), or a SERM or SERD such as tamoxifen or fulvestrant; and the covalent CDK7 inhibitor (e.g., Compound 1) is co-administered with a standard of care agent for breast cancer (e.g., a second line treatment after failure of an aromatase inhibitor or a SERM or SERD such as tamoxifen or fulvestrant.

The invention provides a method of diagnosing and treating a human subject having a cancer, the method comprising: (a) diagnosing whether the subject has a cancer sensitive to a CDK7 inhibitor based on the level of FGFR1, CDK6, CCND2, or CDKNA2, or the absence of a wild-type RB1 gene previously determined in a sample of cancer cells from the subject; and (b) administering an effective amount of a CDK7 inhibitor to a subject identified as having a cancer wherein: (a) the level of FGFR1, CDK6, or CCND2A mRNA is equal to or above a pre-determined threshold level; (b) the level of CDKN2A mRNA is equal to or below a pre-determined threshold level; or (c) the subject lacks the presence of a wild-type RB1 gene. In some aspects of these embodiments, the covalent CDK7 inhibitor is Compound 1. In some aspects of these embodiments, the cancer is ovarian cancer.

In a related embodiment, the invention provides methods of treating cancer in a human subject selected on the basis of the cancer having one or more of: (a) a level of FGFR1 mRNA equal to or above a pre-determined threshold level; (b) a level of CDK6 mRNA equal to or above a pre-determined threshold level; (c) a level of CCND2 mRNA equal to or above a pre-determined threshold level; (d) a level of CDKN2A mRNA equal to or below a pre-determined threshold level; or (e) an absence of a wild-type RB1 gene, wherein the selected subject is administered a covalent CDK7 inhibitor. In some embodiments, the covalent CDK7 inhibitor is Compound 1, and the cancer is ovarian cancer.

### BRIEF DESCRIPTION OF THE DRAWINGS

FIGS. 1A-1C show the results of microarrays comparing the level of MYC mRNA (FIG. 1A), MYC copy number (FIG. 1B), or the strength of a SE associated with MYC (FIG. 1C) in various human cancer cell lines to their sensitivity to Compound 1. The cell lines are partitioned by both cancer type and, for breast cancer, by cancer subtype, and for each cancer type or subtype, the cell's response to treatment with Compound 1 is indicated as high (gray bars) or low (black bars). SE strength was only tested in breast cancer cell lines. FIG. 1D shows the correlation between SE strength and MYC mRNA expression for TNBC cells that were responsive (gray dots) and non-responsive (black dots) to Compound 1.

FIG. 2 shows the results of a microarray comparing the level of CDK7 mRNA in various human cancer cell lines to their sensitivity to Compound 1. The cell lines are partitioned by cancer type and, for each cancer type, their response to treatment with Compound 1 (high or low).

FIG. 3 shows results of a microarray comparing the level of CDK9 mRNA in various human cancer cell lines to/with their sensitivity to Compound 1. The cell lines are partitioned by both cancer type and, for each cancer type, their response to treatment with Compound 1 (high or low).

FIG. 4 shows the results of a microarray comparing the level of CDK19 mRNA in various human cancer cell lines to/with their sensitivity to Compound 1. The cell lines are partitioned by both cancer type and, for each cancer type, their response to treatment with Compound 1 (high or low).

FIGS. 5A-5B show the results of assays comparing the presence of a super enhancer associated with CDK18 (FIG. 5A) or CDK18 mRNA measured in a microarray (FIG. 5B) in various human breast cancer cell lines to their sensitivity to Compound 1. The cell lines are further partitioned by TNBC- or non-TNBC breast cancer subtype and, for each cancer subtype, their response to treatment with Compound 1 (high or low).

FIGS. 6A-6B show the results of a microarray comparing the level of BCL-XL mRNA in various human cancer cell lines to their sensitivity to Compound 1 (FIG. 6A) or staurosporine (FIG. 6B). The cell lines are partitioned by both cancer type and, for each cancer type, their response to treatment with Compound 1 (high or low).

FIGS. 7A-7B show the results of a microarray comparing the level of CDK7 mRNA in various human cancer cell lines to their sensitivity to Compound 1 (FIG. 7A) or staurosporine (FIG. 7B). The cell lines are partitioned by both cancer type and, for each cancer type, their response to treatment with Compound 1 (high or low).

FIGS. 8A-8B show the results of a microarray comparing the level of CDK9 mRNA in various human cancer cell lines to their sensitivity to Compound 1 (FIG. 8A) or staurosporine (FIG. 8B). The cell lines are partitioned by both cancer type and, for each cancer type, their response to treatment with Compound 1 (high or low).

FIGS. 9A-9D show the results of treatment of a THP1 AML cell line with a combination of varying amounts of JQ1 and Compound 1 (FIGS. 9A and 9B). The results of combination treatment are plotted as a Combination Index (CI; FIG. 9C) and as an Isobologram (FIG. 9D).

FIGS. 10A-10D show the results of treating an AML3 AML cell line with a combination of varying amounts of JQ1 and Compound 1 (FIGS. 10A and 10B). The results of combination treatment are plotted as a Combination Index (CI; FIG. 10C) and as an Isobologram (FIG. 10D).

FIGS. 11A-11D show the results of treating an OCI-M1 AML cell line with a combination of varying amounts of JQ1 and Compound 1 (FIGS. 11A and 11B). The results of combination treatment are plotted as a Combination Index (CI; FIG. 11C) and as an Isobologram (FIG. 11D).

FIGS. 12A-12E show the results of treating an HL60 AML cell line with a combination of varying amounts of JQ1 and Compound 1 (FIGS. 12A and 12B). The results of combination treatment are plotted as a Combination Index (CI; FIG. 12C) and as an Isobologram (FIG. 12D). A comparison of cell line viability versus treatment is shown as a bar graph in FIG. 12E.

FIGS. 13A-13D show the results of treating a THP1 AML cell line with a combination of varying amounts of venetoclax and Compound 1 (FIGS. 13A and 13B). The results of combination treatment are plotted as a Combination Index (CI; FIG. 13C) and as an Isobologram (FIG. 13D).

FIGS. 14A-14C show the results of treating an AML3 AML cell line with a combination of varying amounts of venetoclax and Compound 1 (FIGS. 14A and 14B). The results of combination treatment are plotted as an Isobologram (FIG. 14C).

FIGS. 15A-15E show the results of treating a HL60 AML cell line with a combination of varying amounts of venetoclax and Compound 1 (FIGS. 15A and 15B). The results of combination treatment are plotted as a Combination Index (CI; FIG. 15C), as an Isobologram (FIG. 15D). A comparison of cell line viability versus treatment is shown as a bar graph in FIG. 15E.

FIGS. 16A-16D show the results of treating a THP1 AML cell line with a combination of varying amounts of the Flt3 inhibitor midostaurin and Compound 1 (FIGS. 16A and 16B). The results of combination treatment are plotted as a Combination Index (CI; FIG. 16C) and as an Isobologram (FIG. 16D).

FIGS. 17A-17D show the results of treating an AML3 AML cell line with a combination of varying amounts of the Flt3 inhibitor midostaurin and Compound 1 (FIGS. 17A and 17B). The results of combination treatment are plotted as a Combination Index (CI; FIG. 17C) and as an Isobologram (FIG. 17D).

FIGS. 18A-18D show the results of treating a MV411 AML cell line with a combination of varying amounts of the Flt3 inhibitor midostaurin and Compound 1 (FIGS. 18A and 18B). The results of combination treatment are plotted as a Combination Index (CI; FIG. 18C) and as an Isobologram (FIG. 18D).

FIGS. 19A-19E show the results of treating a AU565 breast cancer cell line with a combination of varying amounts of the CDK9 inhibitor NVP2 and Compound 1 (FIGS. 19A and 19B). The results of combination treatment are plotted as a Combination Index (CI; FIG. 19C), as an Isobologram (FIG. 19D). A comparison of cell line viability versus treatment is shown as a bar graph in FIG. 19E.

FIGS. 20A-20E show the results of treating a HCC38 TNBC breast cancer cell line with a combination of varying amounts of the PARP inhibitor niraparib and Compound 1 (FIGS. 20A and 20B). The results of combination treatment are plotted as a Combination Index (CI; FIG. 20C), as an Isobologram (FIG. 20D). A comparison of cell line viability versus treatment is shown as a bar graph in FIG. 20E.

FIGS. 21A-21E show the results of treating a AU565 breast cancer cell line with a combination of varying amounts of the PARP inhibitor niraparib and Compound 1 (FIGS. 21A and 21B). The results of combination treatment are plotted as a Combination Index (CI; FIG. 21C), as an Isobologram (FIG. 21D). A comparison of cell line viability versus treatment is shown as a bar graph in FIG. 21E.

FIGS. 22A-22B show the results of a microarray comparing the level of BCL2L1 (which encodes BCL-XL) mRNA in various human cancer cell lines (FIG. 22A) and in subsets of breast cancer cell lines (FIG. 22B) to their sensitivity to Compound 1. The cell lines in these figures are partitioned by both cancer type or breast cancer subtype and, for each cancer type or subtype, their response to treatment with Compound 1 (high or low).

FIGS. 23A-23D show the effect of Compound 1 on the expression of various BCL2 family members in breast cancer cell lines and ovarian cancer cells at the protein level (FIGS. 23A and 23D) and mRNA level (FIGS. 23B and 23C).

FIG. 24A shows the level of BCL2 protein in four different AML cell lines. FIG. 24B shows the effect of Compound 1 on the level of BCL-XL and MCL1 proteins in those same four AML cell lines.

FIG. 25 is an isobologram showing the combined effect of Compound 1 and venetoclax on the AML cell line KG1.

FIGS. 26A-26C show the results of treatment of a T47D breast cancer cell line with a combination of varying amounts of the CDK4/6 inhibitor palbociclib and Compound 1 (FIG. 26A). The results of combination treatment are plotted as a Combination Index (CI; FIG. 26B), as an Isobologram (FIG. 26C).

FIGS. 27A-27C show the results of treatment of a T47D breast cancer cell line with a combination of varying amounts of the CDK4/6 inhibitor ribociclib and Compound 1 (FIG. 27A). The results of combination treatment are plotted as a Combination Index (CI; FIG. 27B), as an Isobologram (FIG. 27C).

FIGS. 28A-28C show the results of treatment of a T47D breast cancer cell line with a combination of varying amounts of the CDK4/6 inhibitor abemaciclib and Compound 1 (FIG. 28A). The results of combination treatment are plotted as a Combination Index (CI; FIG. 28B), as an Isobologram (FIG. 28C).

FIGS. 29A-29B show Western blots of the protein expression levels of various biomarkers (MCL1, BCLXL, and BCL2 in FIG. 29A; MCL1 in FIG. 29B) as compared to β-actin expression levels after treatment with different amounts of Compound 1 in TNBC cell lines (FIG. 29A) and in a HCC70 tumor xenograft model (FIG. 29B).

FIGS. 30A-30B show that the correlation between growth rate (GR) of various TNBC cell lines and varying amounts of Compound 1 is dependent upon BCLXL protein expression level. Growth rate of four different cell lines is shown in FIG. 30A. Baseline BCLXL protein expression in those four cell lines is shown in FIG. 30B.

FIG. 31 shows the effect of Compound 1 on tumor volume in a human TNBC cell line (HCC70) xenograft.

FIGS. 32A-32D show the effect of Compound 1 on tumor volume in four different human TNBC patient sample xenografts. Each black line in FIGS. 32A-32D represents a different xenograft mouse. Gray lines represent historical tumor growth in individual untreated mice.

FIGS. 33A-33C show the mRNA expression of biomarkers (FIG. 33A—BCL2L1; FIG. 33B—CCNE1), and the CCNE1 gene copy number (FIG. 33C) in TNBC patient sample xenografts.

FIGS. 34A-34H show the effect of Compound 1 on tumor volume in nine different human ovarian cancer patient sample xenografts. Each gray line represents a different xenograft mouse. Black lines represent historical tumor growth in individual untreated mice.

FIG. 35 shows CCNE1 and RB1 protein levels in the eight ovarian cancer xenografts analyzed in FIGS. 34A-34H.

FIG. 36 is an isobologram showing the combined effect of Compound 1 and venetoclax on the AML cell line ML-2.

FIG. 37 is an isobologram showing the combined effect of Compound 1 and venetoclax on the AML cell line KG-1.

FIG. 38 shows the effect of no treatment, venetoclax alone (50 mg/kg, once a day “QD”), Compound 1 alone (40 mg/kg, once a week “QW”), or a combination of venetoclax (50 mg/kg QD) and Compound 1 (40 mg/kg QW) on the tumor size in a KG-1 xenograft.

FIG. 39A are isobolograms showing the combined effect of Compound 1 and carboplatin for different ovarian cancer cell lines. FIG. 39B is a dose-response growth curve for A2780 cells treated with varying amounts of Compound 1 and/or carboplatin.

FIGS. 40A-40B are isobolograms showing the combined effect of Compound 1 and oxaliplatin for different ovarian cancer cell lines.

FIGS. 41A-41B are isobolograms showing the combined effect of Compound 1 and the PARP inhibitor olaparib for different ovarian cancer cell lines.

FIGS. 42A-42B are isobolograms showing the combined effect of Compound 1 and the taxane paclitaxel for different ovarian cancer cell lines.

FIG. 43 depicts the effect of Compound 1 versus vehicle control on the RNA expression of CHEK1, CHEK2 and RAD51 genes in THP-1 AML cells.

FIG. 44 depicts the effect of Compound 1, or taxol versus vehicle and untreated controls on the RNA expression of CHEK1, CHEK2 and RAD51 genes in various breast cancer lines.

FIGS. 45A-45E depict the rank of all enhancers in ovarian cancer patient xenograft model OV15612, showing a super-enhancer associated with FGFR1 (FIG. 45A), as well mRNA levels (FGFR1—FIG. 45B; CDK6—FIG. 45C; CCND2—FIG. 45D) and FGFR1 protein levels in OV15612 (FIG. 45E) and other ovarian cancer patient xenograft models.

FIG. 46 depicts CDKN2A mRNA expression in various ovarian cancer patient xenograft models.

FIG. 47 depicts the effect of a combination of various amounts of Compound 1 with various amounts JQ1 on the growth of the Ewing's Sarcoma cell line SKES.

FIGS. 48A-48B depicts the effect of a combination of various amounts of Compound 1 with various amounts JQ1 on the growth of the Ewing's Sarcoma cell line RDES (FIG. 48A) and an isobologram showing the combined effect of Compound 1 and JQ1 on that cell line (FIG. 48B).

FIGS. 49A-49B depicts the effect of a combination of various amounts of Compound 1 with various amounts JQ1 on the growth of the Ewing's Sarcoma cell line A674 (FIG. 49A) and an isobologram showing the combined effect of Compound 1 and JQ1 on that cell line (FIG. 49B).

FIGS. 50A-50B depicts the effect of a combination of various amounts of Compound 1 with various amounts JQ1 on the growth of the osteocarcinoma cell line Saos2 (FIG. 50) and an isobologram showing the combined effect of Compound 1 and JQ1 on that cell line (FIG. 50).

FIG. 51 depicts the level of expression of genes related to homologous recombination deficiency and carboplatin sensitivity in ovarian cell line A2780 after treatment with Compound 1 for 0, 6 and 16 hours.

FIG. 52 depicts the level of expression of genes related to homologous recombination deficiency and carboplatin sensitivity in ovarian cell line COV318 after treatment with Compound 1 for 0, 6 and 16 hours.

FIG. 53 depicts the level of expression of genes related to homologous recombination deficiency and carboplatin sensitivity in ovarian cell line TOV21G after treatment with Compound 1 for 0, 6 and 16 hours.

FIG. 54 depicts the level of expression of genes related to homologous recombination deficiency and carboplatin sensitivity in ovarian cell line OvCar3 after treatment with Compound 1 for 0, 6 and 16 hours.

FIG. 55 depicts the effects of carboplatin alone, Compound 1 alone, and a combination of carboplatin and Compound 1 on tumor volume in a TOV21G xenograft model

FIG. 56 depicts the effects of carboplatin alone, Compound 1 alone, and a combination of carboplatin and Compound 1 on tumor volume in an OvCar3 xenograft model

FIG. 57 depicts the effects of carboplatin alone, Compound 1 alone, and a combination of carboplatin and Compound 1 on tumor volume in a A2780 xenograft model

### DETAILED DESCRIPTION

N-((1S,3R)-3-(5-chloro-4-(1H-indol-3-yl)pyrimidin-2-ylamino)-1-methylcyclohexyl)-5-((E)-4-(dimethylamino)but-2-enamido)picolinamide (Compound 1):

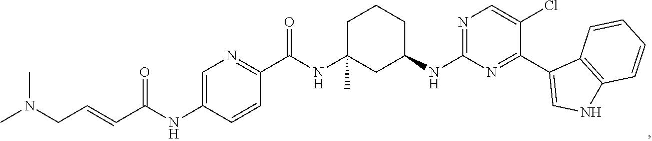 [SMILES: CN(C)C/C=C/C(=O)Nc1ccc(C(=O)N[C@@]2(C)CCC[C@@H](Nc3ncc(Cl)c(-c4c[nH]c5ccccc45)n3)C2)nc1]

a covalent and selective inhibitor of CDK7, was developed to exploit dysregulated programs thought to drive SE-mediated transcriptional-dependencies in cancers (see WO 2015/154039). Compound 1 has previously been shown to selectively induce apoptosis in leukemic cells relative to non-malignant cells in vitro, and it has demonstrated anti-tumor activity in AML xenografts.

Despite the efficacy of Compound 1, we believe that such efficacy will be higher in subjects that have certain genetic signatures (i.e., biomarkers in a particular state, as described herein). Moreover, we also believe that the efficacy of Compound 1 may be enhanced when combined with other classes of anti-oncogenic/anti-cancer therapies or vice versa.

As used herein, the term “biological sample” refers to any sample obtained from an individual (e.g., a patient or subject or an animal model) suffering from a disease (or, in the case of an animal model, a simulation of that disease) to be diagnosed or treated by the methods of this invention or from an individual serving in the capacity of a reference or control (or whose sample contributes to a reference standard or control population). The biological sample can be a tissue sample, such as a tissue section or tissue obtained by biopsy (e.g., by needle biopsy or surgical biopsy); a cell sample obtained from, for example, the Papanicolaou test or blood smears; a cell sample obtained by, for example, microdissection; a bone marrow sample (e.g., a sample of either whole bone marrow, complete cell fractions thereof, or subpopulations of cells therein); tissue of a xenograft, or a cell fraction consisting of cellular fragments, cellular organelles, and/or nucleic acids that may be obtained by lysing cells and separating the components thereof by centrifugation or otherwise. Other examples of biological samples include saliva, blood, serum, urine, semen, fecal matter, cerebrospinal fluid (CSF), interstitial fluid, mucus, tears, sweat, vaginal fluid, swabs (such as buccal swabs). A biological sample can be obtained from a solid tumor (e.g., a tumor of the breast, ovary, lung, or any other cancer-affected organ disclosed herein) or a hematological tumor. For example, the biological sample from a subject suffering from a hematological cancer (e.g., a leukemia (e.g., AML)) can be a bone marrow aspirate, fractionated whole blood, a PBMC (peripheral blood mononuclear cell) fraction from the subject's whole blood, or a PBMC sample further enriched for specific blasts using various enrichment techniques such as antibody-linked bead enrichment protocols, fluorescent label cell sorting, or other techniques known in the art. In some embodiments, as will be clear from the context in which it is described or used, the term “biological sample” refers to a preparation that is obtained by processing a primary sample (e.g., by removing one or more components of and/or by adding one or more agents to the primary sample). Such a “processed sample” may comprise, for example, nucleic acids or proteins extracted from a sample or obtained by subjecting a primary sample to techniques such as amplification or reverse transcription of mRNA, isolation and/or purification of certain components, etc.

With regard to certain values, as will be clear from the context, the terms “about” and “approximately” are used to describe standard variation as would be understood by one of ordinary skill in the art or a range within plus-or-minus 10% (e.g., plus-or minus 1%) of the stated value. For example, a prevalence rank in a population of about 80% means a prevalence rank of 72-88% (e.g., 79.2-80.8%). In case of doubt, “about X” can be “X” (e.g., about 80% can be 80%).

As used herein, the term “biomarker” refers to an entity whose state correlates with a particular biological event so that it is considered to be a “marker” for that event (e.g., the presence of a particular cancer and its susceptibility to a covalent CDK7 inhibitor). A biomarker can be analyzed at the nucleic acid or protein level; at the nucleic acid level, one can analyze the presence, absence, or copy number of a gene in wild type or mutant form, its association with a super-enhancer, and/or its level of expression (as evidenced, for example, by mRNA levels). At the protein level, one can analyze the level of expression and/or activity of a protein encoded by a genetic biomarker gene. In some embodiments, a biomarker may indicate a therapeutic outcome or likelihood thereof. Thus, a biomarker can be predictive, prognostic, or diagnostic and is therefore useful in methods of identifying (thereby diagnosing) or treating a patient as described herein.

As used herein, the term “cancer” refers to a disease in which cells exhibit relatively abnormal, uncontrolled, and/or autonomous growth, resulting in an aberrant growth phenotype characterized by loss of control of cell proliferation to an extent detrimental to the patient having the disease. Intrinsic factors (e.g., a genetic mutation) and/or extrinsic factors (e.g., exposure to a pathogen or carcinogen) may have contributed to a patient's cancer. Further, the cancer can be classified by the type of tissue in which it originated (histological type) and/or by the primary site in the body in which the cancer first developed. Based on histological type, cancers are generally grouped into six major categories: carcinomas; sarcomas; myelomas; leukemias; lymphomas; and mixed types. A cancer treated as described herein may be of any one of these types and may comprise cells that are precancerous (e.g., benign), malignant, pre-metastatic, metastatic, and/or non-metastatic. A patient who has a malignancy or malignant lesion has a cancer. A relevant cancer may be characterized by a solid tumor or by a hematologic tumor, which may also be known as a blood cancer. In any of the embodiments of the invention in which a patient is suffering from a blood cancer, it can be a leukemia such as acute lymphocytic leukemia (ALL; e.g., B cell ALL or T cell ALL), acute myelocytic leukemia (AML; e.g., B cell AML or T cell AML), chronic myelocytic leukemia (CML; e.g., B cell CML or T cell CML), or chronic lymphocytic leukemia (CLL; e.g., B cell CLL (e.g., harry cell leukemia) or T cell CLL). The blood cancer can also be a lymphoma such as Hodgkin lymphoma (HL; e.g., B cell HL or T cell HL), non-Hodgkin lymphoma (NHL; e.g., B cell NHL or T cell NHL), follicular lymphoma, chronic lymphocytic leukemia/small lymphocytic lymphoma (CLL/SLL), mantle cell lymphoma (MCL), a marginal zone B cell lymphoma (e.g., splenic marginal zone B cell lymphoma), primary mediastinal B cell lymphoma, Burkitt lymphoma, lymphoplasmacytic lymphoma (i.e., Waldenstrom's macroglobulinemia), immunoblastic large cell lymphoma, precursor B-lymphoblastic lymphoma, or primary central nervous system (CNS) lymphoma. The B cell NHL can be diffuse large cell lymphoma (DLCL; e.g., diffuse large B cell lymphoma), and the T cell NHL can be precursor T lymphoblastic lymphoma or a peripheral T cell lymphoma (PTCL). In turn, the PTCL can be a cutaneous T cell lymphoma (CTCL) such as mycosis fungoides or Sezary syndrome, angioimmunoblastic T cell lymphoma, extranodal natural kill T cell lymphoma, enteropathy type T cell lymphoma, subcutaneous anniculitis-like T cell lymphoma, or anaplastic large cell lymphoma. We may use the term “cancer” to refer to a tumor or malignant neoplasm (Stedman's Medical Dictionary, 25th ed.; Hensly ed.; Williams & Wilkins: Philadelphia, 1990). As indicated, a cancer can manifest as an abnormal mass of tissue whose growth surpasses and is not coordinated with the growth of a normal tissue. A malignant neoplasm is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

As used herein, the term “combination therapy” is used to refer to those situations in which a subject is exposed to two or more therapeutic regimens (e.g., two or more therapeutic agents). The two or more therapeutic regimens may be administered simultaneously, sequentially, or in overlapping dosing regimens. “Administration” of a combination therapy may involve administration of one or more agents to a subject who is receiving the other agent(s) in the combination. For clarity, while combination therapy does not require that individual agents be administered together in a single composition (or even necessarily at the same time), administration of a single composition containing both agents and co-incident administration are within the meaning of “combination therapy.” As noted above, the invention features pharmaceutical kits for treating a cancer patient, and those kits can include a covalent CDK7 inhibitor and instructions to administer it to a cancer patient who has undergone, is undergoing, or will undergo treatment with a second, specified therapeutic agent (i.e., a patient who was or is considered a candidate for treatment with the second agent). When a patient receives the first and second agents sequentially (i.e., when a patient has undergone or will undergo treatment with an anti-cancer agent other than the covalent CDK7 inhibitor), the length of time between the administrations can vary significantly (e.g., it can be a matter of hours, days, weeks, or months) but will be such that one of ordinary skill in the art would view the sequential administrations as constituting a combination therapy for the cancer for which the patient is currently being treated.

As used herein, the term “covalent,” as it relates to an inhibitor of CDK7, refers to the manner in which the inhibitor interacts with CDK7 at a molecular level; a covalent inhibitor of CDK7 forms a chemical bond with CDK7 in which at least one pair of electrons is shared between an atom in the inhibitor and an atom in CDK7. The result is inhibition of an activity of CDK7 in a way that benefits a patient who has cancer. We use the terms “covalent inhibitor of CDK7” and “covalent CDK7 inhibitor” interchangeably.

As used herein, the terms “cutoff” and “cutoff value” mean a value measured in an assay that defines the dividing line between two subsets of a population (e.g., responders and non-responders (e.g., responders and non-responders to a CDK7 inhibitor). Thus, values that are equal to or higher than the cutoff value defines one subset of the population, and values that are lower than the cutoff value defines the other subset of the population.

As used herein, “diagnostic information” is information that is useful in determining whether a patient has a disease and/or in classifying (stratifying) the disease into a genotypic or phenotypic category or any category having significance with regard to the prognosis of the disease or its likely response to treatment (either treatment in general or any particular treatment described herein). Similarly, “diagnosis” refers to obtaining or providing any type of diagnostic information, including, but not limited to, whether a subject is likely to have or develop a disease; whether that disease has or is likely to reach a certain state or stage or to exhibit a particular characteristic (e.g., resistance to a therapeutic agent); information related to the nature or classification of a tumor; information related to prognosis (which may also concern resistance); and/or information useful in selecting an appropriate treatment (e.g., selecting a covalent CDK7 inhibitor for a patient identified as having a cancer that is likely to respond to such an inhibitor or other treatment). A patient classified (stratified) according to a method described herein and selected for treatment with a covalent CDK7 inhibitor is likely to respond well to the treatment, meaning that such a patient is more likely to be successfully treated than a patient with the same type of cancer who has not been so identified and is not in the same strata. Available treatments include therapeutic agents and other treatment modalities such as surgery, radiation, etc., and selecting an appropriate treatment encompasses the choice of withholding a particular therapeutic agent; the choice of a dosing regimen; and the choice of employing a combination therapy. Diagnostic information can be used to stratify patients and is thus useful in identifying and classifying a given patient according to, for example, biomarker status. Obtaining diagnostic information can constitute a step in any of the patient stratification methods described herein.

One of ordinary skill in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

One of ordinary skill in the art will appreciate that the term “dosing regimen” may be used to refer to a set of unit doses (typically more than one) that are administered individually to a subject, separated by equal or unequal periods of time. A given therapeutic agent typically has a recommended dosing regimen, which may involve one or more doses, each of which may contain the same unit dose amount or differing amounts. In some embodiments, a dosing regimen comprises a first dose in a first dose amount, followed by one or more additional doses in a second dose amount that is different from the first dose amount. In some embodiments, a dosing regimen is correlated with a desired or beneficial outcome when administered across a relevant population (i.e., the regimen is a therapeutic dosing regimen).

As used herein, an “effective amount” of an agent (e.g., a chemical compound described herein), such as a compound of Formula (A) or Compound 1, refers to an amount that produces or is expected to produce the desired effect for which it is administered. The effective amount will vary depending on factors such as the desired biological endpoint, the pharmacokinetics of the compound administered, the condition being treated, the mode of administration, and characteristics of the subject, as discussed further below and recognized in the art. The term can be applied to therapeutic and prophylactic methods. For example, a therapeutically effective amount is one that reduces the incidence and/or severity of one or more signs or symptoms of the disease. For example, in treating a cancer, an effective amount may reduce the tumor burden, stop tumor growth, inhibit metastasis or prolong patient survival. One of ordinary skill in the art will appreciate that the term does not in fact require successful treatment be achieved in any particular individual. Rather, a therapeutically effective amount is that amount that provides a particular desired pharmacological response in a significant number of subjects when administered to patients in need of such treatment. In some embodiments, reference to a therapeutically effective amount may be a reference to an amount administered or an amount measured in one or more specific tissues (e.g., a tissue affected by the disease) or fluids (e.g., blood, saliva, serum, sweat, tears, urine, etc.). Effective amounts may be formulated and/or administered in a single dose or in a plurality of doses, for example, as part of a dosing regimen.

As used herein, an “enhancer” is a region of genomic DNA that helps regulate the expression of genes up to 1 Mbp away. An enhancer may overlap, but is often not composed of, gene coding regions. An enhancer is often bound by transcription factors and designated by specific histone marks.

As used herein, the term “patient” or “subject” are used interchangeably and each refers to any organism to which a provided composition is or may be administered, e.g., for experimental, diagnostic, prophylactic, and/or therapeutic purposes. Typical patients include animals (e.g., mammals such as mice, rats, rabbits, dogs, cats, non-human primates, and humans). The patient (e.g., a human) may be treated pre-natally or post-natally, and genetically-born males or females may be subjected to a method described herein regardless of the stage of life at which diagnosis and/or treatment may be advised (e.g., a patient can be an infant, child, adolescent, young adult, middle-aged adult, or senior adult). A patient may be suffering from or susceptible to one or more diseases, disorders, or conditions and may display one or more signs or symptoms of a disease, disorder, or condition. We may tend to use the term “subject” when referring to an individual subjected to a diagnostic method or who has provided a biological sample for reference or for analysis within a reference population. Similarly, we may tend to use the term “patient” when referring to an individual subjected to a therapeutic method. However, it is to be understood that any individual, whether human or not, and whether designated as a “patient” or a “subject” can be subjected to the diagnostic methods described herein, the therapeutic methods described herein, or both.

As used herein, the term “pharmaceutically acceptable salt” refers to a salt that is, within the scope of sound medical judgment, suitable for use in contact with the tissues of humans and lower animals without undue toxicity, irritation, allergic response and the like, and is commensurate with a reasonable benefit/risk ratio. Pharmaceutically acceptable salts are well known in the art (see Berge et al., J. Pharmaceutical Sciences, 66:1-19, 1977, incorporated herein by reference). Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, MALAT1e, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, the term “population” means some number of items (e.g., at least 30, 40, 50, or more) sufficient to reasonably reflect the distribution, in a larger group, of the value being measured in the population. Within the context of the present invention, the population can be a discrete group of humans, laboratory animals, or cells lines (for example) that are identified by at least one common characteristic for the purposes of data collection and analysis. For example, a “population of samples” refers to a plurality of samples that is large enough to reasonably reflect the distribution of a value (e.g., a value related to the state of a biomarker) in a larger group of samples. The items in the population may be biological samples, as described herein. For example, each sample in a population of samples may be cells of a cell line or a biological sample obtained from a subject or a xenograft (e.g., a tumor grown in a mouse by implanting a tumorigenic cell line or a patient sample into the mouse). As noted, individuals within a population can be a discrete group identified by a common characteristic, which can be the same disease, condition, or disorder (e.g., the same type of cancer), whether the sample is obtained from living beings suffering from the same type of cancer or a cell line or xenograft representing that cancer.

The term “prevalence cutoff,” as used herein in reference to a specified value (e.g., the strength of a SE associated a biomarker disclosed herein) means the prevalence rank that defines the dividing line between two subsets of a population (e.g., a subset of “responders” and a subset of “non-responders,” which, as the names imply include subjects who are likely or unlikely, respectively, to experience a beneficial response to a therapeutic agent or agents). Thus, a prevalence rank that is equal to or higher (e.g., a lower percentage value) than the prevalence cutoff defines one subset of the population; and a prevalence rank that is lower (e.g., a higher percentage value) than the prevalence cutoff defines the other subset of the population.

As used herein, the term “prevalence rank” for a specified value (e.g., the mRNA level of a specific biomarker) means the percentage of a population that are equal to or greater than that specific value. For example, a 35% prevalence rank for the amount of mRNA of a specific biomarker in a test cell means that 35% of the population have that level of biomarker mRNA or greater than the test cell.

As used herein, the terms “prognostic information” and “predictive information” are used to refer to any information that may be used to indicate any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

As used herein, the term “rank ordering” means the ordering of values from highest to lowest or from lowest to highest.

As used herein, the terms “Rb-E2F pathway” and “Rb-E2F family” refer to a set of genes whose expression regulates the activity of the RB gene family, which in turn regulates the activity of the E2F family of transcription factors that are required for entry into and progression through the cell cycle. The following table contains a list of genes in the RB-E2F family, an indication of the functions of the encoded proteins, and the status of these biomarkers in cancer. We use the shorthand “activated or overexpressed” to indicate that the copy number, or level of expression of this gene is known to be higher in certain cancers as compared to healthy subjects. In some aspects of this invention, the pre-determined threshold for such activated or overexpressed genes is the level (e.g., mRNA level, protein level, gene copy number, strength of enhancer associated with the gene) that is present in a cancer patient known to have a higher level than a healthy subject. We use the shorthand “inactivated or underexpressed” to indicate that the copy number, or level of expression of this gene is known to be lower in patients having certain cancers as compared to healthy subjects. In some aspects of this invention, the pre-determined threshold for such inactivated or underexpressed genes is the level (e.g., mRNA level, protein level, gene copy number, strength of enhancer associated with the gene) that is present in a cancer patient known to have a lower level than a healthy subject.

| Gene | Function | Status in Cancer |
|---|---|---|
| E2F1 | E2F family - transcriptional | Activated or overexpressed |
| | control of cell cycle entry | |
| E2F2 | E2F family - transcriptional | Activated or overexpressed |
| | control of cell cycle entry | |
| E2F3 | E2F family - transcriptional | Activated or overexpressed |
| | control of cell cycle entry | |
| E2F4 | E2F family - transcriptional | Activated or overexpressed |
| | control of cell cycle entry | |
| E2F5 | E2F family - transcriptional | Activated or overexpressed |
| | control of cell cycle entry | |
| E2F6 | E2F family - transcriptional | Activated or overexpressed |
| | control of cell cycle entry | |
| E2F7 | E2F family - transcriptional | Activated or overexpressed |
| | control of cell cycle entry | |
| E2F8 | E2F family - transcriptional | Activated or overexpressed |
| | control of cell cycle entry | |
| RB1 | RB family - E2F family | Inactivated or underexpressed |
| | inhibition | |
| RBL1 | RB family - E2F family | Inactivated or underexpressed |
| | inhibition | |
| RBL2 | RB family - E2F family | Inactivated or underexpressed |
| | inhibition | |
| CDK4 | RB family inhibition | Activated or overexpressed |
| CDK6 | RB family inhibition | Activated or overexpressed |
| CDK2 | RB family inhibition | Activated or overexpressed |
| CCND1 | CDK4/6 regulation | Activated or overexpressed |
| CCND2 | CDK4/6 regulation | Activated or overexpressed |
| CCND3 | CDK4/6 regulation | Activated or overexpressed |
| CDKN2A | CDK4/6 regulation | Inactivated or underexpressed |
| CDKN2B | CDK4/6 regulation | Inactivated or underexpressed |
| CDKN2C | CDK4/6 regulation | Inactivated or underexpressed |
| CDKN2D | CDK4/6 regulation | Inactivated or underexpressed |
| CCNE1 | CDK2 regulation | Activated or overexpressed |
| CCNE2 | CDK2 regulation | Activated or overexpressed |
| CDKN1A | CDK2 regulation | Inactivated or underexpressed |
| CDKN1B | CDK2 regulation | Inactivated or underexpressed |
| CDKN1C | CDK2 regulation | Inactivated or underexpressed |
| FBXW7 | CCNE regulation | Inactivated or underexpressed |

As used herein, a “reference” refers to a standard or control relative to which a comparison is performed. For example, an agent, subject (or patient), population, sample, sequence, or value of interest is compared with a reference agent, subject (or patient), population, sample, sequence or value. The reference can be analyzed or determined substantially simultaneously with the analysis or determination of the item of interest or it may constitute a historical standard or control, determined at an earlier point in time and optionally embodied in a tangible medium. One of ordinary skill in the art is well trained in selecting appropriate references, which are typically determined or characterized under conditions that are comparable to those encountered by the item of interest. One of ordinary skill in the art will appreciate when sufficient similarities are present to justify reliance on and/or comparison to a particular possible reference as a standard or control.

As used herein, a “response” to treatment is any beneficial alteration in a subject's condition that results from, or that correlates with, treatment. The alteration may be stabilization of the condition (e.g., inhibition of deterioration that would have taken place in the absence of the treatment), amelioration of, delay of onset of, and/or reduction in frequency of one or more signs or symptoms of the condition, improvement in the prospects for cure of the condition, greater survival time, and etc. A response may be a subject's response or a tumor's response.

As used herein, when the term “strength” is used to refer to a portion of an enhancer or a SE, it means the area under the curve of the number of H3K27Ac or other genomic marker reads plotted against the length of the genomic DNA segment analyzed. Thus, “strength” is an integration of the signal resulting from measuring the mark at a given base pair over the span of the base pairs defining the region being chosen to measure.

As used herein, the term “super-enhancer” (SE) refers to a subset of enhancers that contain a disproportionate share of histone marks and/or transcriptional proteins relative to other enhancers in a particular cell or cell type. Genes regulated by SEs are predicted to be of high importance to the function of a cell. SEs are typically determined by rank ordering all of the enhancers in a cell based on strength and determining, using available software such as ROSE (bitbucket.org/young computation/rose), the subset of enhancers that have significantly higher strength than the median enhancer in the cell (see, e.g., U.S. Pat. No. 9,181,580, which is hereby incorporated by reference herein in its entirety).

As used herein, the terms “threshold” and “threshold level” mean a level that defines the dividing line between two subsets of a population (e.g., responders and non-responders). A threshold or threshold level may be a prevalence cutoff or a cutoff value.

As used herein, the terms “treatment,” “treat,” and “treating” (and other grammatical variants thereof) refer to reversing, alleviating, delaying the onset of, and/or inhibiting the progress of a “pathological condition” (e.g., a disease, disorder, or condition, or one or more signs or symptoms thereof) described herein. In some embodiments, “treatment,” “treat,” and “treating” require that signs or symptoms of the disease disorder or condition have developed or have been observed. In other embodiments, treatment may be administered in the absence of signs or symptoms of the disease or condition (e.g., in light of a history of symptoms and/or in light of genetic or other susceptibility factors). Treatment may also be continued after symptoms have resolved, for example, to delay or inhibit recurrence.

The terms “condition,” “disease,” and “disorder” are used interchangeably herein unless the context clearly indicates otherwise. Similarly, as the invention relates to compositions and methods for diagnosing and treating patients who have cancer, the terms “active agent,” “anti-cancer agent,” “pharmaceutical agent,” and “therapeutic agent” are used interchangeably (unless the context clearly indicates otherwise) and the compounds described herein as covalent inhibitors of CDK7, including Compound 1 and those conforming to Formula (A), would be understood by one of ordinary skill in the art as active, anti-cancer, pharmaceutical, or therapeutic agents.

The compositions and methods described herein, including pharmaceutical kits and compositions, therapies employing one or more active agents, and diagnostic or patient stratification methods, can include or employ any covalent inhibitor of CDK7, particularly covalent inhibitors of CDK7 that form a covalent bond with the —SH group of Cys312 of CDK7 or an equivalent cysteine residue in a mutant form of CDK7 (i.e., the same cysteine residue but bearing a different amino acid number because of amino acid insertions and/or deletions in such mutants). Such covalent inhibitors of CDK7 will contain an electrophilic moiety that is capable of reacting with the nucleophilic —SH moiety of CDK7 Cys312 to form a covalent bond between the inhibitor and Cys312.

Covalent CDK7 inhibitors suitable for use in the compositions and methods described herein include those conforming to structural formula (A):

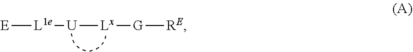

and pharmaceutically acceptable salts thereof.

E is a chemical group that is moderately hydrophobic with a fragment cLogP between 1.0 and 3.0, which associates with a hydrophobic pocket exposed by the inactive form of CDK7 characterized by a closed conformation of the activation loop (DFG “out”), and may optionally contain at least one hydrogen bond donor moiety that forms a hydrogen bond to the nitrogen of CDK7 residue lysine 41 (Lys41);

L^{1e} is a linker group ranging from 0 to 3 atoms in length;

R^{E} is an electrophilic group that forms a covalent bond with the —SH group of Cys312 of CDK7;

U is a chemical group that contains at least one hydrogen bond acceptor moiety that forms a hydrogen bond to the backbone amide —NH— of CDK7 residue methionine 94 (Met94);

L^{X} is a linker group ranging from 0 to 5 atoms in length, which may contain at least one hydrogen bond donor moiety that forms a hydrogen bond to the backbone amide —CO— group of CDK7 residue methionine 94 (Met94);

U and L^{X} may be optionally taken together to form a cyclic structure; and

G is a chemical group that spans a total length of approximately 20 to approximately 30 \acute{Å}.

In some embodiments, E, of Formula A, is an optionally substituted heteroaryl group. More specifically, E can be an optionally substituted heteroaryl ring of any one of the Formulae (i-1)-(i-6):

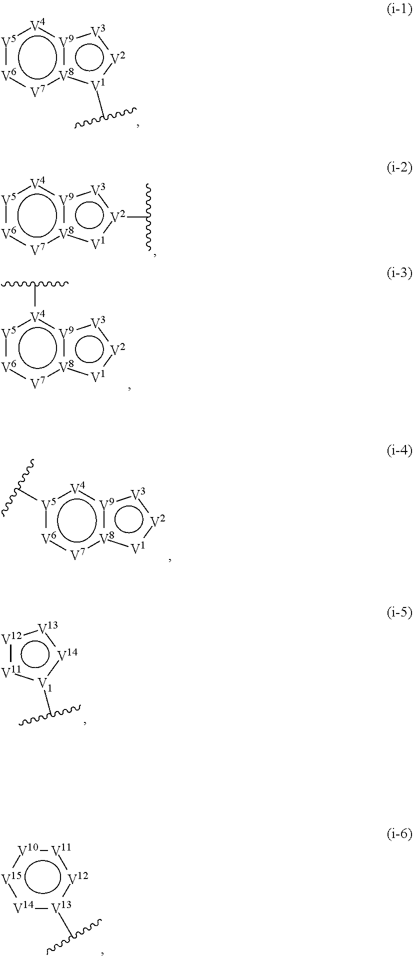

wherein:

each instance of V¹, V², V³, V⁴, V⁵, V⁶, V⁷, V⁸, V⁹, V¹⁰, V¹¹, V¹², V¹³, V¹⁴ and V¹⁵ is independently O, S, N, N(R^{A1}), C, or C(R^{A2});

each instance of R^{A1} is independently selected from hydrogen, deuterium, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl;

each instance of R^{A2} is independently selected from hydrogen, deuterium, halogen, —CN, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A2a}, —N(R^{A2a})₂, and —SR^{A2a}, wherein each occurrence of R^{A2a} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl, or

any two R^{A1}, any two R^{A2}, or one R^{A1} and one R^{A2} are joined to form an optionally substituted carbocyclic, optionally substituted heterocyclic, optionally substituted aryl, or optionally substituted heteroaryl ring.

In some embodiments, U and L^{X} are not taken together to form a heteroaryl; and U is a nitrogen-containing heteroaryl. U can be an optionally substituted pyrimidine.

In some embodiments, G consists of two cyclic moieties bound to one another through a 1 to 3 atom linker. In some embodiments, the cyclic moiety in G bound to R^{E} is an optionally substituted aryl or heteroaryl ring. In more specific embodiments, the cyclic moiety in G bound to R^{E} is an optionally substituted phenyl or pyridinyl ring. The cyclic moiety in G bound to L^{X} can be an optionally cycloalkyl or saturated heterocyclyl ring. The cyclic moiety in G bound to L^{X} can be an optionally substituted cyclohexyl ring.

In some embodiments, R^{E} is any one of the Formulae (ii-1)-(ii-19):

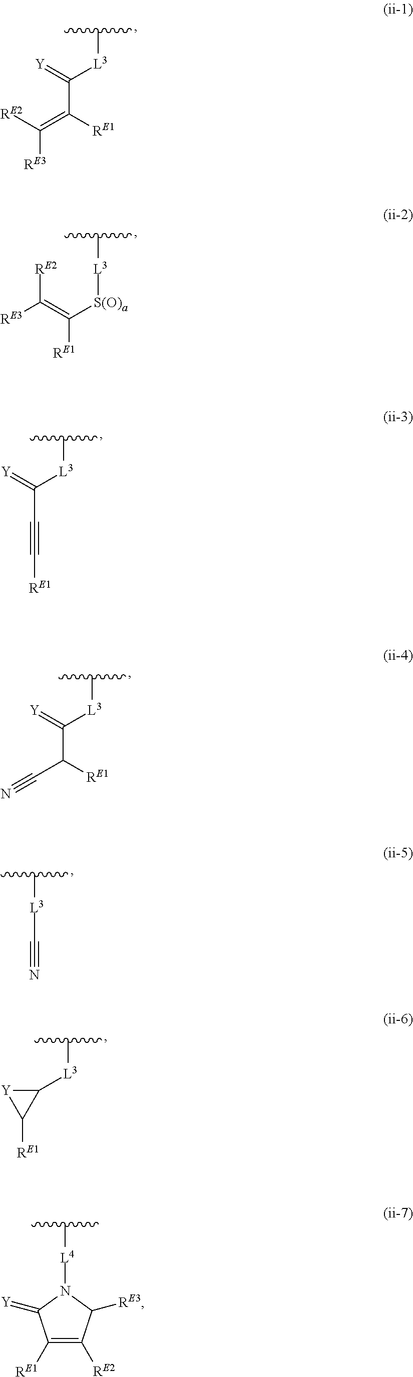

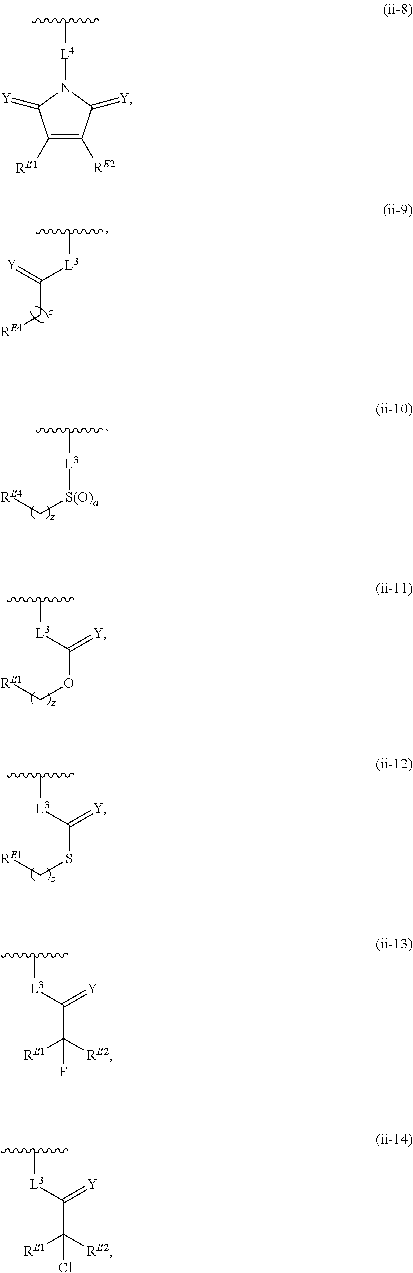

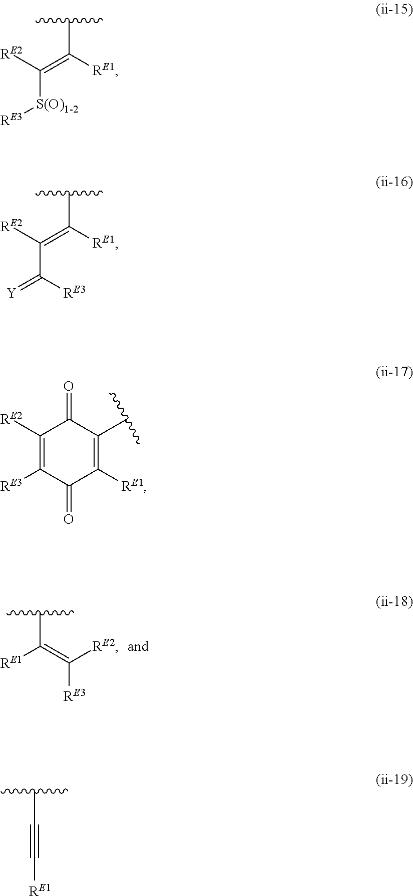

wherein:

L³ is a bond, an optionally substituted C₁-C₇ alkylene, or an optionally substituted C₂-C₇ alkenylene or alkynylene, wherein one or more methylene units of the alkylene, alkenylene or alkynylene are optionally and independently replaced with —O—, —S—, —S(O)—, —S(O)₂, or —N(R⁶)—;

L⁴ is a bond, an optionally substituted C₁-C₄ alkylene, or an optionally substituted C₂-C₄ alkenylene or alkynylene;

each of R^{E1}, R^{E2} and R^{E3} is independently selected from hydrogen, deuterium, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CH₂OR⁹, —CH₂N(R⁹)₂, —CH₂SR⁹, —CN, —OR⁹, —N(R⁹)₂, and —SR⁹, wherein each occurrence of R⁹ is independently selected from hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R⁹ are taken together to form an optionally substituted heterocyclyl, or

R^{E1} and R^{E3}, or R^{E2} and R^{E3}, or R^{E1} and R^{E2} are joined to form an optionally substituted carbocyclic or optionally substituted heterocyclic ring;

R^{E4} is a leaving group;

Y is O, S, or N(R⁶), wherein R⁶ is hydrogen, or —C₁-C₆ alkyl; and

z is 0, 1, 2, 3, 4, 5, or 6.

Examples of the above-described covalent CDK7 inhibitors can be found in PCT publications WO2014063068 (U.S. application Ser. No. 14/436,496), WO2015058126 (U.S. application Ser. No. 15/030,249), WO2015058163 (U.S. application Ser. No. 15/030,265), WO2015058140 (U.S. application Ser. No. 15/030,245), WO2015154039 (U.S. application Ser. No. 15/301,815), and WO2015154022 (U.S. application Ser. No. 15/301,819), the disclosures of which are hereby incorporated herein by reference in their entireties.

In some embodiments, the covalent CDK7 inhibitor is Compound 1 or a pharmaceutically acceptable salt thereof; in some embodiments, the covalent CDK7 inhibitor is Compound 1.

An enhancer or SE can be identified by various methods known in the art (see Hinsz et al., Cell, 155:934-947, 2013; McKeown et al., Cancer Discov., 7(10):1136-53, 2017; and PCT/US2013/066957, each of which are hereby incorporated herein by reference in their entireties). Identifying a SE can be achieved by obtaining a biological sample from a patient (e.g., from a biopsy or other source, as described herein). The important metrics for enhancer measurement occur in two dimensions: along the length of the DNA over which genomic markers (e.g., H3K27Ac) are contiguously detected and the compiled incidence of genomic marker at each base pair along that span of DNA, the compiled incidence constituting the magnitude. The measurement of the area under the curve (“AUC”) resulting from integration of length and magnitude analyses determines the strength of the enhancer. The strength of the MYC, CDK18, CDK19, CCNE1, or FGFR1 SEs relative to an appropriate reference can be used to diagnose (stratify) a patient and thereby determine whether a subject is likely to respond well to Compound 1 or another of the covalent CDK7 inhibitors described herein. It will be readily apparent to one of ordinary skill in the art, particularly in view of the instant specification, that if the length of DNA over which the genomic markers is detected is the same for each of MYC, CDK18, CDK19, CCNE1, or FGFR1 and the reference/control, then the ratio of the magnitude of the MYC, CDK18, CDK19, CCNE1, or FGFR1 SE relative to the control will be equivalent to the strength and may also be used to determine whether a subject will be responsive to a covalent CDK7 inhibitor (such as Compound 1 or another compound described herein). The strength of the MYC, CDK18, CDK19, CCNE1, or FGFR1 SE in a cell can be normalized before comparing it to other samples. Normalization is achieved by comparison to a region in the same cell known to comprise a ubiquitous SE or enhancer that is present at similar levels in all cells. One example of such a ubiquitous super-enhancer region is the MALAT1 super-enhancer locus (chr11:65263724-65266724) (genome build hg19).

It has been determined through H3K27Ac ChIP-seq (ChIP-sequencing) methods that there is a SE locus associated with the MYC gene at chr8:128628088-128778308; a SE locus associated with the CDK18 gene at chr1:205399084-205515396; a SE locus associated with the CDK19 gene at chr6:110803523-110896277; a SE locus associated with the CCNE1 gene at chr19:30418503-30441450; and a SE locus associated with the FGFR1 gene at chr8:38233326-38595483. All loci are based on the Gencode v19 annotation of the human genome build hg19/GRCh37.

ChIP-seq is used to analyze protein interactions with DNA by combining chromatin immunoprecipitation (ChIP) with massively parallel DNA sequencing to identify the binding sites of DNA-associated proteins. It can be used to map global binding sites precisely for any protein of interest. Previously, ChIP-on-chip was the most common technique utilized to study these protein—DNA relations. Successful ChIP-seq is dependent on many factors including sonication strength and method, buffer compositions, antibody quality, and cell number (see, e.g., Furey, Nature Reviews Genetics 13:840-852, 2012); Metzker, Nature Reviews Genetics 11:31-46, 2010; and Park, Nature Reviews Genetics 10:669-680, 2009). Genomic markers other than H3K27Ac that can be used to identify SEs using ChIP-seq include P300, CBP, BRD2, BRD3, BRD4, components of the mediator complex (Loven et al., Cell, 153(2):320-334, 2013), histone 3 lysine 4 monomethylated (H3K4me1), and other tissue-specific enhancer tied transcription factors (Smith and Shilatifard, Nature Struct. Mol. Biol., 21(3):210-219, 2014; and Pott and Lieb, Nature Genetics, 47(1):8-12, 2015). Quantification of enhancer strength and identification of SEs can be determined using SE scores (McKeown et al., Cancer Discov. 7(10):1136-1153, 2017; DOI: 10.1158/2159-8290.CD-17-0399).

In some instances, H3K27Ac or other marker ChIP-seq data SE maps of the entire genome of a cell line or a patient sample already exist. One would then simply determine whether the strength or ordinal rank of the enhancer or SE in such maps at the chr8:128628088-128778308 (genome build hg19) locus was equal to or above the pre-determined threshold level. In some embodiments, one would simply determine whether the strength, or ordinal rank of the enhancer or super-enhancer in such maps at the chr1:205399084-205515396 (genome build hg19) locus was equal to or above the pre-determined threshold level.

It should be understood that the specific chromosomal location of MYC, CDK18, CDK19, CCNE1, or FGFR1 and MALAT1 may differ for different genome builds and/or for different cell types. However, one of ordinary skill in the art, particularly in view of the instant specification, can determine such different locations by locating in such other genome builds specific sequences corresponding to the MYC, CDK18, CDK19, CCNE1, or FGFR1 and/or MALAT1 loci in genome build hg 19.

Other methods that can be used to identify SEs in the context of the present methods include chromatin immunoprecipitation (Delmore et al., Cell, 146(6)904-917, 2011), chip array (ChIP-chip), and chromatin immunoprecipitation followed by qPCR (ChIP-qPCR) using the same immunoprecipitated genomic markers and oligonucleotide sequences that hybridize to the chr8:128628088-128778308 (genome build hg19) MYC locus or chr1:205399084-205515396 (genome build hg19) CDK18 locus (for example). In the case of ChIP-chip, the signal is typically detected by intensity fluorescence resulting from hybridization of a probe and input assay sample as with other array-based technologies. For ChIP-qPCR, a dye that becomes fluorescent after intercalating the double stranded DNA generated in the PCR reaction is used to measure amplification of the template.

In some embodiments, determination of whether a cell has a MYC, CDK18, CDK19, CCNE1, or FGFR1 SE strength equal to or above a requisite threshold level is achieved by comparing MYC, CDK18, CDK19, CCNE1, or FGFR1 enhancer strength in a test cell to the corresponding MYC, CDK18, CDK19, CCNE1, or FGFR1 strength in a population of cell samples, wherein each of the cell samples is obtained from a different source (e.g., a different subject, a different cell line, a different xenograft) reflecting the same disease to be treated. In some embodiments, only primary tumor cell samples from subjects are used to determine the threshold level. In some aspects of these embodiments, at least some of the samples in the population will have been tested for responsiveness to a specific CDK7 inhibitor (e.g., Compound 1) to establish: (a) the lowest MYC, CDK18, CDK19, CCNE1, or FGFR1 enhancer strength of a sample in the population that responds to that specific compound (“lowest responder”); and, optionally, (b) the highest MYC, CDK18, CDK19, CCNE1, or FGFR1 enhancer strength of a sample in the population that does not respond to that specific compound (“highest non-responder”). In these embodiments, a cutoff of MYC, CDK18, CDK19, CCNE1, or FGFR1 enhancer strength above which a test cell would be considered responsive to that specific compound is set: i) equal to or up to 5% above the MYC, CDK18, CDK19, CCNE1, or FGFR1 enhancer strength in the lowest responder in the population; or ii) equal to or up to 5% above the MYC, CDK18, CDK19, CCNE1, or FGFR1 enhancer strength in the highest non-responder in the population; or iii) a value in between the MYC, CDK18, CDK19, CCNE1, or FGFR1 enhancer strength of the lowest responder and the highest non-responder in the population.

In the above embodiments, not all of the samples in a population necessarily are to be tested for responsiveness to a specific CDK7 inhibitor (e.g., Compound 1), but all samples are measured for MYC, CDK18, CDK19, CCNE1, or FGFR1 enhancer strength. In some embodiments, the samples are rank ordered based on MYC, CDK18, CDK19, CCNE1, or FGFR1 enhancer strength. The choice of which of the three methods set forth above to use to establish the cutoff will depend upon the difference in MYC, CDK18, CDK19, CCNE1, or FGFR1 enhancer strength between the lowest responder and the highest non-responder in the population and whether the goal is to minimize the number of false positives or to minimize the chance of missing a potentially responsive sample or subject. When the difference between the lowest responder and highest non-responder is large (e.g., when there are many samples not tested for responsiveness that fall between the lowest responder and the highest non-responder in a rank ordering of MYC, CDK18, CDK19, CCNE1, or FGFR1 enhancer strength), the cutoff is typically set equal to or is up to 5% above the MYC, CDK18, CDK19, CCNE1, or FGFR1 enhancer strength in the lowest responder in the population. This cutoff maximizes the number of potential responders. When this difference is small (e.g., when there are few or no samples untested for responsiveness that fall between the lowest responder and the highest non-responder in a rank ordering of MYC, CDK18, CDK19, CCNE1, or FGFR1 enhancer strength), the cutoff is typically set to a value in between the MYC, CDK18, CDK19, CCNE1, or FGFR1 enhancer strength of the lowest responder and the highest non-responder. This cutoff minimizes the number of false positives. When the highest non-responder has a MYC, CDK18, CDK19, CCNE1, or FGFR1 enhancer strength that is greater than the lowest responder, the cutoff is typically set to a value equal to or up to 5% above the MYC, CDK18, CDK19, CCNE1, or FGFR1 enhancer strength in the highest non-responder in the population. This method also minimizes the number of false positives.

In some embodiments, the methods discussed above can be employed to simply determine if a diseased cell (e.g., a cancer cell) from a subject has a SE associated with a biomarker as described herein (e.g., MYC, CDK18, CDK19, CCNE1, or FGFR1 or a protein encoded thereby). The presence of the SE indicates that the subject is likely to respond well to a covalent CDK7 inhibitor (e.g., Compound 1). The cell is determined to have a SE associated with the biomarker (e.g., MYC, CDK18, CDK19, CCNE1, or FGFR1 or a protein encoded thereby) when the enhancer has a strength that is equal to or above the enhancer associated with MALAT-1. In alternate embodiments, the cell is determined to have a SE associated with MYC, CDK18, CDK19, CCNE1, or FGFR1 when the MYC, CDK18, CDK19, CCNE1, or FGFR1-associated enhancer has a strength that is at least 10-fold greater than the median strength of all of the enhancers in the cell. In other embodiments, the cell is determined to have a SE associated with MYC, CDK18, CDK19, CCNE1, or FGFR1 when the MYC, CDK18, CDK19, CCNE1, or FGFR1-associated enhancer has a strength that is above the point where the slope of the tangent is 1 in a rank-ordered graph of strength of each of the enhancers in the cell.

In embodiments involving CDK18, the cutoff value for enhancer strength can be converted to a prevalence cutoff, which can then be applied to CDK18 mRNA levels to determine a mRNA cutoff value in a given mRNA assay.

In some embodiments, mRNA levels of various genes of interest according to this invention (e.g., BCL-XL, CDK7, CDK9, CDK18, CDK19, CCNE1 or RB1) are used to determine sensitivity to a covalent CDK7 inhibitor (e.g., Compound 1).

In some embodiments, gene of interest/biomarker mRNA levels in a subject (as assessed, e.g., in a biological sample obtained from the subject) are compared, using the same assay, to the same gene of interest/biomarker mRNA levels in a population of subjects having the same disease or condition to identify likely responders to a covalent CDK7 inhibitor (a compound of Formula A or Compound 1). In embodiments where a biomarker is one whose mRNA expression correlates with responsiveness to Compound 1 (e.g., CDK18, CDK19, and CCNE1), at least some of the samples in the population will have been tested for responsiveness to the inhibitor (e.g., Compound 1) to establish: (a) the lowest mRNA level of a sample in the population that responds to that specific Compound 1 (“lowest mRNA responder”); and, optionally, (b) the highest mRNA level of a sample in the population that does not respond to that specific Compound 1 (“highest mRNA non-responder”). In these embodiments, a cutoff of biomarker mRNA level above which a test cell would be considered responsive to that specific Compound 1 is set: i) equal to or up to 5% above the mRNA level in the lowest mRNA responder in the population; or ii) equal to or up to 5% above the mRNA level in the highest mRNA non-responder in the population; or iii) a value in between the mRNA level of the lowest mRNA responder and the highest mRNA non-responder in the population.

In embodiments where mRNA levels positively correlate with sensitivity to Compound 1, not all of the samples in a population need to be tested for responsiveness to a Compound 1, but all samples are measured for the gene of interest mRNA levels. In some embodiments, the samples are rank ordered based on gene of interest mRNA levels. The choice of which of the three methods set forth above to use to establish the cutoff will depend upon the difference in gene of interest mRNA levels between the lowest mRNA responder and the highest mRNA non-responder in the population and whether the cutoff is designed to minimize false positives or maximize the potential number of responders. When this difference is large (e.g., when there are many samples not tested for responsiveness that fall between the lowest mRNA responder and the highest mRNA non-responder in a rank ordering of mRNA levels), the cutoff is typically set equal to or up to 5% above the mRNA level in the lowest mRNA responder. When this difference is small (e.g., when there are few or no samples untested for responsiveness that fall between the lowest mRNA responder and the highest mRNA non-responder in a rank ordering of mRNA levels), the cutoff is typically set to a value in between the mRNA levels of the lowest mRNA responder and the highest mRNA non-responder. When the highest mRNA non-responder has a mRNA level that is greater than the lowest mRNA responder, the cutoff is typically set to a value equal to or up to 5% above the mRNA levels in the highest mRNA non-responder in the population.

In embodiments where a gene of interest/biomarker is one whose mRNA expression inversely correlates with responsiveness to Compound 1 (i.e., BCL-XL, CDK7, CDK9, and RB1), at least some of the samples in the population will have been tested for responsiveness to Compound 1 in order to establish: (a) the highest mRNA level of a sample in the population that responds to that specific Compound 1 (“highest mRNA responder”); and, optionally, (b) the lowest mRNA level of a sample in the population that does not respond to that specific Compound 1 (“lowest mRNA non-responder”). In these embodiments, a cutoff of mRNA level above which a test cell would be considered responsive to that specific Compound 1 is set: i) equal to or up to 5% below the mRNA level in the highest mRNA responder in the population; or ii) equal to or up to 5% below the mRNA level in the lowest mRNA non-responder in the population; or iii) a value in between the mRNA level of the lowest mRNA non-responder and the highest mRNA responder and in the population.

In embodiments where mRNA levels inversely correlate with sensitivity to Compound 1, not all of the samples in a population need to be tested for responsiveness to a Compound 1, but all samples are measured for the gene of interest mRNA levels. In some embodiments, the samples are rank ordered based on gene of interest mRNA levels. The choice of which of the three methods set forth above to use to establish the cutoff will depend upon the difference in gene of interest mRNA levels between the highest mRNA responder and the lowest mRNA non-responder in the population and whether the cutoff is designed to minimize false positives or maximize the potential number of responders. When this difference is large (e.g., when there are many samples not tested for responsiveness that fall between the highest mRNA responder and the lowest mRNA non-responder in a rank ordering of mRNA levels), the cutoff is typically set equal to or up to 5% below the mRNA level in the highest mRNA responder. When this difference is small (e.g., when there are few or no samples untested for responsiveness that fall between the highest mRNA responder and the lowest mRNA non-responder in a rank ordering of mRNA levels), the cutoff is typically set to a value in between the mRNA levels of the highest mRNA responder and the lowest mRNA non-responder. When the highest mRNA responder has a mRNA level that is lower than the lowest mRNA responder, the cutoff is typically set to a value equal to or up to 5% below the mRNA levels in the lowest mRNA non-responder in the population.

In embodiments involving CDK18, the cutoff for CDK18 mRNA levels may be determined using the prevalence cutoff established based on CDK18 enhancer strength, as described above. In some aspects of these embodiments, a population is measured for mRNA levels and the prior determined prevalence cutoff is applied to that population to determine an mRNA cutoff level. In some aspects of these embodiments a rank-order standard curve of CDK18 mRNA levels in a population is created, and the pre-determined prevalence cutoff is applied to that standard curve to determine the CDK18 mRNA cutoff level.

In some aspects of embodiments where a test cell or sample is compared to a population, the cutoff mRNA level value(s) obtained for the population is converted to a prevalence rank and the mRNA level cutoff is expressed as a percent of the population having the cutoff value or higher, e.g., a prevalence cutoff.

Without being bound by theory, applicants believe that the prevalence rank of a test sample and the prevalence cutoff in a population will be similar regardless of the methodology used to determine mRNA levels.

A subject can be identified as likely to respond well to a covalent CDK7 inhibitor (e.g., a compound of Formula A or Compound 1) if the state of MYC, CDK18, CDK19, CCNE1, or FGFR1 (as determined by, e.g., mRNA levels in a biological sample from the subject) corresponds to (e.g., is equal to or greater than) a prevalence rank in a population of about 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 43%, 42%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, or 20% as determined by the state of MYC, CDK18, CDK19, CCNE1, or FGFR1, respectively, determined by assessing the same parameter (e.g., mRNA level(s)) in the population. A subject can be identified as likely to respond well to a covalent CDK7 inhibitor (e.g., a compound of Formula A or Compound 1) if the state of BCL-XL, CDK7 or CDK9 (as determined by, e.g., mRNA levels in a biological sample from the subject) is below a prevalence rank in a population of about 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 43%, 42%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, or 20% as determined by the state of BCL-XL, CDK7 or CDK9, respectively, determined by assessing the same parameter (e.g., mRNA level(s)) in the population. In some embodiments, the cutoff value or threshold is established based on the biomarker (e.g., mRNA) prevalence value.

In still other embodiments, a population may be divided into three groups: responders, partial responders and non-responders, and two cutoff values (or thresholds) or prevalence cutoffs are set or determined. The partial responder group may include responders and non-responders as well as those subjects whose response to a covalent CDK7 inhibitor (e.g., Compound 1) was not as high as the responder group. This type of stratification may be particularly useful when, in a population, the highest mRNA non-responder has an mRNA level that is greater than that of the lowest mRNA responder. In this scenario, for CDK18 or CDK19, the cutoff level or prevalence cutoff between responders and partial responders is set equal to or up to 5% above the CDK18 or CDK19 mRNA level of the highest CDK18 or CDK19 mRNA non-responder; and the cutoff level or prevalence cutoff between partial responders and non-responders is set equal to or up to 5% below the CDK18 or CDK19 mRNA level of the lowest CDK18 or CDK19 mRNA responder. For BCL-XL, CDK7 or CDK9, this type of stratification may be useful when the highest mRNA responder has a mRNA level that is lower than that of the lowest mRNA non-responder. In this scenario, for BCL-XL, CDK7 or CDK9, the cutoff level or prevalence cutoff between responders and partial responders is set equal to or up to 5% below the mRNA level of the lowest mRNA non-responder; and the cutoff level or prevalence cutoff between partial responders and non-responders is set equal to or up to 5% above the mRNA level of the highest mRNA responder. The determination of whether partial responders should be administered a covalent CDK7 inhibitor (e.g., Compound 1) will depend upon the judgment of the treating physician and/or approval by a regulatory agency.

Methods that can be used to quantify specific RNA sequences in a biological sample are known in the art and include, but are not limited to, fluorescent hybridization such as utilized in services and products provided by NanoString Technologies, array based technology (Affymetrix), reverse transcriptase qPCR as with SYBR® Green (Life Technologies) or TaqMan® technology (Life Technologies), RNA sequencing (e.g., RNA-seq), RNA hybridization and signal amplification as utilized with RNAscope® (Advanced Cell Diagnostics), or Northern blot. In some cases, mRNA expression values for various genes in various cell types are publicly available (see, e.g., broadinstitute.org/ccle; and Barretina et al., Nature, 483:603-607, 2012).

In some embodiments, the state of a biomarker (as assessed, for example, by the level of RNA transcripts) in both the test biological sample and the reference standard or all members of a population is normalized before comparison. Normalization involves adjusting the determined level of an RNA transcript by comparison to either another RNA transcript that is native to and present at equivalent levels in both of the cells (e.g., GADPH mRNA, 18S RNA), or to a fixed level of exogenous RNA that is “spiked” into samples of each of the cells prior to super-enhancer strength determination (Lovén et al., Cell, 151(3):476-82, 2012; Kanno et al., BMC Genomics 7:64, 2006; Van de Peppel et al., EMBO Rep., 4:387-93, 2003).

A subject (e.g., a human) suffering from a cancer described herein may have been determined to be resistant (or to be acquiring resistance after some initial efficacy) to a therapeutic agent that was administered prior to the covalent CDK7 inhibitor (e.g., Compound 1). That prior therapeutic agent may be a platinum-based anti-cancer agent administered as a monotherapy or in combination with a standard of care.

Most cancer subjects eventually develop resistance to platinum-based therapies by one or more of the following mechanisms: (i) molecular alterations in cell membrane transport proteins that decrease uptake of the platinum agent; (ii) molecular alterations in apoptotic signaling pathways that prevent a cell from inducing cell death; (iii) molecular alterations of certain genes (e.g. BRCA1/2, CHEK1, CHEK2, RAD51) that restore the ability of the cell to repair platinum agent-induced DNA damage. K. N. Yamamoto et al., 2014, PloS ONE 9(8):e105724. The term “molecular alterations” includes increased or decreased mRNA expression from the genes involved in these functions; increased or decreased expression of protein from such genes; and mutations in the mRNA/proteins expressed from those genes.

Resistance is typically determined by disease progression (e.g., an increase in tumor size and/or numbers) during treatment or a decrease in the rate of shrinkage of a tumor. In some instances, a patient will be considered to have become resistant to a platinum-based agent when the patient's cancer responds or stabilizes while on treatment, but which progresses within 1-6 months following treatment with the agent. Resistance can occur after any number of treatments with platinum agents. In some instances, disease progression occurs during, or within 1 month of completing treatment. In this case, the patient is considered to have never demonstrated a response to the agent. This is also referred to a being “refractory” to the treatment. Resistance may also be determined by a treating physician when the platinum agent is no longer considered to be an effective treatment for the cancer.

In some embodiments, the subject is, has been determined to be, or has become resistant to treatment with a CDK4/6 inhibitor administered as a monotherapy or in combination with a standard of care.

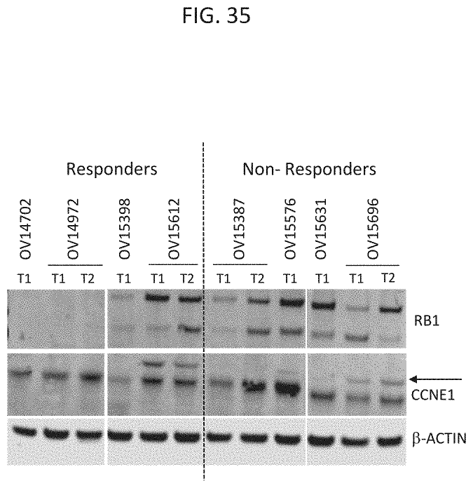

CDK4/6 inhibitors in cancer (e.g., HR⁺ breast cancer) are known to block entry into S phase of the cell cycle by inducing G1 arrest. Resistance to CDK4/6 inhibitors in cancer (e.g., HR⁺ metastatic breast cancer) has been shown to be mediated, in part, by molecular alterations that: 1) enhance CDK4/6 activity, such as amplifications of CDK6, CCND1, or FGFR1 (Formisano et al., SABCS 2017, Publication Number GS6-05; Cruz et al., SABCS 2017 Publication Number PD4-05), or 2) reactivate cell cycle entry downstream of CDK4/6, such as RB1 loss and CCNE1 amplification (Condorelli, Ann Oncol, 2017 PMID: 29236940; Herrera-Abreu MT, Cancer Research 2016 PMID: 27020857). As shown in FIG. 35, responses to Compound 1 in ovarian PDX models was associated with low RB1 expression or high CCNE1 expression. Further genetic and molecular analyses of these ovarian PDXs has revealed additional features in support of the antitumor activity of Compound 1 in tumors with molecular alterations that otherwise confer CDK4/6 inhibitor resistance (see Example 6).

In addition, Compound 1 inhibits many HR⁺ breast cancer cells in vitro, including those with a form of acquired resistance to aromatase inhibitors, as well as those that no longer respond to inhibitors of CDK4/6 (data not shown). Fulvestrant is a second line standard of care for breast cancer patients who have failed aromatase inhibitor treatment. In addition, it is known that the CDK7 inhibitor THZ-1 showed in vitro synergy with fulvestrant in several HR⁺ breast cancer cell lines (Jeselsohn et al., Cancer Cell, 33:173-86, 2018). This provides a mechanistic rationale for the efficacy of Compound 1 in combination with fulvestrant in patients with HR⁺ metastatic breast cancer who have progressed following treatment with a CDK4/6 inhibitor plus an aromatase inhibitor.

Unlike platinum-based agents which are typically administered for a period of time followed by a period without treatment, CDK4/6 inhibitors, such as palbociclib, ribociclib or abemaciclib, are administered until disease progression is observed. In some instances, a patient will be considered to have become resistant to a CDK4/6 inhibitor when the patient's cancer initially responds or stabilizes while on treatment, but which ultimately begins to progress while still on treatment. In some instances, a patient will be considered to be resistant (or refractory) to treatment with a CDK4/6 inhibitor if the cancer progresses during treatment without demonstrating any significant response or stabilization. Resistance may also be determined by a treating physician when the CDK4/6 inhibitor is no longer considered to be an effective treatment for the cancer.

A covalent CDK7 inhibitor (e.g., a compound of Formula A, Compound 1 or a pharmaceutically acceptable salt thereof) and any second therapeutic agent utilized in the methods described herein can be included in a kit and/or formulated in a pharmaceutically acceptable composition that includes the first agent, the second agent, and a pharmaceutically acceptable carrier.

A covalent CDK7 inhibitor (e.g., Compound 1) and any second therapeutic agent utilized in the methods of the present disclosure can be prepared and administered in a wide variety of oral or parenteral dosage forms. Thus, these agents can be administered by injection (e.g. intravenously, intramuscularly, intracutaneously, subcutaneously, intraduodenally, or intraperitoneally). Alternatively, Compound 1 and any second therapeutic agent can be administered by inhalation, for example, intranasally. Additionally, Compound 1 and any second therapeutic agent can be administered transdermally. It is also envisioned that multiple routes of administration (e.g., intramuscular, oral, transdermal) can be used to administer one or both of Compound 1 and the second therapeutic agent.

For preparing pharmaceutical compositions including a compound described herein, pharmaceutically acceptable excipients can be added in either solid or liquid form or a combination thereof. Solid form preparations within the scope of the present invention include powders, tablets, pills, capsules, cachets, suppositories, and dispersible granules. A solid carrier can be a substance that may also act as a diluent, flavoring agent, binder, preservative, tablet disintegrating agent, or encapsulating material. In powders, the excipient (e.g., a carrier) is a finely divided solid in a mixture with the finely divided active component (e.g., a compound described herein). In tablets, the active component (e.g., a compound described herein) is mixed with the excipient having the necessary binding properties in suitable proportions and compacted in the shape and size desired. Pharmaceutical compositions, including those formulated as powders and tablets, can contain from 5% to 70% of the active compound (i.e., a compound described herein). Suitable excipients (e.g., carriers) are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation,” when used in connection with a pharmaceutical composition, is intended to include, but is not limited to, the formulation of an active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In some embodiments, suitable carriers for parenteral administration will be selected for human administration. In particular, carriers for parenteral administration include aqueous solutions of dextrose, saline, pure water, ethanol, glycerol, glycerol formal, polyethylene glycol, propylene glycol, peanut oil, sesame oil, polyoxyethylene-block polymers, pyrrolidine, N-methyl pyrrolidione, and the like. Ampoules are convenient unit dosages. The compounds of the present disclosure can also be incorporated into liposomes or administered via transdermal pumps or patches. Pharmaceutical admixtures suitable for use in the present disclosure include those described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical compositions can be in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be similar to that being utilized in a human clinical trial or, for those agents that have already been approved for use, the dosages indicated on the prescribing information for that agent (or dosages below those described in the prescribing information where a synergistic effect is attained).

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

Pharmaceutical compositions of the present invention may additionally include components to provide sustained release and/or comfort (e.g., high molecular weight, anionic mucomimetic polymers, gelling polysaccharides, and finely-divided drug carrier substrates). These components are discussed in greater detail in U.S. Pat. Nos. 4,911,920; 5,403,841; 5,212,162; and 4,861,760. The entire contents of these patents are incorporated herein by reference in their entirety for all purposes.

For many of the agents described herein, effective dosage forms are known in the art.

A covalent CDK7 inhibitor described herein, (e.g., Compound 1) can be formulated into an aqueous pharmaceutical composition comprising sulfobutyl ether-β-cyclodextrin (SBEβCD), such as Captisol® and/or formulated into a dosage form for intravenous infusion.

Pharmaceutical compositions utilized in the present disclosure include compositions wherein the active ingredient(s) is contained in a therapeutically effective amount, i.e., in an amount effective to achieve its intended purpose. The actual amount effective for a particular application will depend, inter alia, on the condition being treated. For example, when administered in methods to a subject with cancer, such compositions will contain an amount of active ingredient effective to achieve the desired result.

The dosage and frequency (single or multiple doses) of compound administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds of the present disclosure.

For any compound or pharmaceutical composition described herein, the therapeutically effective amount can be initially determined from, or informed by data generated in, cell culture assays and/or animal models of disease. For example, a dose for humans can be formulated to achieve a concentration that has been found to be effective in animals. The dosage in humans can be adjusted by, for example, monitoring kinase inhibition, other markers, the signs and symptoms of the disease being treated, and side effects and subsequently adjusting the dosage upwards or downwards.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

For many of the agents described herein, effective dosage amounts and intervals are known in the art. Such dosage amounts and intervals can be adjusted individually to provide levels of the administered compound(s) effective for the particular clinical indication being treated. This will provide a therapeutic regimen that is commensurate with the severity of the individual's disease state.

In one embodiment, the amount of the covalent CDK7 inhibitor (e.g., Compound 1 or a compound of Formula A) to be administered is between about 1-500 mg/m² administered once or twice per week. For example, the amount to be administered can be between 2-128 mg/m² once or twice a week via intravenous infusion.

In certain embodiments, the invention provides the use of a combination of Compound 1 and a second therapeutic agent selected from a) a Bcl-2 inhibitor, b) a CDK9 inhibitor, c) a Flt3 inhibitor, d) a PARP inhibitor, e) a BET inhibitor, f) a CDK4/6 inhibitor, g) a platinum-based anti-cancer agent, or h) a taxane to treat a subject suffering from a disease (e.g., a cancer; e.g., a specific type of cancer, e.g., a breast cancer or an ovarian cancer). Inhibitors in each of these classes of second therapeutic agents are well-known in the art. In some aspects of these embodiments, the subject to be treated is naïve (e.g., has not been exposed to) the second therapeutic agents. In alternate aspects of these embodiments, the subject to be treated has been exposed to and has demonstrated resistance or is refractory to the second therapeutic agent when administered as a monotherapy.

Examples of useful Bcl-2 inhibitors include, but are not limited to, venetoclax, APG-1252, S55746, BP1002, and APG-2575.

Examples of useful CDK9 inhibitors include, but are not limited to, alvocidib, seliciclib (CYC202), AT7519, TG02, CYC065, BAY1251152, BAY 1143572, voruciclib (formerly P1446A-05), TP-1287, AZD5576, NVP2, nanoflavopiridol, and VS2-370.

Examples of useful Flt3 inhibitors include, but are not limited to, Rydapt® (midostaurin), Quizartinib, Pexidartinib/PLX3397, gilteritinib (ASP2215), Crenolanib besylate, Nexavar® (sorafenib), CDX-301, Iclusig® (ponatinib), pacritinib, SEL24, ENMD-2076, FF-10101-01, CT053PTSA, SKI-G-801, SKLB1028, FLYSYN, NMS-088, CG′806, and HM43239.

Examples of useful PARP inhibitors include, but are not limited to, Lynpraza® (olaparib), Zejula® (niraparib), Rubraca® (rucaparib), veliparib, talazoparib, 2x-121, CK-102, BGB-290, NT-125, and NMS-P293.

Examples of useful BET inhibitors include, but are not limited to, JQ1, GS-5829, FT-1101, ZEN-3694, GSK-2820151, I-BET762, GSK525762, CPI-0610, OTX015, I-BET151, CPI203, PFI-1, MS436, RVX2135, BAY1238097, INCB054329, TEN-010, BAY-299, BMS-986158, ABBV-075, and PLX51107.

Examples of useful CDK4/6 inhibitors include, but are not limited to, Ibrance® (palbociclib), Kisqali® (ribociclib), Verzenio® (abemaciclib), trilaciclib, G1T38, BPI-1178, and ON 123300.

Examples of useful platinum-based anti-cancer agents include cisplatin, carboplatin, oxaliplatin, nedaplatin, triplatin tetranitrate, phenanthriplatin, picoplatin, and satraplatin.

Examples of useful taxanes include Cremophor EL-paclitaxel (Taxol®), nab-paclitaxel (Abraxane®), and docetaxel (Taxotere®).

Unless otherwise specified, when employing a combination of a covalent CDK7 inhibitor (e.g., Compound 1 or a compound of Formula (A)) and a second therapeutic agent in a method of the invention, the second therapeutic agent can be administered concurrently with, prior to, or subsequent to the covalent CDK7 inhibitor. The second therapeutic pharmaceutical agent may be administered at a dose and/or on a time schedule determined for that pharmaceutical agent. The second therapeutic agent may also be administered together with the covalent CDK7 inhibitor (e.g., Compound 1) in a single dosage form or administered separately in different dosage forms. In general, it is expected that the second therapeutic agents utilized in combination with Compound 1 will be utilized at levels that do not exceed the levels at which they are utilized individually. In some embodiments, the levels of the second therapeutic agent utilized in combination will be lower than those utilized in a monotherapy due to synergistic effects.

For combinations of a covalent CDK7 inhibitor (e.g., Compound 1 or a compound of Formula A) and a second therapeutic agent, a kit comprising each of the two active therapeutics can be provided. In some instances, each of Compound 1 and the second therapeutic agent will be in separate vessels. In some instances, the kit includes a written insert or label with instructions to use the two therapeutics in a subject suffering from a cancer (e.g., as described herein). The instructions may be adhered or otherwise attached to a vessel or vessels comprising the therapeutic agents. Alternatively, the instructions and the vessel(s) can be separate from one another but present together in a single kit, package, box, or other type of container.

The instructions in the kit will typically be mandated or recommended by a governmental agency approving the therapeutic use of the combination. The instructions may optionally comprise dosing information for each therapeutic agent, the types of cancer for which treatment of the combination was approved or is intended, physicochemical information about each of the therapeutics, pharmacokinetic information about each of the therapeutics, drug-drug interaction information, or diagnostic information (e.g., based on a biomarker described herein).

### EXAMPLES

### Example 1. Correlating Compound 1 Sensitivity, Gene Expression and/or SE Strength

To identify potential biomarkers predictive of sensitivity to Compound 1, we evaluated the inhibitory activity of Compound 1 in a large panel of human tumor cell lines and correlated activity with RNA expression and epigenetic profiling data.

A panel of 406 human tumor cell lines (Chempartner), including 19 TNBC cell lines, were tested for response to various concentrations of Compound 1 (0.0005, 0.0015, 0.0046, 0.014, 0.041, 0.12, 0.37, 1.1, 3.3, and 10 uM) using the ATP-lite assay. Cell line growth was measured before treatment on Day 0 and then after treatment either after a minimum of 3 or maximum of 6 days depending on the cell-line. Simultaneously, the signal was for the same cell-line treated with DMSO for the same number of days as a negative control.

Clustering of growth-rate adjusted dose response curves of cell-lines treated with Compound 1 allowed the classification of cell-lines into low and high response groups. Based on the assay values, we computed normalized growth rate inhibition values at each concentration of Compound 1 by comparing growth rates in the presence and absence of that drug and then fit the results to a sigmoidal curve using known methods (Hafner et al., Nature Methods, 2016).

Fitting of the growth rate inhibition curves (GR curves) enabled measurement of various metrics such as GRmax (the maximum GR at the highest concentration of drug tested), and GRO (the concentration of drug at which the GR value crossed 0 (transitioned from cytostatic to cytotoxic)).

We next assigned each cell line to either the “high” or “low” categories based on response to Compound 1. We developed the following algorithm for this purpose. We assigned two sets of seeds: a low responder group of cell-lines all with GRmax>0 (e.g., cell lines for which Compound 1 was not cytotoxic even at the highest concentration tested), and a high responder group with GRmax<−0.5 and GRO<100 nM (e.g., cell lines for which Compound 1 was highly cytotoxic at a concentration of <100 nM). The goal of this algorithm was to classify all the remaining cell-lines that did not fall into one of these two groups as “low” or “high” responders, while allowing for some flexibility in reassigning the seeds to a different group. Using pairwise Euclidean distance between the theoretical GR curves for all cell-lines, we performed hierarchical clustering with Ward.D2 linkage 1000 times (clusterings), using a random subset of 90% of the cell lines each time. For each clustering, a cell line was counted as “high” if its curve was closer to high response seed and “low” if its curve was closer to low response seed in that particular clustering iteration. We then determined the % of clustering iterations that the cell line was counted as “high” and “low”. If the cell line was counted as “low” in over 50% of the iterations, it was classified as a “low” responder. If the cell line was counted as “high” in over 50% of the iterations, it was classified as being a “high” responder.

For all cell lines assigned to low and high responder categories, we identified those that had matching mRNA expression data, either microarray or RNA-Seq, from Cancer Cell Line Encyclopedia (CCLE). We identified 18,074 genes with both microarray and RNA-seq data available. For each gene and each cell line mRNA dataset, we built a linear classifier to predict whether the cell line belonged to the low responder class or high responder class based on the mRNA expression level of only that gene. The classifier used was the linear discriminant analysis from the MASS package in R and the assignment of each cell line was tested using leave-one-out cross validation. True positive rate was calculated as the number of high responder cell-lines that were accurately classified as such based solely on mRNA expression, while true negative rate was calculated as the number of low responder cell lines that were accurately classified as such based solely on mRNA expression. “Accuracy” was measured as the average of true positive rate and true negative rate. False Discovery Rate (FDR) was calculated by randomly permuting “low” and “high” responder labels across cell-lines 1000 times, and then calculating the accuracy for each iteration using the linear classifier-based approach described above. We then approximated the distribution of random balanced accuracies per gene as a normal distribution. FDR was calculated as the probability of observing an accuracy value from the random distribution that was higher than the real predicted accuracy for that gene. The lower the FDR, the higher our confidence would be that the gene expression was truly predictive of cell-line response. Typically, an accuracy of at least 65% combined with an FDR<0.05 indicated that expression of a particular gene was predictive of sensitivity to Compound 1. Higher FDRs required higher accuracy to find a correlation between mRNA expression (or super-enhancer strength) sensitivity to Compound 1.

SE analysis was performed using genome-wide H3K27Ac scores based on the SE score algorithm of McKeown et al. (Cancer Discovery, 7(10):1136-1153, 2017). H3K27ac scores per SE were used to calculate accuracy and FDR in the same way as described above for expression per gene. Copy number data was obtained from CCLE.

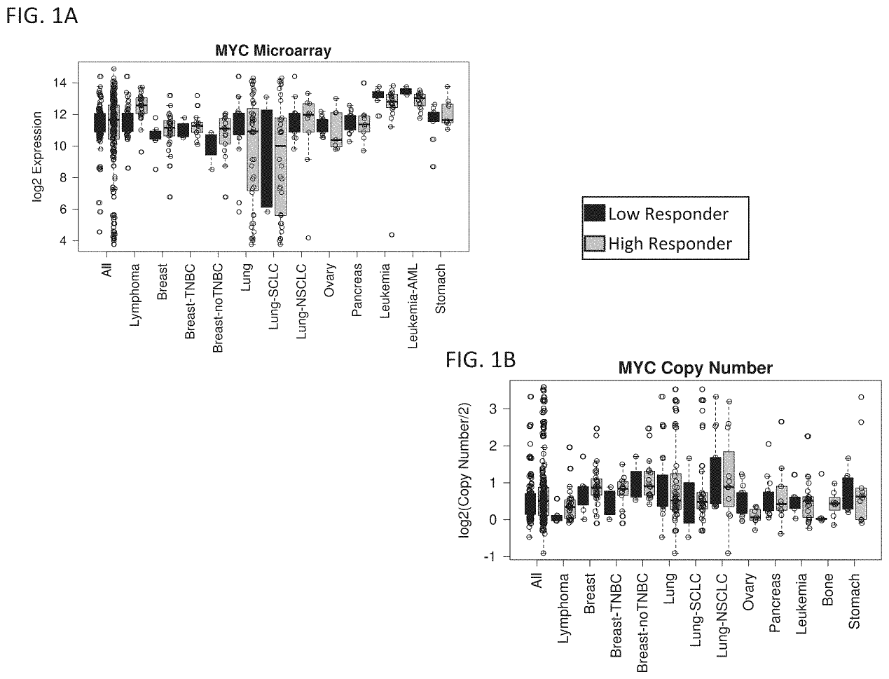

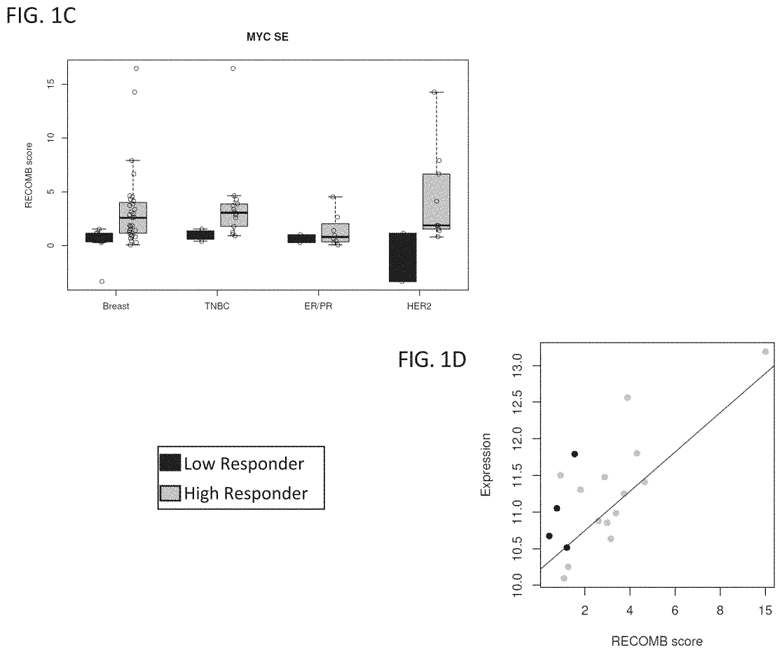

For the MYC CNV plot (FIG. 1B), we built a linear classifier using the CNV values for the MYC gene to predict the “low” or “high” responder class for cell-lines as described above.

Twenty-five genes were differentially expressed between Compound 1-sensitive and -insensitive tumor lines (FDR<0.05 and/or accuracy>65%). The cell lines were further divided by cancer type and/or subtype to determine if the differential expression of these genes was significant in determining sensitivity to Compound 1. Of the 25 genes, mRNA expression levels of at least some of them predicted the response to Compound 1 in at least a subset of cancers.

For MYC, there was no significant correlation between mRNA expression and response to Compound 1 in the majority of cancer subtypes (FIG. 1A), nor was there any significant correlation between MYC copy number and sensitivity (FIG. 1B). A significant correlation was found, however, between MYC-associated super enhancer strength (as determined by SE score) and sensitivity to Compound 1 in breast cancer samples tested and was also seen in both TNBC and non-TNBC sample subsets (FIG. 1C). Moreover, further analysis of TNBC cells demonstrated that there was a correlation between MYC-associated super enhancer strength and MYC expression as shown in FIG. 1D.

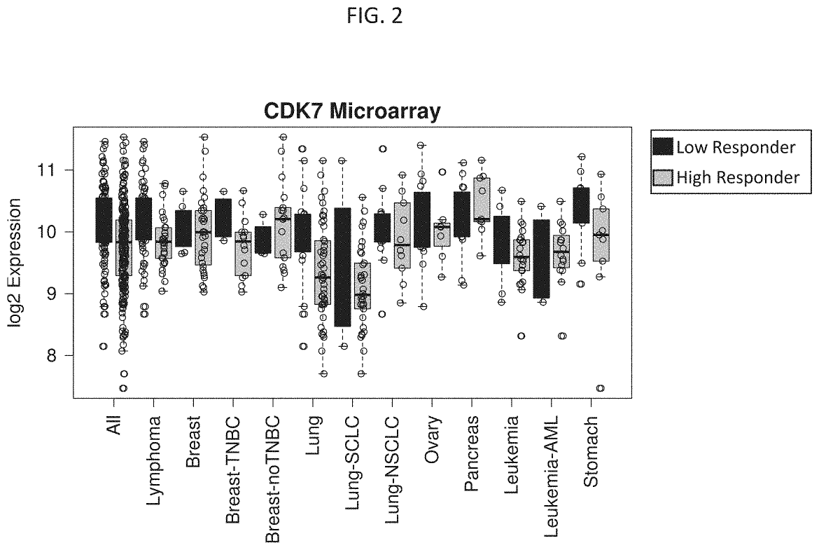

For CDK7, we found a significant inverse correlation between CDK7 mRNA levels and sensitivity to Compound 1 in all samples and in lymphoma. We also observed an inverse correlation between CDK7 mRNA levels and sensitivity to Compound 1 in lung cancer, leukemia, and stomach cancer cell lines (FIG. 2).

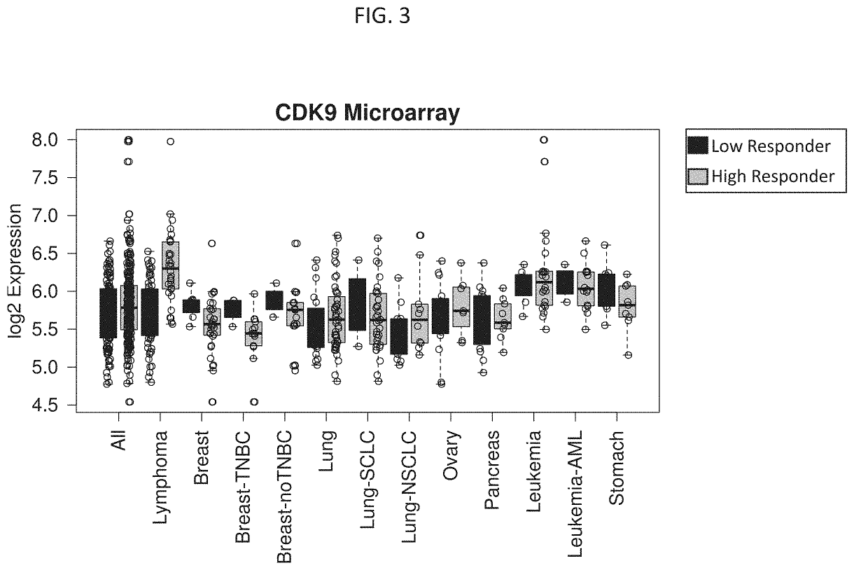

For CDK9, we found a significant inverse correlation between mRNA levels and sensitivity to Compound 1 in all breast and TNBC cell lines (FIG. 3).

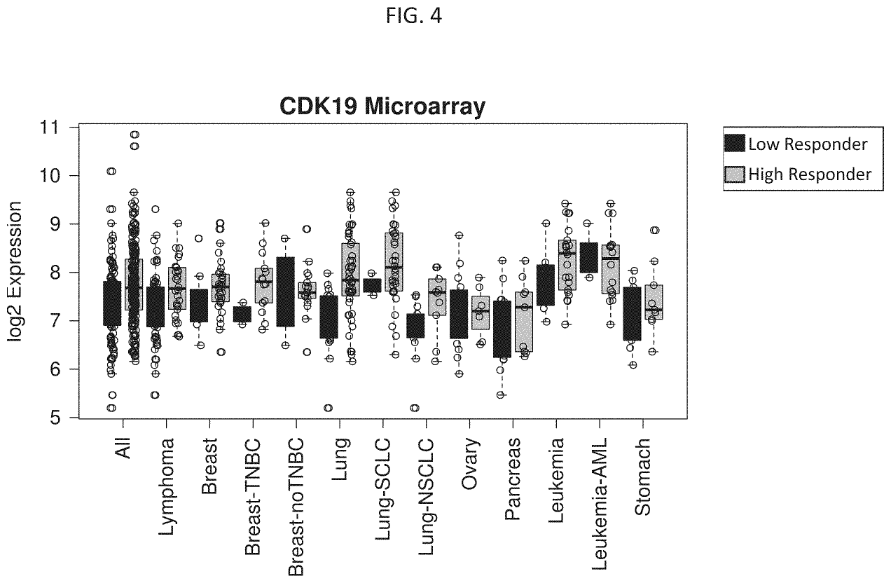

For CDK19, we found a significant correlation between mRNA levels and sensitivity to Compound 1 in all, all breast cancer, TNBC, all lung, small cell lung and non-small cell lung cancer cell lines (FIG. 4).

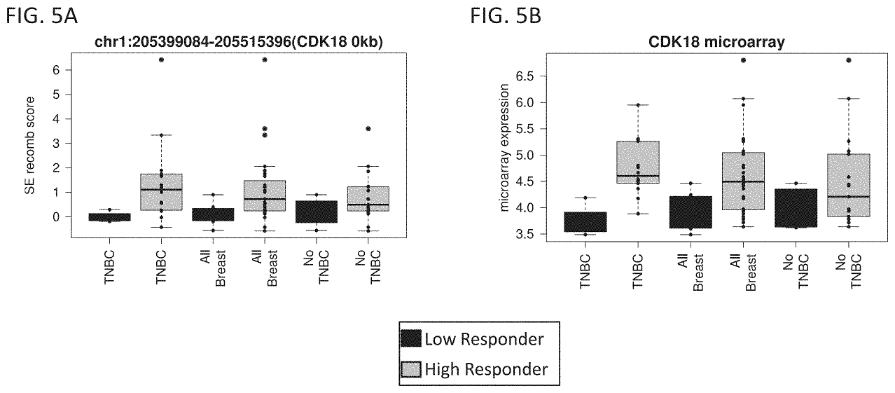

For CDK18, we found a significant correlation between super enhancer strength (FIG. 5A) and sensitivity to Compound 1 in all breast and TNBC cell lines. We also found a significant correlation between mRNA levels and sensitivity to Compound 1 in all breast and TNBC cell lines (FIG. 5B).

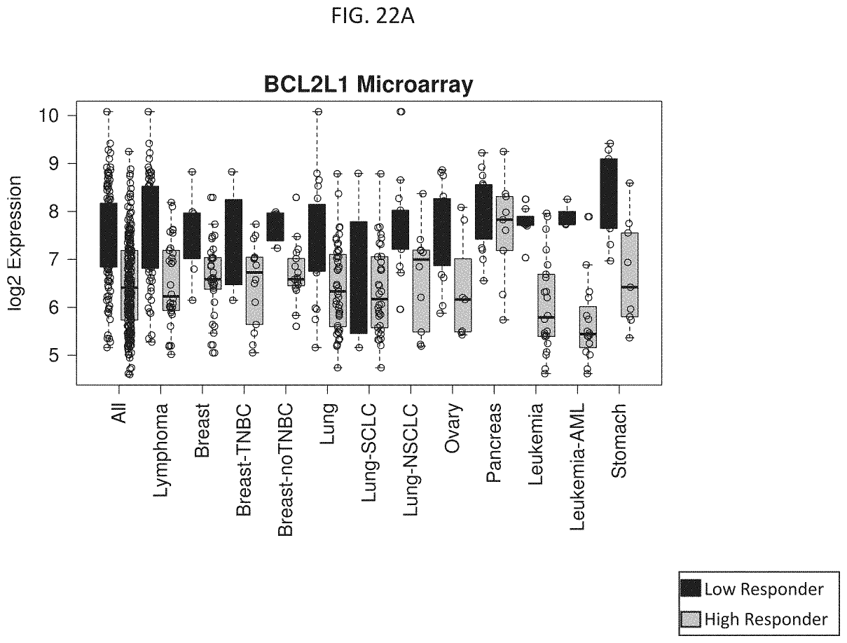

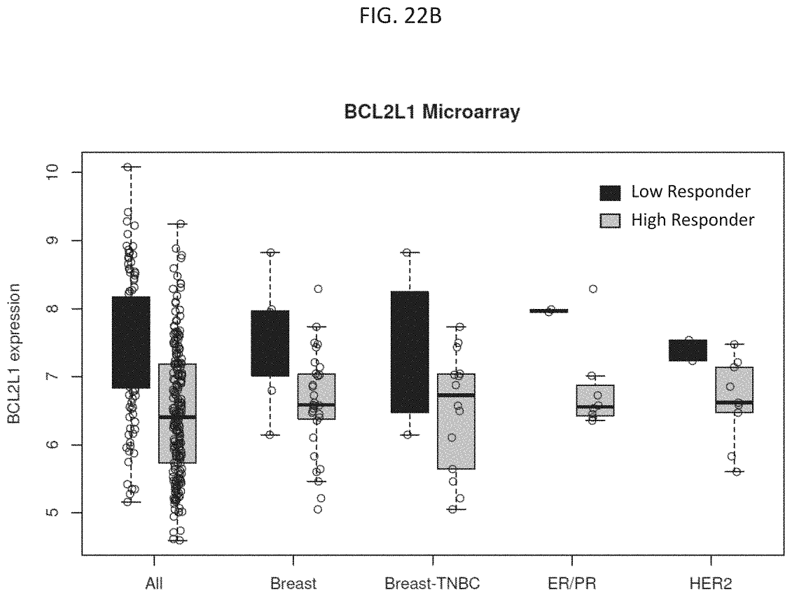

For BCL2L1, we found a significant inverse correlation between mRNA levels and sensitivity to Compound 1 in all cancer, all breast cancer (Accuracy=73%; FDR=0.055), TNBC, non-TNBC, ER⁺/PR⁺ breast cancer, HER2⁺ breast cancer, all lung cancer, NSCLC, ovarian, leukemia and stomach cancer cell lines (FIGS. 22A and 22B). Thus, lower expression of BCL2L1, which encodes the mitochondrial apoptosis regulator BCL-XL, was identified as the most predictive expression biomarker of sensitivity across all profiled cell lines, strongly separating the two classes of sensitivity.

The table below shows both the Accuracy and FDR values for each biomarker and each type of cancer cell line tested against Compound 1.

TABLE 1
Accuracy and FDR Values for Various Biomarker Gene Expression in Various Cancer
Cell Line Types Treated with Compound 1.

| Indication | Statistic | MYC | CDK7 | CDK9 | CDK18 | CDK19 | BCL2L1 |
|---|---|---|---|---|---|---|---|
| All | Accuracy (%) | 49 | 62 | 52 | 55 | 61 | 70 |
| | FDR | 0.52 | 0.051 | 0.39 | 0.24 | 0.075 | 0.0061 |
| Lymphoma | Accuracy (%) | 58 | 82 | 63 | 37 | 63 | 48 |
| | FDR | 0.33 | 0.028 | 0.2 | 0.77 | 0.21 | 0.55 |
| Breast (all) | Accuracy (%) | 65 | 42 | 71 | 70 | 70 | 76 |
| | FDR | 0.15 | 0.69 | 0.076 | 0.093 | 0.084 | 0.043 |
| TNBC | Accuracy (%) | 66 | 57 | 73 | 80 | 82 | 54 |
| | FDR | 0.21 | 0.33 | 0.1 | 0.051 | 0.058 | 0.41 |
| Breast, | Accuracy (%) | 48 | 67 | 70 | 49 | 18 | 79 |
| Non-TNBC | FDR | 0.54 | 0.18 | 0.14 | 0.53 | 0.95 | 0.076 |
| Lung (All) | Accuracy (%) | 63 | 69 | 53 | 59 | 72 | 65 |
| | FDR | 0.15 | 0.064 | 0.4 | 0.21 | 0.029 | 0.12 |
| Small Cell | Accuracy (%) | 0 | 43 | 43 | 64 | 65 | 43 |
| Lung | FDR | 0.99 | 0.59 | 0.62 | 0.21 | 0.19 | 0.63 |
| Non-Small | Accuracy (%) | 42 | 55 | 57 | 12 | 71 | 64 |
| Cell Lung | FDR | 0.7 | 0.36 | 0.32 | 0.99 | 0.099 | 0.16 |
| Ovary | Accuracy (%) | 54 | 61 | 60 | 66 | 33 | 73 |
| | FDR | 0.41 | 0.24 | 0.26 | 0.18 | 0.83 | 0.1 |
| Pancreas | Accuracy (%) | 48 | 47 | 42 | 71 | 56 | 53 |
| | FDR | 0.55 | 0.55 | 0.7 | 0.12 | 0.38 | 0.43 |
| Leukemia | Accuracy (%) | 68 | 63 | 54 | 51 | 58 | 89 |
| | FDR | 0.099 | 0.22 | 0.39 | 0.45 | 0.31 | 0.015 |
| AML | Accuracy (%) | 81 | 28 | 41 | 3 | 25 | 94 |
| | FDR | 0.05 | 0.86 | 0.66 | 0.98 | 0.87 | 0.014 |
| Stomach | Accuracy (%) | 50 | 72 | 67 | 61 | 50 | 72 |
| | FDR | 0.49 | 0.11 | 0.19 | 0.23 | 0.49 | 0.13 |

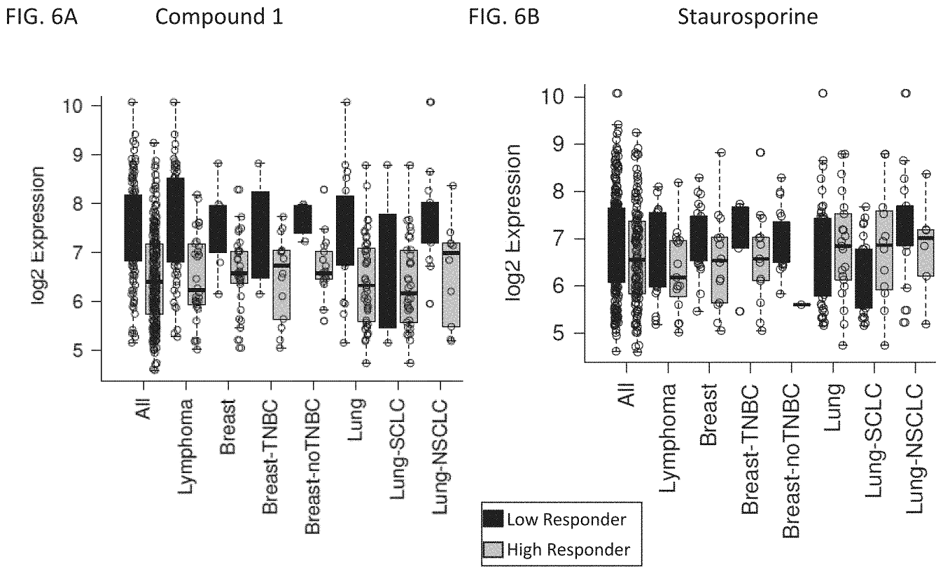

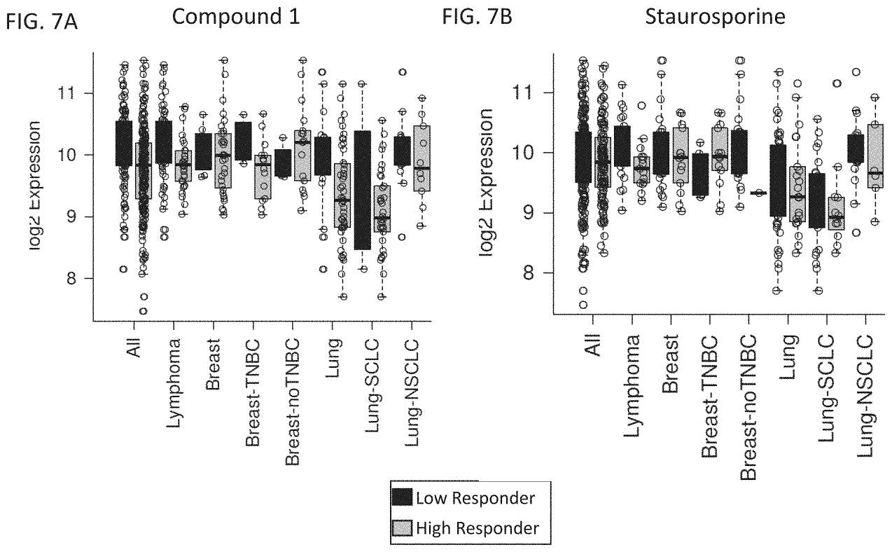

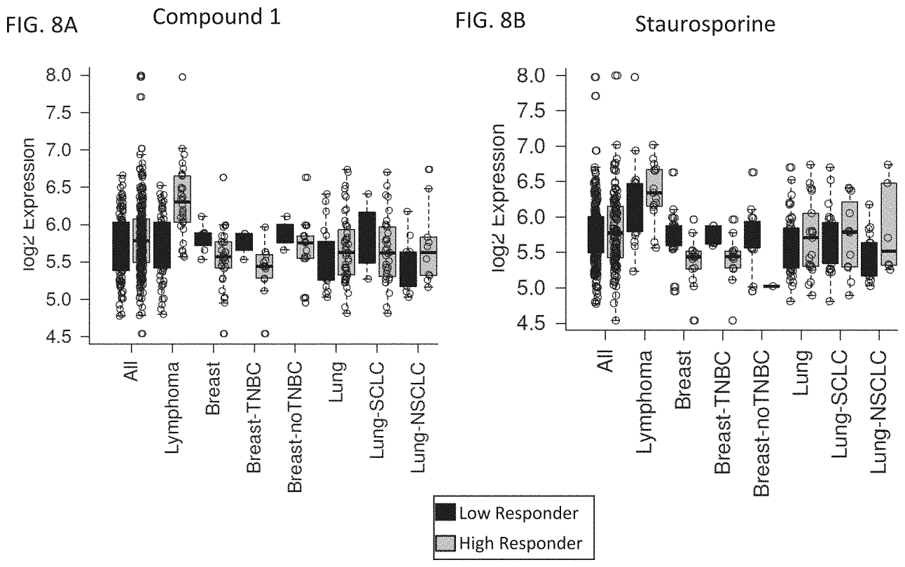

To confirm that sensitivity to Compound 1 was not simply due to some general toxic effect on the cells, we tested the sensitivity of these cell lines to the general kinase inhibitor staurosporine and queried if there was a correlation between sensitivity and various of the above-described biomarker mRNA level. We found no correlation between staurosporine sensitivity and BCL-XL expression (FIG. 6). We found some inverse correlation between staurosporine sensitivity and CDK7 expression, but not as strong as between Compound 1 sensitivity and CDK7 expression (FIG. 7). For CDK9, we found a strong correlation between mRNA level and staurosporine sensitivity in TNBC, just as we found for Compound 1 (FIG. 8). These results demonstrate that the correlations and inverse correlations found between sensitivity to Compound 1 and expression of various biomarkers is specific to the mechanism of action of Compound 1 and not due to a toxic effect of that compound on the cell lines.

The table below shows both the Accuracy and FDR values for each biomarker and each type of cancer cell line tested against staurosporine.

TABLE 2
Accuracy and FDR Values for Various Biomarker Gene
Expression in Various Cancer Cell Line Types.

| Indication | Statistic | BCL2L1 | CDK7 | CDK9 |
|---|---|---|---|---|
| All | Accuracy (%) | 54 | 55 | 50 |
| | FDR | 0.34 | 0.16 | 0.52 |
| Lymphoma | Accuracy (%) | 49 | 63 | 56 |
| | FDR | 0.49 | 0.16 | 0.35 |
| Breast (all) | Accuracy (%) | 57 | 57 | 85 |
| | FDR | 0.28 | 0.29 | 0.0083 |
| Triple-negative | Accuracy (%) | 67 | 51 | 92 |
| Breast | FDR | 0.2 | 0.42 | 0.0068 |
| Breast, Not TNBC | Accuracy (%) | 48 | 32 | 45 |
| | FDR | 0.016 | 0.5 | 0.08 |
| Lung (All) | Accuracy (%) | 53 | 57 | 56 |
| | FDR | 0.36 | 0.28 | 0.33 |
| Small Cell Lung | Accuracy (%) | 64 | 38 | 60 |
| | FDR | 0.22 | 0.75 | 0.26 |
| Non-Small Cell | Accuracy (%) | 61 | 61 | 58 |
| Lung | FDR | 0.2 | 0.17 | 0.3 |
| Pancreas | Accuracy (%) | 37 | 51 | 35 |
| | FDR | 0.77 | 0.39 | 0.76 |
| Leukemia | Accuracy (%) | 66 | 42 | 50 |
| | FDR | 0.18 | 0.68 | 0.5 |
| AML | Accuracy (%) | 70 | 60 | 55 |
| | FDR | 0.16 | 0.28 | 0.43 |
| Bone | Accuracy (%) | 71 | 65 | 44 |
| | FDR | 0.15 | 0.23 | 0.6 |
| Stomach | Accuracy (%) | 65 | 14 | 57 |
| | FDR | 0.22 | 0.97 | 0.34 |

The table below shows both the Accuracy and FDR values for MYC and CDK18 SEs in breast cancer cell lines tested against Compound 1.

TABLE 3
Accuracy and FDR Values for Various Biomarker Gene
Expression in Various Cancer Cell Line Types.

| | Accuracy | FDR | Accuracy | FDR |
|---|---|---|---|---|
| | TNBC | TNBC | All Breast | All Breast |
| MYC SE | 86% | 0.017 | 76% | 0.023 |
| CDK18 SE | 79% | 0.065 | 70% | 0.094 |

The table below shows both the Accuracy and FDR values for MYC copy number in cancer cell lines tested against Compound 1.

TABLE 4
Accuracy and FDR Values for the Presence of a MYC
Super-Enhancer in Various Cancer Cell Line Types.

| Indication | Statistic | MYC |
|---|---|---|
| Lymphoma | Accuracy (%) | 72 |
| | FDR | 0.08 |
| Breast (all) | Accuracy (%) | 60 |
| | FDR | 0.23 |
| TNBC | Accuracy (%) | 64 |
| | FDR | 0.22 |
| Breast, Not TNBC | Accuracy (%) | 46 |
| | FDR | 0.57 |
| Lung (All) | Accuracy (%) | 45 |
| | FDR | 0.63 |
| Small Cell Lung | Accuracy (%) | 54 |
| | FDR | 0.41 |
| Non-Small Cell | Accuracy (%) | 5 |
| Lung | FDR | 1 |
| Ovary | Accuracy (%) | 61 |
| | FDR | 0.27 |
| Pancreas | Accuracy (%) | 45 |
| | FDR | 0.63 |
| Leukemia | Accuracy (%) | 26 |
| | FDR | 0.94 |
| Bone | Accuracy (%) | 78 |
| | FDR | 0.09 |
| Stomach | Accuracy (%) | 28 |
| | FDR | 0.9 |

In these studies, we show for the first time that Compound 1 induced differential responses across a large panel of human tumor cell lines derived from multiple indications. We also show that, in this panel of cell lines, the response could be predicted in an “indication agnostic” manner by the level of expression of BCL2L1. Finally, in line with prior reports, in TNBC cell lines, MYC SE was significantly associated with sensitivity to Compound 1. These observations have generated strong hypotheses for selection strategies aimed at identifying patients with tumors particularly sensitive to CDK7 inhibition with Compound 1 and warrant further investigation with respect to predictive biomarkers of response in patients. Compound 1 is currently being assessed in a phase 1 trial in adult patients with advanced solid tumors, including a planned expansion cohort enriching for patients with TNBC (NCT03134638).

### Example 2. Compound 1 is Associated with Downregulation of MCL1 in Breast Cancer and Ovarian Cancer Cell Lines

The inverse correlation between efficacy of Compound 1 and BCL-XL mRNA level led us to hypothesize that Compound 1 was affecting the intrinsic apoptotic pathway of which BCL-XL is a part. It is known that three BCL2 family members, MCL1, BCL-XL and BCL2 are all involved in inhibiting apoptosis with somewhat redundant functions. Thus, we explored the effect of Compound 1 on each of these genes at both the mRNA and protein level in various cancer cell lines.

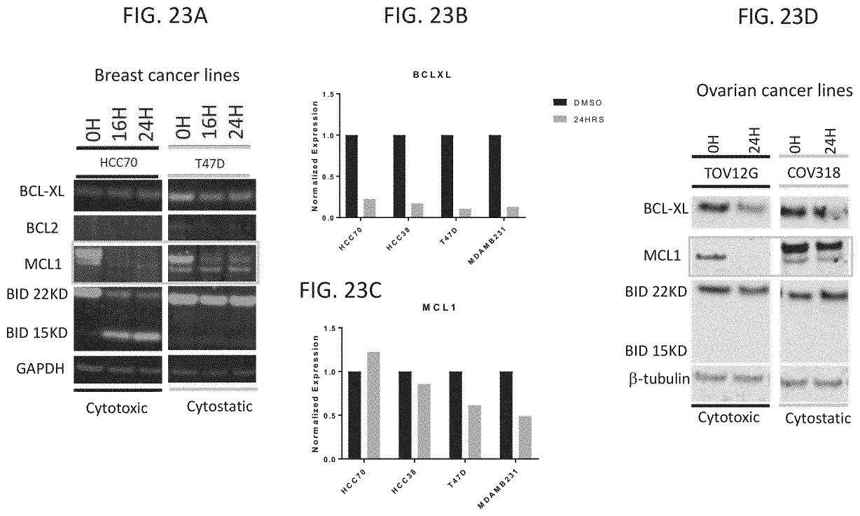

Cytotoxic cancer cell lines HCC70 and TOV21G, and cytostatic cancer cell lines T47D and COV318 were seeded in wells of a six-well dish at a density of 1×10⁶ cells/well and allowed to adhere overnight. Cells were then treated with 50 nM of Compound 1 for 16 or 24 hours, or with DMSO (0 hours) representing a negative control. Cells were harvested and placed on ice, and resuspended in RIPA lysis buffer supplemented with protease and phosphatase inhibitors. Clarified lysates prepared with 4×LSD running buffer and boiled at 95° C. for 5 minutes. Equivalent amounts of sample (10 μg total protein) were run on a 4-12% Bis-Tris gel and transferred to a PDVF membrane for Western blotting using standard Western blotting protocols. Membranes were blocked with Licor TBS blocking buffer then probed with antibodies for BCL-XL, BCL-2, MCL1, BID, and GAPDH visualized using and Odyssey imager (FIGS. 23A and 23D).

Breast cancer cell lines HCC70, HCC38, T47D, and MDAMB231 were plated in a 96-well plate at 20,000 cells/well and allowed to adhere overnight. Cells were then treated with 50 nM of Compound 1 or DMSO for 24 hours. Media was aspirated and mRNA isolated using Dynabeads™ mRNA DIRECT™ Purification Kit (Invitrogen) and amount of mRNA measured using qPCR with the TaqMan probes for the transcript of interest (either BCLXL or MCL1). Transcripts were quantified using AACt calculation, normalizing to the house keeping gene GAPDH (FIGS. 23B and 23C).

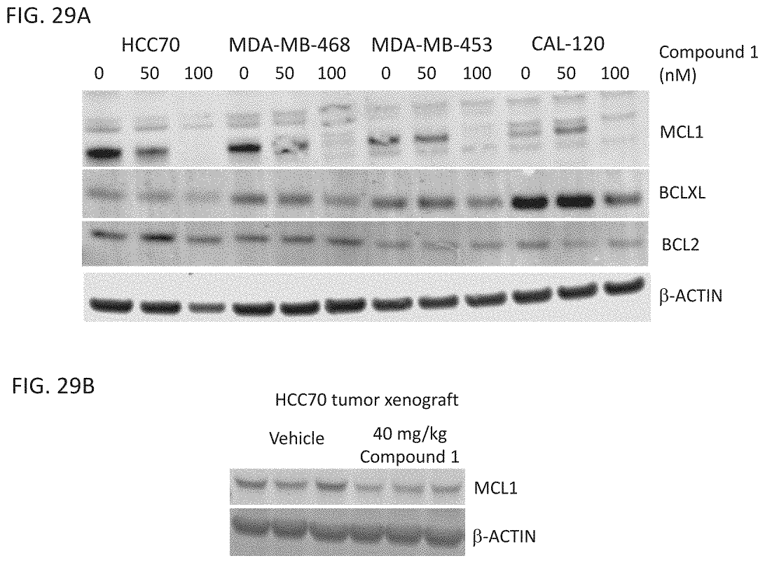

In a similar experiment, TNBC breast cancer cell lines HCC70, MDAMB468, MDAMB453 and CAL-120 were grown as described above and treated with DMSO, 50 nM Compound 1 or 100 nM Compound 1 for 24 hours. Protein samples were prepared as described above for Western blotting using antibodies specific for the indicated proteins in FIG. 29A. As shown in FIG. 29A, MCL1 was downregulated by Compound 1 treatment in all four cell lines, but more substantially in cell lines having low levels of BCLXL (HCC70 and MDAMB468) as compared to the cell line having substantially higher levels of BCLXL (CAL-120).

### Example 3. Compound 1 is Associated with Downregulation of MCL1 in AML Cell Lines Having Low BCL-XL and Higher BCL2

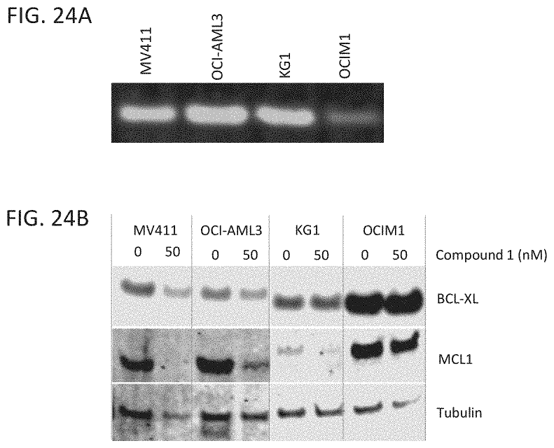

We next explored the expression of MCL1, BCL-XL and BCL2 in four different AML cell lines. MV411, OCI-AML3 and KG1 cells were separately grown and prepared as described in Example 2 for Western blotting, using antibodies specific for BCL2 (FIG. 24A), BCL-XL, MCL-1 and tubulin as an internal control (FIG. 24B).

As shown in FIG. 24A, BCL2 protein was robustly expressed in three of the tested AML cell lines (MV411, OCI-AML3 and KG1), but not in the fourth (OC1M1). Treatment of these four cell lines with 50 nM of Compound 1 resulted in significant reduction in MCL1 in MV411, OCI-AML3 and KG1, but little effect on that target in OC1M1 (FIG. 24B). Not surprisingly, OCIM1 expressed a high amount of BCL-XL compared to BCL-XL levels in the other three cell lines (FIG. 24B). These results seem to confirm our findings that high BCL-XL expression levels are correlated with low sensitivity to Compound 1 and suggest that such high expression levels may affect the ability of Compound 1 to decrease MCL1.

### Example 4. Exploration of Synergy Between Compound 1 and Other Cancer Therapeutics

The redundancy of the BCL2 family members' role as apoptosis inhibitors and our results showing the inverse correlation between the efficacy of Compound 1 and BCL-XL mRNA level suggested that effective treatment of certain cancers might require low levels of all three of BCL-XL, BCL2 and MCL1. We therefore examined the combined effect of Compound 1 and the known BCL2 inhibitor venetoclax on various cancer cell lines, as well as the combined effect of Compound 1 and other therapeutic agents.

Using a Biotek EL406, 50 μL of cell media containing 20-60,000 cells/ml was distributed into white 384-well Nunc plates (Thermo). Suspension cells then received compound immediately while adherent cells lines were given one hour to reattach to the surface of the plate prior to compound addition. Compound 1 and the second agents to be tested were dissolved in DMSO and arrayed on 384 well compound storage plates (Greiner). Each compound plate received Compound 1 and one second agent each in 5 different doses centered approximately on the EC₅₀ of the given compound for a given cell line, providing a total of 25 different dose combinations of the two agents.

Compound arrays were distributed to assay plates using a 20 nl 384-well pin transfer manifold on a Janus MDT workstation (Perkin Elmer). Each plate contained 8 replicates of all 5 by 5 compound concentrations in addition to five doses of each compound on its own in quadruplicate. After addition of compounds, cell plates were incubated for 5 days in a 37° C. incubator. Cell viability was evaluated using ATPlite (Perkin Elmer) following manufacturer protocols. Data was analyzed using commercially available CalcuSyn software and visualized using GraphPad Prism Software. Isobolograms plotting each of the 25-dose combination of Compound 1 and the second agents were generated and analyzed for the presence of synergy. In the isobolograms, the straight line connecting the abscissa and the ordinate values of 1.0 represents growth inhibitions that were additive for the combination of the two compounds. Plots that fall below the straight line represented synergistic growth inhibitions, with plots that fall below that line and one connecting the abscissa and the ordinate values of 0.75 represent mild synergy. Plots that fall between a line connecting the abscissa and the ordinate values of 0.75 and a line connecting the abscissa and the ordinate values of 0.25 represent moderate synergy. Plots that fall below a line connecting the abscissa and the ordinate values of 0.25 represent strong synergy. Data points outside the maxima in each isobologram are indicated by the number of asterisks at the top of the isobologram and represent data points of no synergy.

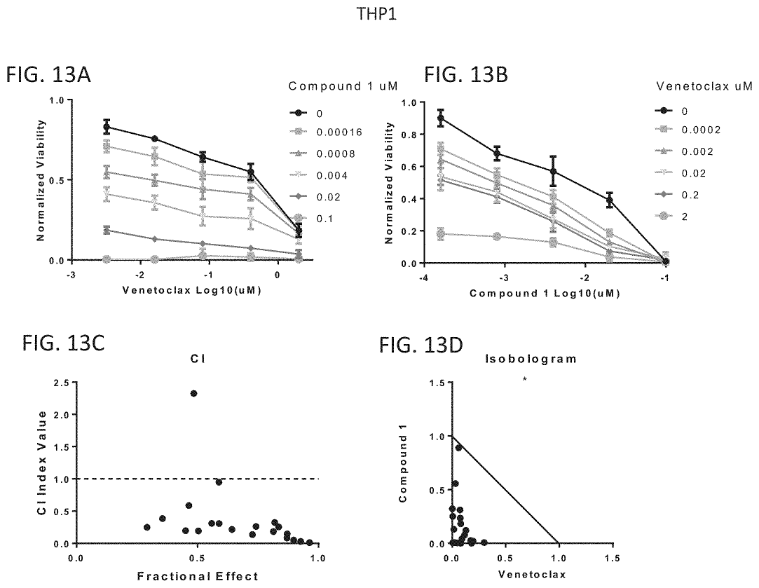

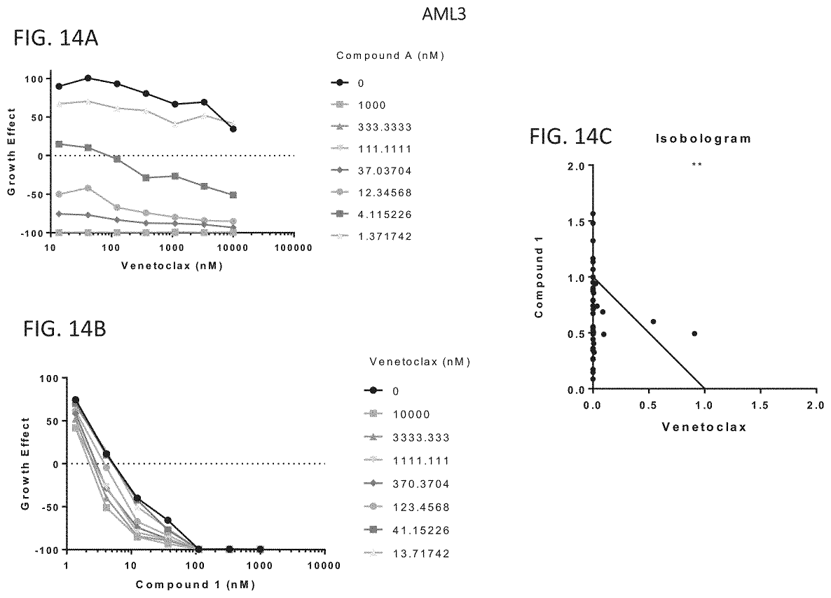

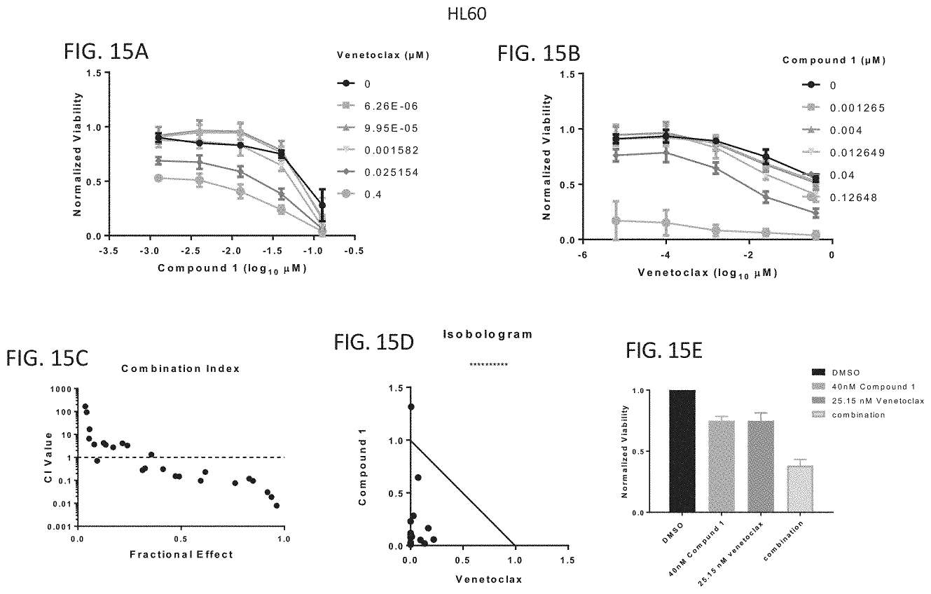

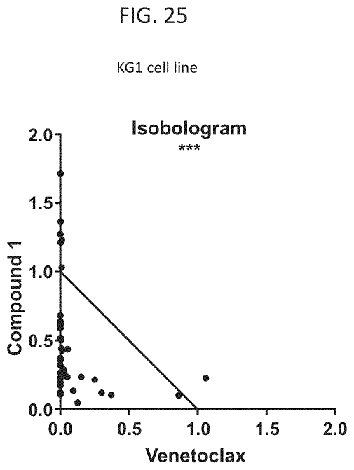

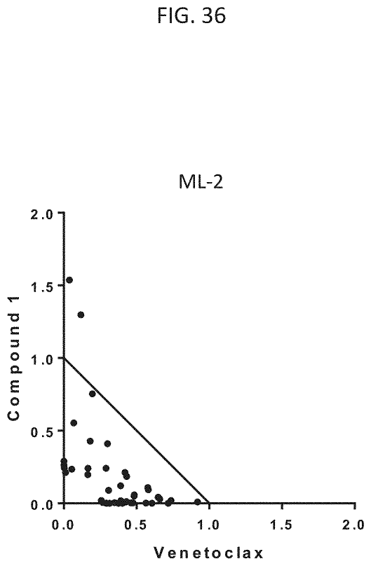

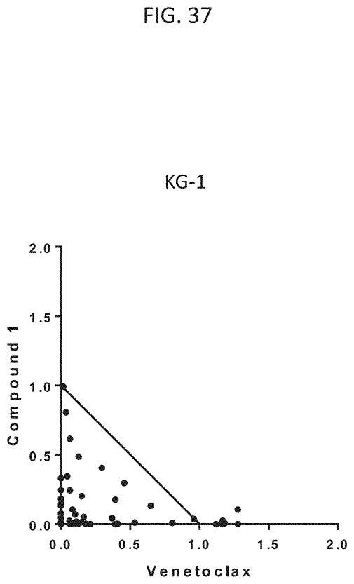

The combined effect of Compound 1 and venetoclax was examined on seven AML cell lines: THP1 (FIGS. 13A-13D), AML3 (FIGS. 14A-14C), HL60 (FIGS. 15A-15D), KG1 (FIG. 25A), ML-2 (FIG. 36), KG-1 (FIG. 37), and OCI-M1 (not shown). Synergy was shown for this combination in THP1, HL60, KG1, ML-2 and KG-1. OCI-M1 showed no synergy and is an AML cell line that is known to have a high expression of BCLXL.

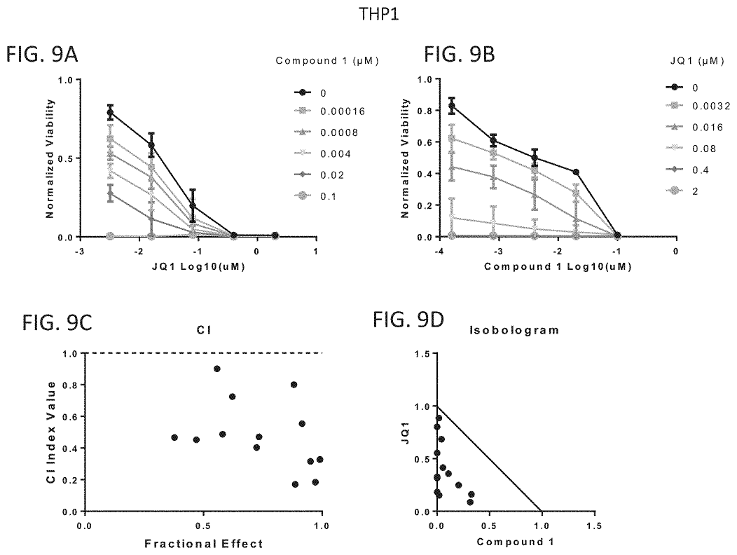

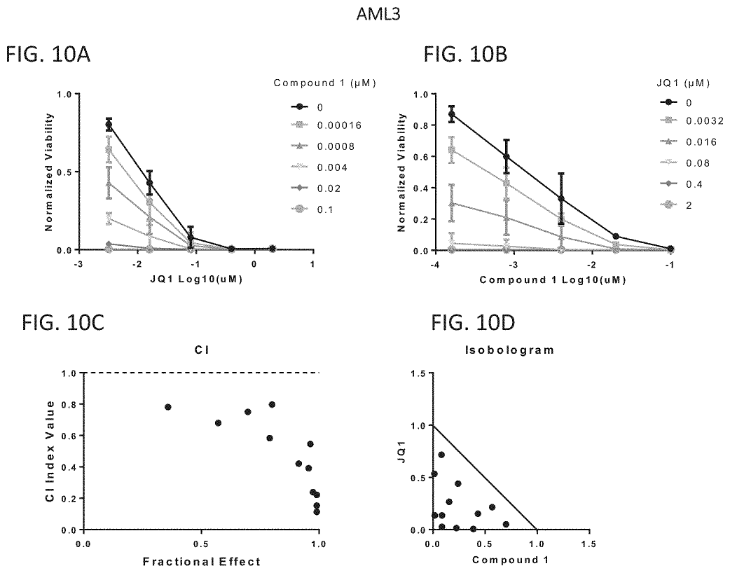

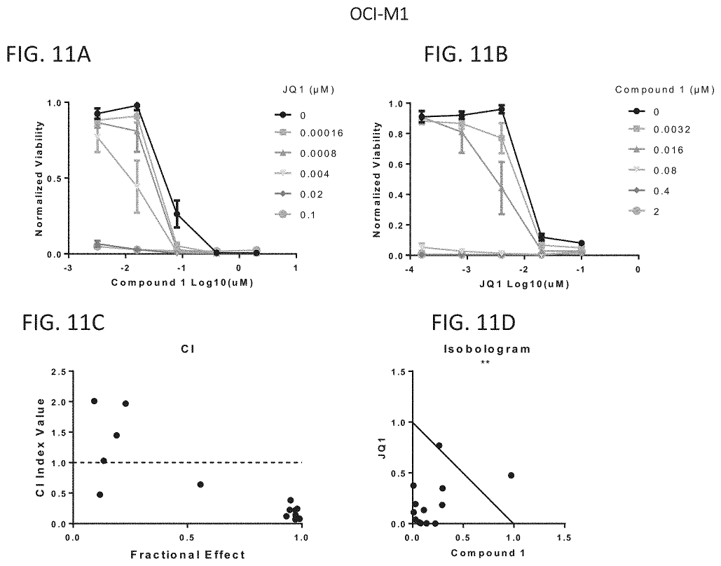

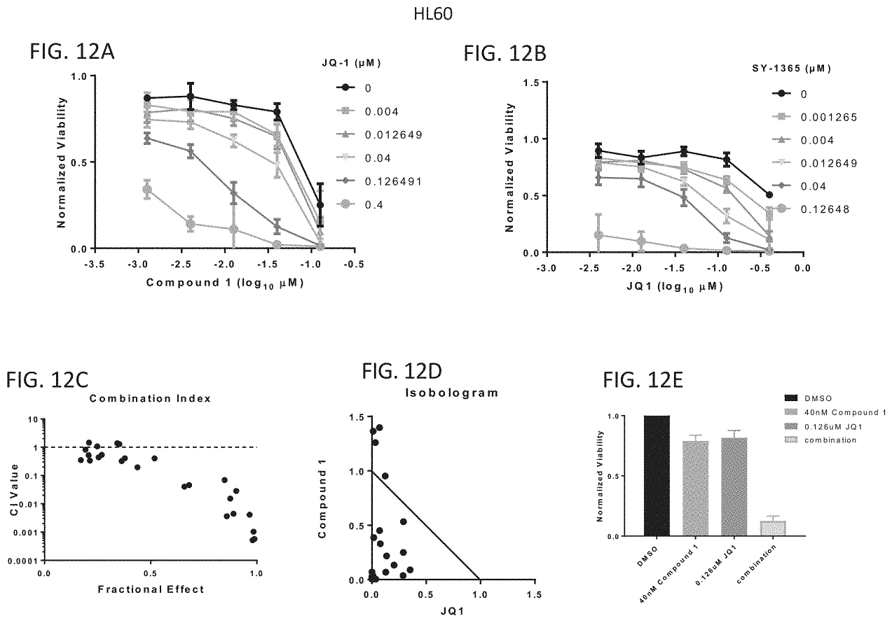

We also examined the combined effect of Compound 1 and the BET inhibitor JQ1 on four different AML cell lines: THP1 (FIGS. 9A-9D), AML3 (FIGS. 10A-10D), OCI-M1 (FIGS. 11A-11D) and HL60 (FIGS. 12A-12E). Synergy was observed for this combination in all four AML cell lines tested.

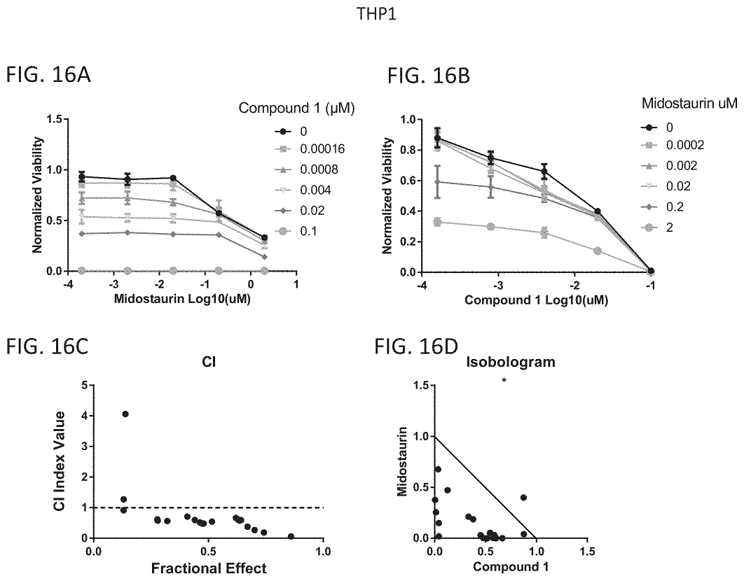

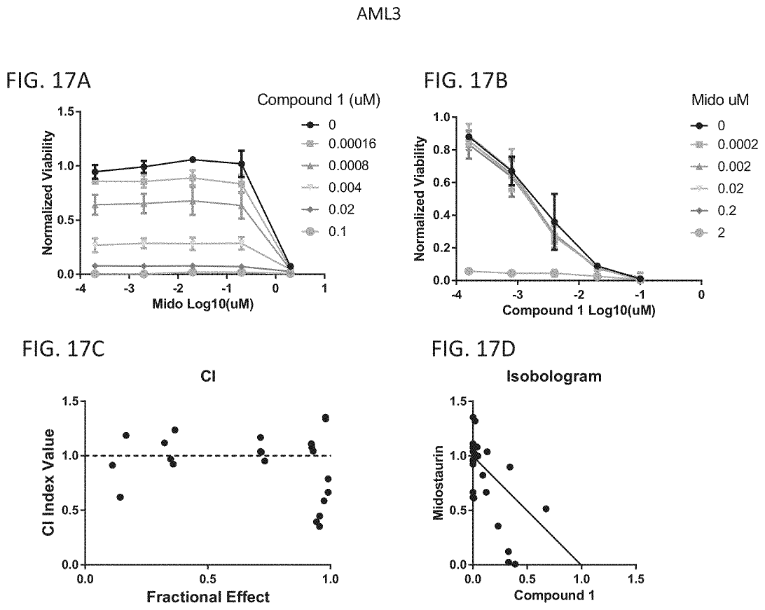

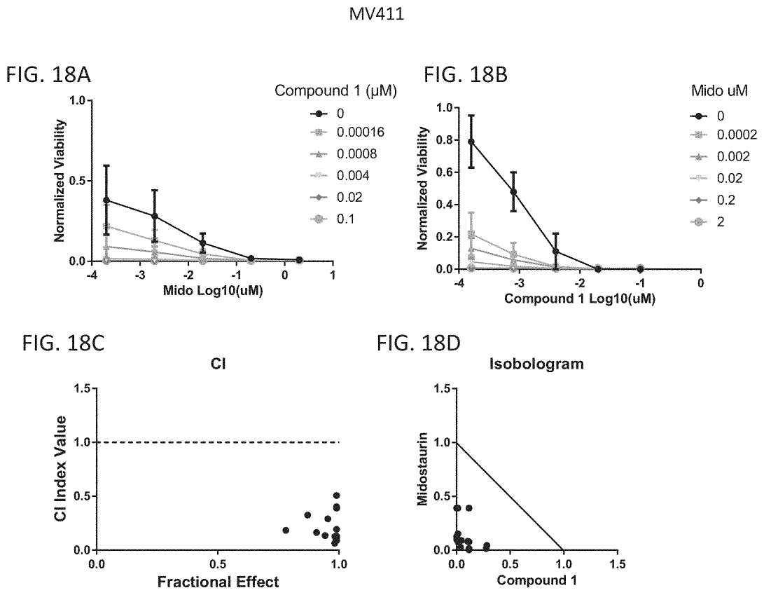

We examined the combined effect of Compound 1 and the FLT3 inhibitor midostaurin on three different AML cell lines: THP1 (FIGS. 16A-16D), AML3 (FIGS. 17A-17D), and MV411 (FIGS. 18A-18D). Synergy was observed for this combination in THP1 and MV411, while AML3 showed a mostly additive effect.

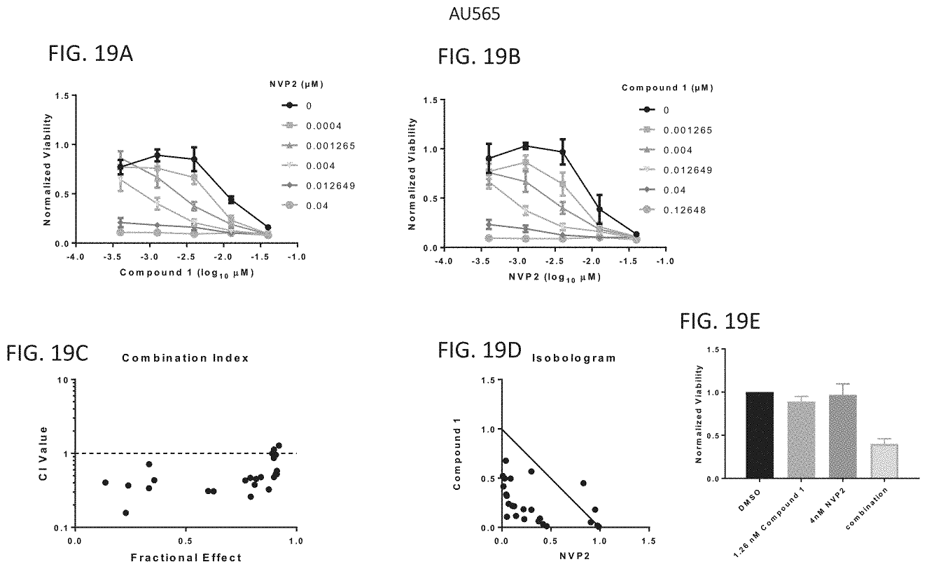

We examined the combined effect of Compound 1 and the CDK9 inhibitor NVP2 on the Her2 amplified, ER⁻/PR⁻ breast cancer cell line AU565 (FIGS. 19A-19D). Synergy was observed for the combination in this cell line.

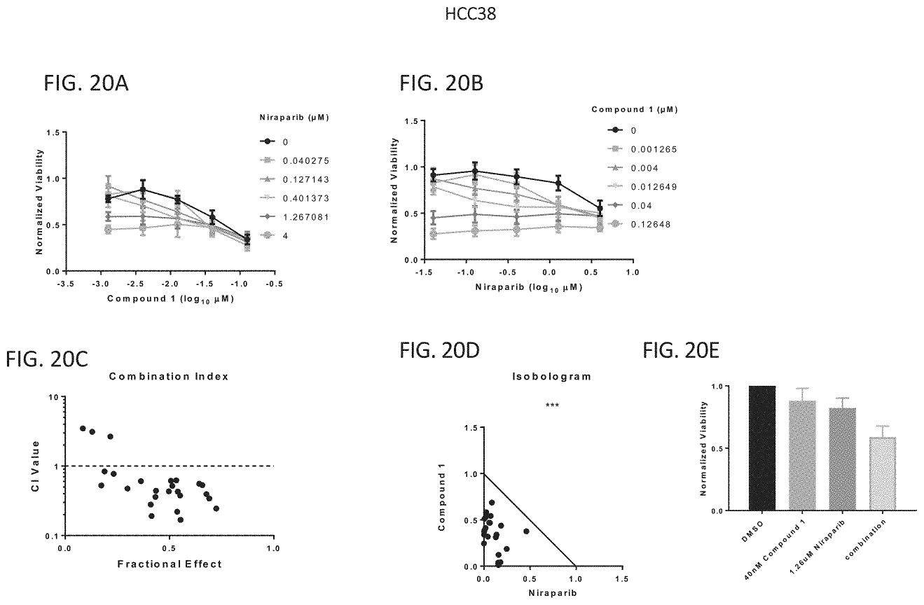

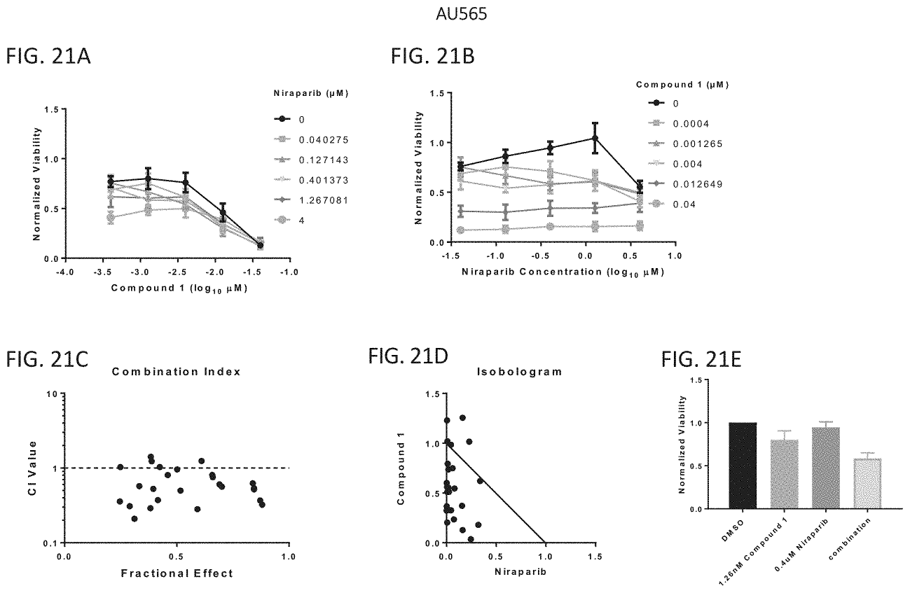

We examined the combined effect of Compound 1 and the PARP inhibitor niraparib on two different breast cancer cell lines—HCC38—a TNBC cell line (FIGS. 20A-20E), and AU565 (FIGS. 21A-21E). Synergy was observed for this combination in both cell lines.

In addition, we compared the ability of JQ1 alone, Compound 1 alone, and a combination of JQ1 and Compound 1 to modify expression of various genes in two breast cancer cell lines. Two triple-negative breast cancer cell lines (HCC70 and MDA-MB468) were tested. The cell lines were treated with JQ1, Compound 1, the JQ1/Compound 1 combination, or DMSO for four hours, after which gene expression profiling via RNA-seq was performed. JQ1 was administered to the cells at a 125 nM final concentration in both single agent and combination experiments. Compound 1 was administered to the cells at a 25 nM final concentration in both single agent and combination experiments. Each experiment was performed in triplicate. One replicate of MDA-MB-468 treated with single-agent JQ1 was excluded from further analysis due to quality.

We employed DESeq2 (Love et al., Genome Biology, 15(12):550, 2014) to identify changes in gene expression due to the JQ1, Compound 1, and JQ1-Compound 1 combination treatment compared to the control DMSO condition based on the RNA-seq data. Genes resulting in a p-value below 0.01 and an absolute logFoldChange above 0.5 were considered to be differentially expressed. In both the HCC70 and MDA-MB468 cell-lines, the combination Compounds 1/JQ1 treatment produced more significantly downregulated genes than single-agent Compound 1 or JQ1 treatments. We identified 3570 significantly downregulated genes from the combination JQ1/Compound 1 treatment in HCC70, while JQ1 alone resulted in 2874 downregulated genes and the individual Compound 1 treatment resulted in 1782 downregulated genes. Similarly, 2414 genes were downregulated in the combination treatment in the MDA-MB468 cell-line, while only 558 and 764 genes were downregulated given the JQ1 and Compound 1 single agent treatments, respectively. A large fraction of these significantly downregulated genes is unique to the combination treatment. 798 of the 3570 downregulated genes following combination treatment in the HCC70 cell-line were not significantly downregulated in either of the individual treatment conditions. The same is true for 1459 of 2414 genes in MDA-MB468.

To identify the genes whose expression is impacted synergistically by the drug combination, we modeled the effect of each drug treatment on expression using a linear model. The expression of each gene after the combination treatment can be thought of as the combined impact of Compound 1, JQ1, and any synergistic interaction between the drug treatments on baseline gene expression. A linear model used to describe this relationship could therefore be represented as:

Gene Expression after combination treatment=Baseline Expression+Compound 1 effect+JQ1 effect+Combination effect (Synergistic Impact)

By fitting the expression data for each gene using this model, we were able to evaluate the effect of each treatment, and identify cases where a synergistic interaction between Compound 1 and JQ1 impacts final gene expression. After fitting the linear model to each gene, the weight and p-value of each term can be evaluated. The combination term's weight signifies the mean change resulting from a synergetic impact on expression, with a negative weight indicating synergistic downregulation. Its associated p-value represents the probability that the combination term is not relevant to final gene expression. Using this approach, we identified 1806 genes in HCC70 and 2205 genes in MDA-MB468 whose expression change in the combination treatment was synergistic and whose associated p-value was less than a threshold cutoff of 0.01. The expression of these synergistic genes could not be explained by an additive effect of Compound 1 and JQ1 alone. Several key transcription factors implicated in breast cancer are downregulated and demonstrate synergy under the combination treatment, including GATA3 (Byrne et al., Histopathology, 2017), FOXC1 (Johnson et al., Oncotarget, 7(46):75729, 2016), and TGIF1 (Zhang et al., Cancer Cell, 27(4):547-650, 2015).

The tables below show how each treatment affected expression of the three genes.

TABLE 5
Effect of JQ1, Compound 1 or a Combination Thereof of Expression
of Certain Genes in Breast Cancer Cell Lines.

| | | | | | Average |
|---|---|---|---|---|---|
| | | | Average | | Expression |
| | | Average | Expression | Average | (TPM) |
| | | Expression | (TPM) | Expression | Compound 1 + |
| Gene | Cell Line | (TPM) DMSO | Compound 1 | (TPM) JQ1 | JQ1 |
| GATA3 | HCC70 | 32.685 | 22.404 | 29.574 | 13.098 |
| | MDA-MB468 | 34.244 | 21.773 | 32.491 | 11.237 |
| FOXC1 | HCC70 | 40.481 | 25.768 | 40.529 | 15.774 |
| | MDA-MB468 | 33.731 | 25.556 | 37.112 | 14.953 |
| TGIF1 | HCC70 | 62.523 | 68.293 | 52.509 | 30.868 |
| | MDA-MB468 | 145.88 | 134.08 | 151.60 | 65.215 |

TABLE 6
Linear Model Coefficients and Significance of
Synergy on Certain Genes for a Combination of
Compound 1 and JQ1 in Breast Cancer Cell Lines.

| | | Synergy | Synergy Coefficient |
|---|---|---|---|
| Gene | Cell Line | Coefficient | Significance |
| GATA3 | HCC70 | −6.1974 | 1.475 × 10⁻³ |
| | MDA-MB468 | −8.7823 | 7.493 × 10⁻³ |
| FOXC1 | HCC70 | −10.042 | 9.188 × 10⁻⁵ |
| | MDA-MB468 | −13.985 | 3.317 × 10⁻³ |
| TGIF1 | HCC70 | −27.411 | 7.978 × 10⁻⁶ |
| | MDA-MB468 | −74.591 | 6.666 × 10⁻⁴ |

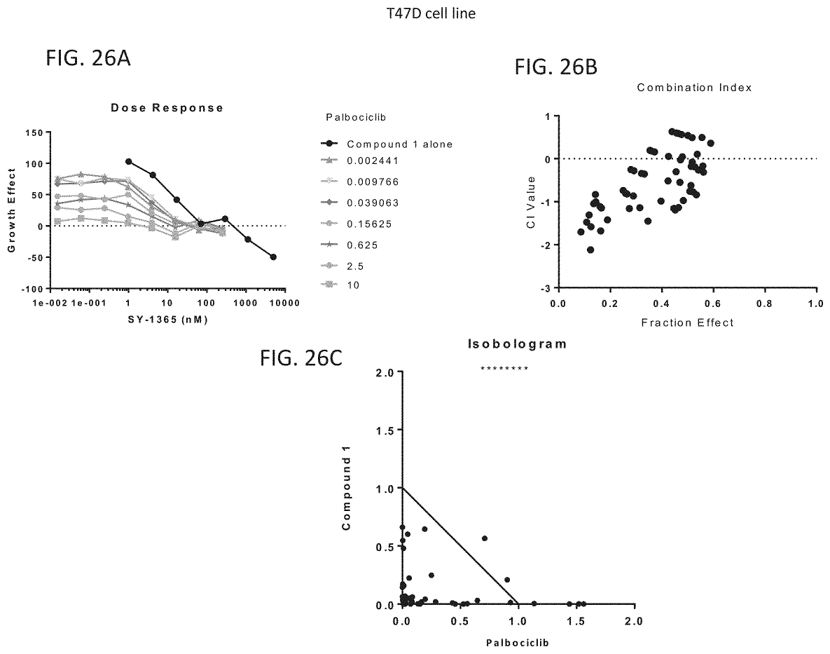

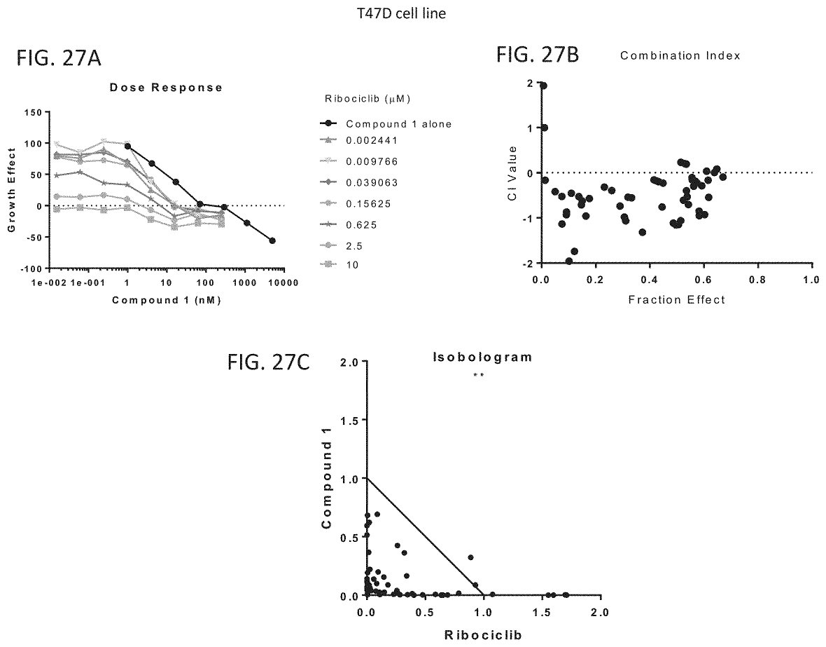

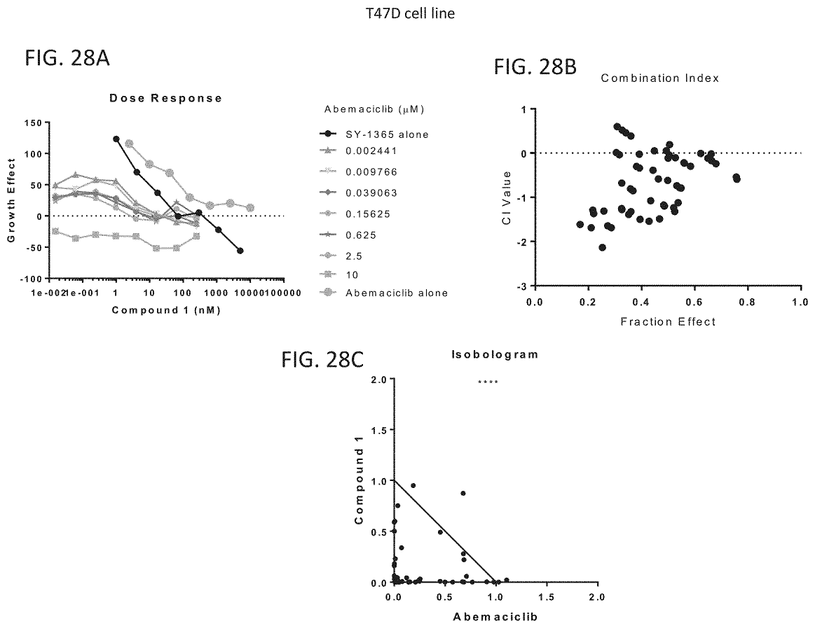

We also examined the combined effect of Compound 1 and three different CDK4/6 inhibitors on the ER⁺ breast cancer cell line T47D. Synergy was observed for all three CDK4/6 inhibitors in combination with Compound 1 (palbociclib, FIGS. 26A-26C; ribociclib, FIGS. 27A-27C; and abemaciclib, FIGS. 28A-28C).

We also examined the combined effect of Compound 1 and the BET inhibitor JQ1 on Ewing's Sarcoma cell lines (SKES, RDES, A673) as well as one osteosarcoma line (Saos2). Cells were grown to 70% confluency in their media of preference based on the manufacturer's recommendations. On the day of assay, cells were lifted and counted using the Countess II FL (Life Technologies). Using a Biotek EL406, 50 μL of preferred cell media containing 30,000 cells/ml was distributed into black 384-well Nunc plates (Thermo) and allowed to adhere overnight prior to compound addition. Compound arrays were distributed to 384 well assay plates using Synergy Plate Format with an HP D300e Digital Dispenser (HP). Compound 1 and JQ1 were dissolved in DMSO to make a stock solution which allowed for accurate dispensing. Compounds were plated in each quadrant of a 384 well plate in quadruplicate. Each quadrant contained test wells with combination of Compound 1 and JQ1 as well as single agent columns, and vehicle wells. After addition of compound, cell plates were incubated for 3 days in a 37° C. incubator. Cell viability was evaluated using ATPlite (Perkin Elmer) following manufacturer protocols. Data was analyzed in CalcuSyn utilizing the median effect principle of presented by Chou-Talalay and visualized using GraphPad Prism Software. Key parameters assessed were combination index and dose reduction index.

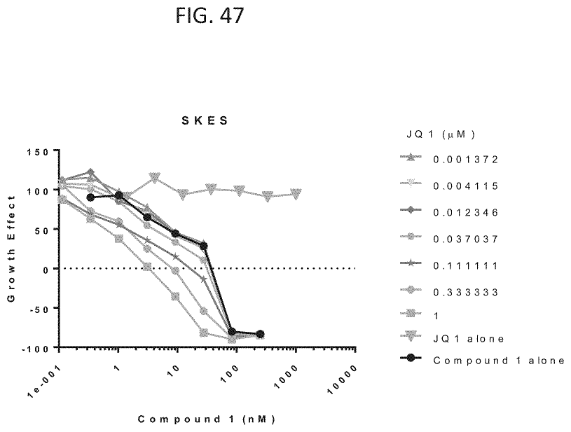

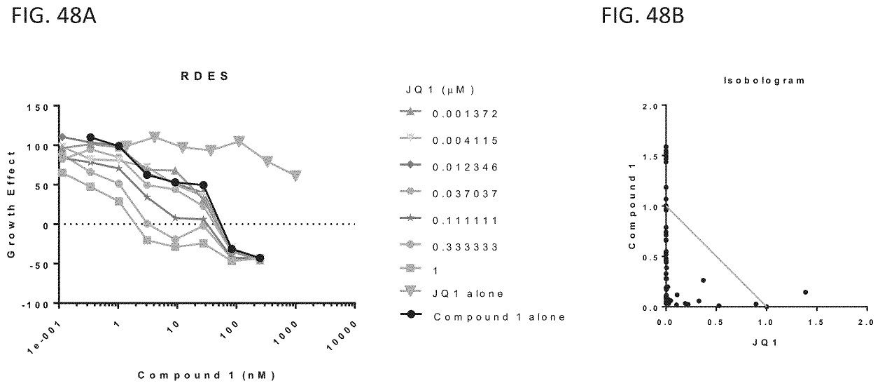

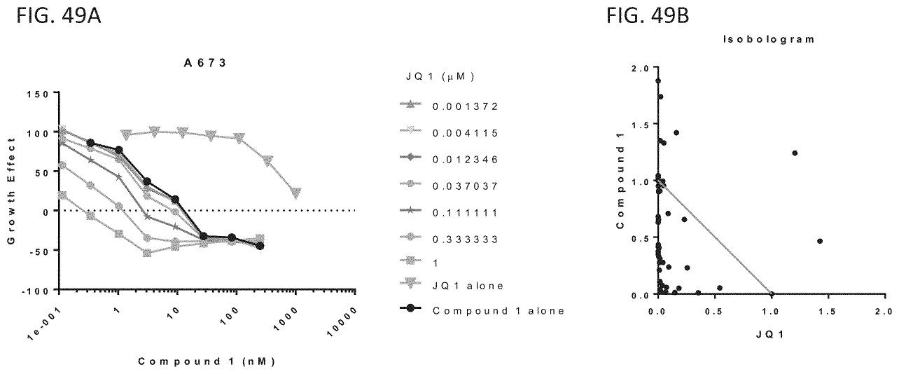

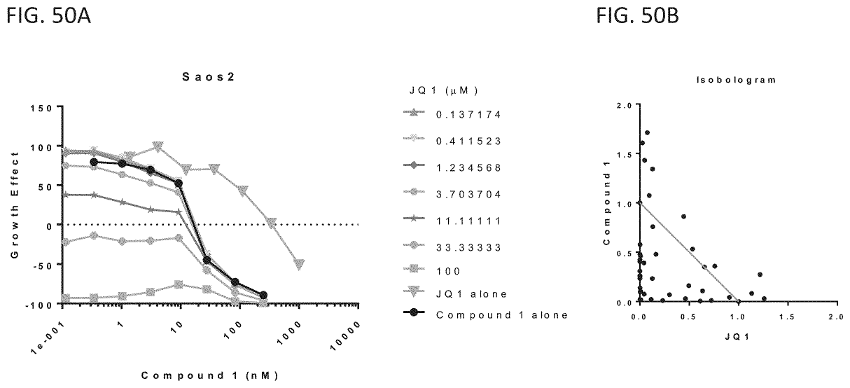

Synergy was observed for JQ1 in combination with Compound 1 for all cell lines (SKES, FIG. 47; RDES, FIGS. 48A-48B; A673, FIGS. 49A-49B; Saos2, FIGS. 50A-50B).

### Example 5. Inhibition of TNBC Cell Line Growth by Compound 1 Correlates with Low BCLXL Expression Levels

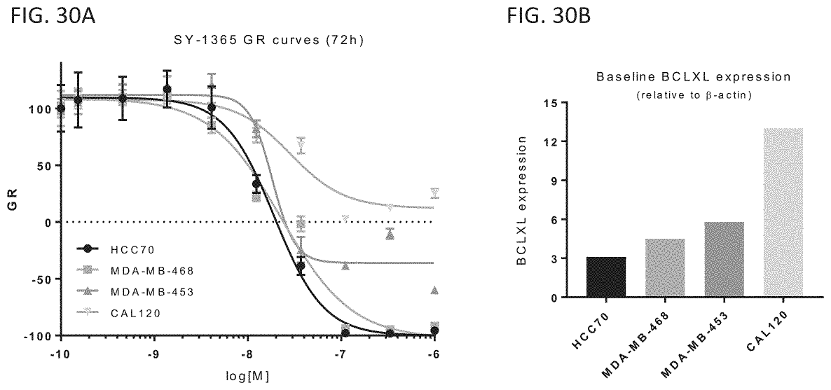

To determine the effect of Compound 1 on the growth of TNBC cancer cells, four different human TNBC cell lines were used—HCC70, MDA-MB-468, MDA-MB-453 and CAL120. Cells from each cell line were plated separately at 50,000 cells/mL (100 uL per well) in a black-walled 96 well plate and allowed to adhere overnight. In parallel, cells were plated in a separate day 0 plate to measure the number of cells present upon compound addition. The next day, Compound 1 was added to the wells with an HP 300e compound dispenser in a 10-point serial dilution and cells incubated for 72 hours. On the same day Cell Titer Glo 2.0 reagent was added to the day 0 plate, and the luminescence measured with an Envision plate reader per manufacture protocol. After 72 hours Cell Titer Glo 2.0 reagent was added to the plates and the luminescence measured. GR curves were calculated as follows:

$$GR ( c ) = {2}^{\frac{{log}_{2} ( x ( c ) / {x}_{0} )}{{log}_{2} ( {x}_{ctrl} / {x}_{0} )}} - 1 ,$$

wherein x(c) is the Compound 1 treatment luminescence); x₀ is the average Day 0 luminescence); and x_{ctrl} is the average DMSO treatment luminescence, and graphed using Graph Pad Prism. A GR value of 1 indicates no growth inhibition; GR values between 0 and 1 indicate partial growth inhibition; a GR value of 0 indicates cytostasis (no change from baseline); GR values less than 0 indicate cytotoxicity (cell number less than baseline); a GR value of −1 indicates complete cell loss. FIG. 30A shows the results of this experiment, with Compound 1 demonstrating almost complete inhibition of growth of both HCC70 and MDA-MB-468 at concentrations greater than 100 nM.

To determine the effect of Compound 1 on the expression of BCL-XL, BCL-2, and MCL1, the same four triple negative breast cancer (TNBC) cell lines (HCC70, MDA-MB-468, MDA-MB-453 and CAL120) were seeded in a six-well plate at a density of 1×10⁶ cells/well and allowed to adhere overnight. Cells were then treated with vehicle (DMSO), 50 nM of Compound 1, or 100 nM of Compound 1 for 24 hours. Cells were harvested, placed on ice, and resuspended in RIPA lysis buffer supplemented with protease and phosphatase inhibitors. Clarified lysates were prepared with 4×LSD running buffer and boiled at 95° C. for 5 minutes. Equivalent amounts of sample (10 μg total protein) were run on a 4-12% Bis-Tris gel and transferred to a PDVF membrane for Western blotting using standard protocols. Membranes were blocked with Licor TBS blocking buffer then probed with primary antibodies for BCL-XL, BCL-2, MCL1, and beta-actin. Antibody-probed membranes were visualized using an Odyssey imager. Sensitivity to Compound 1 correlated well with baseline BCLXL expression with HCC70 and MDA-MB-468 showing the lowest levels of BCLXL expression (derived by densitometry analysis of the Western blot shown in FIG. 29A and shown graphically in FIG. 30B).

### Example 6. Xenograft Models of TNBC and Ovarian Cancer

A. HCC70-Derived Xenografts

Subcutaneous HCC70 xenografts were established in BALC/c nude mice at ChemPartner (Shanghai, China). Each mouse was inoculated subcutaneously in the right flank with 5×10⁶ HCC70 cells (ATCC, CAT #: CRL-2315) in 0.2 ml of a 1:1 mixture of base medium and Matrigel. Tumor sizes were measured in two dimensions using a caliper, and the volumes were expressed in mm³ using the formula: V=0.5 a×b² where a and b are the longest and shortest diameters of the tumor, respectively. Compound 1 treatment was started when the average tumor size reached 181 mm³.

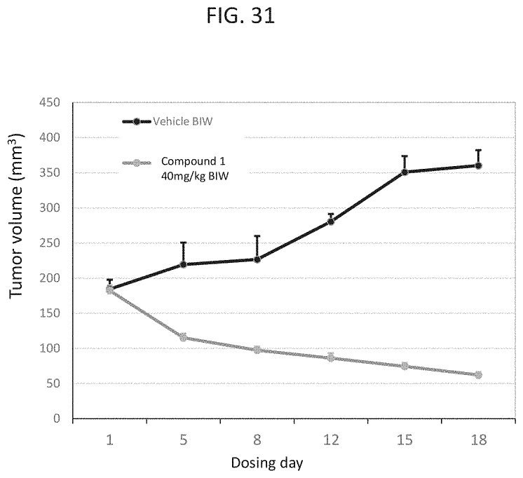

Compound 1 was formulated in w/v 20% Captisol (pH 4-6) and administered by i.v. twice weekly (BIW) at a final dose of 40 mg/kg in a 10 ml/kg volume. Mice in the vehicle arm were given the same dosing schedules, volumes, and formulations, but lacking Compound 1. Tumor volumes were measured twice weekly over the course of the study. As shown in FIG. 31, treatment with Compound 1 caused a reduction in tumor volume, while treatment with vehicle alone resulted in an increase in tumor volume.

For the HCC70 xenograft mice, a sample of the tumor was removed after a single dosing and prepared for Western blotting as described in Example 2. As shown in FIG. 29B, treatment of the HCC70 xenograft mouse with a single dose of 40 mg/kg of Compound 1 reduced MCL1 protein expression, confirming the results obtained with this and other cell lines.

B. TNBC Patient-Derived Xenografts

Patient-derived xenograft (PDX) models from TNBC patients (BR5010, BR5013, BR5015, and BR5023) were established in NOD-SCID mice at Crown Bioscience (San Diego, USA). Cryo vials containing tumor cells were thawed and prepared for injection into mice. Cells were washed in PBS, counted, and resuspended in cold PBS at a concentration of 50,000-100,000 viable cells/100 ul. Cell suspensions were mixed with an equal volume of Cultrex ECM and kept on ice during transport to the vivarium. Cells were prepared for injections by withdrawing ECM-Cell mixture into a chilled slip-tip syringe fitted with a 26G 7/8 (0.5 mm×22 mm) needle. The filled syringes were kept on ice to avoid the solidification of ECM. Each mouse was inoculated subcutaneously in the right flank with 200 uL of the cell suspension. Tumor sizes were measured in two dimensions using a caliper, and the volumes were expressed in mm³ using the formula: V=0.5 a×b², where a and b are the longest and shortest diameters of the tumor, respectively. Compound 1 treatment was started when the average tumor size reached 150-200 mm³.

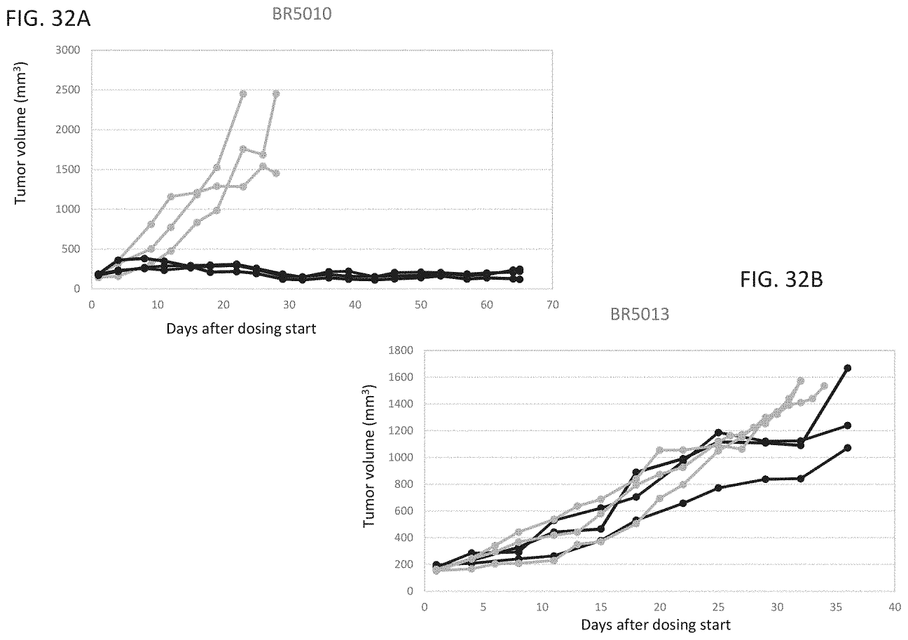

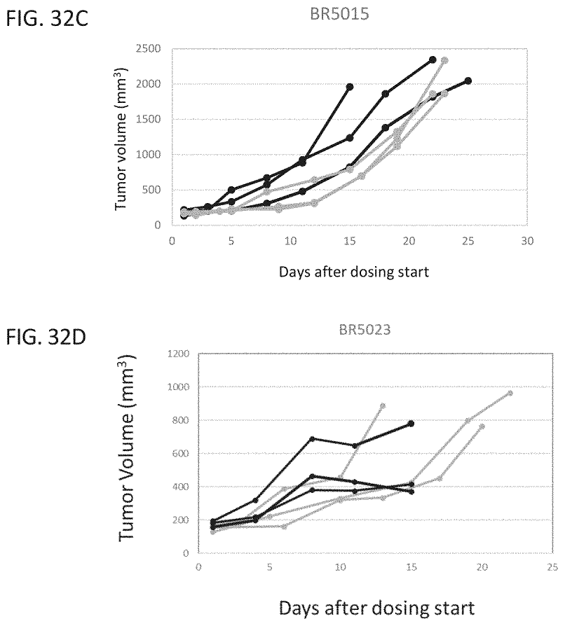

Compound 1 was formulated in w/v 20% Captisol (pH 4-6) and administered by i.v. twice weekly (BIW) at a final dose of 40 mg/kg or 30 mg/kg in a 10 ml/kg volume. Tumor volumes were measured twice weekly over the course of the study. Growth of tumors in Compound 1 treated mice was compared to historical growth of untreated mice for each model. The results of this experiment are shown in FIGS. 32A-32C. As can be seen in FIG. 32A, Compound 1 treatment of BR5010 xenografts consistently inhibited tumor growth, as compared to untreated historical samples. However, Compound 1 had little inhibitory effect on tumor growth in BR5013 (FIG. 32B), BR5015 (FIG. 32C) and BR5023 (FIG. 32D) xenografts.

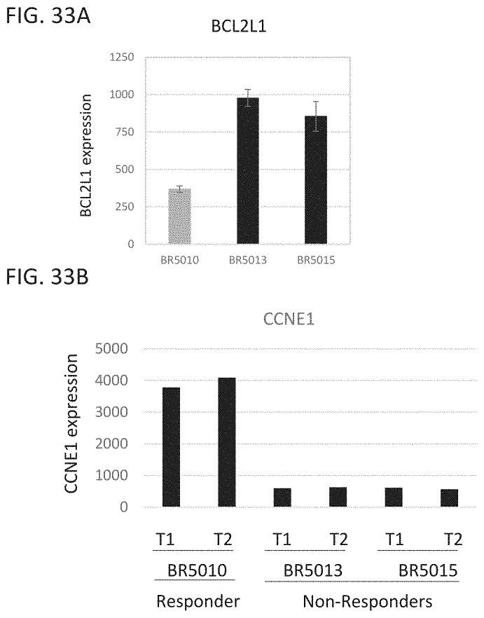

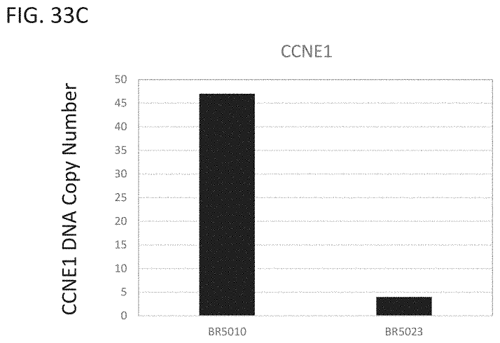

To elucidate the cause of these different responses, we looked at both BCL21 and CCNE1 mRNA levels in each of the xenografts. Approximately 10-20 mg of snap-frozen xenograft tumor tissue samples from PDX models BR5010, BR5013, and BR5015 were pulverized using Cryoprep (Covaris CP02). A total of 750 uL of Trizol reagent (Ambion 15596026) was added to the pulverized sample. Total RNA was extracted using and RNA isolation kit (Invitrogen AM1560), and concentrations of Total RNA were measured with a Nano-drop microvolume spectrophotometer. RNA (100 ng) from each sample was used as input for gene expression assay using the Nanostring nCounter XT technology with the nCounter GX Human Cancer Reference kit. Two independent tumors were assessed per model (T1, T2). Higher CCNE1 expression (FIG. 33B) and lower BCL2L1 expression (FIG. 33A) was observed in the responder line BR5010 compared to the two non-responder lines.

To further evaluate the cause for high CCNE1 expression in BR5010, we evaluated CCNE1 gene copy number in BR5010 and the non-responder cell line BR5023. For DNA extraction, 10-20 mg of pulverized PDX sample was suspended with 180 μL ALT buffer and 20 μL of proteinase K from DNeasy Blood & Tissue Kit (Qiagen 69504). Concentrations of total DNA were evaluated with a Nano-drop microvolume spectrophotometer.

Whole-exome sequencing data from PDX models were analyzed by WuXi NextCODE to determine mutations of interest in each sample and mutations recurring in the cohort. All samples were analyzed using the NextCODE Sequence Miner bioinformatics platform. Mouse read filtering is performed by assessing sequence reads that are “misaligned” to the human reference genome. Genes with the highest number of variants were inspected and analyzed with NCBI BLAST algorithms to confirm bona fide human reads. Germline variants were filtered by removing all variants found in the single nucleotide polymorphism database (dbSNP) with exception of those found in COSMIC. Copy number variations were determined from the filtered bam files using the CNVkit algorithm. Very high CCNE1 gene copy number was observed in the responder line BR5010 whereas relatively normal CCNE1 gene copy number was observed in non-responder line BR5023 (FIG. 33C).

C. Ovarian Cancer Patient-Derived Xenografts

Patient-derived xenograft (PDX) models from ovarian carcinoma patients (OV5387, OV14702, OV14972, OV15398, OV15576, OV15696, OV15612, OV15631) were established in NOD-SCID mice at Crown Bioscience (San Diego, USA). Cryo vials containing tumor cells were thawed and prepared for injection into mice. Cells were washed in PBS, counted, and resuspended in cold PBS at a concentration of 50,000-100,000 viable cells/100 ul. Cell suspensions were mixed with an equal volume of Cultrex ECM and kept on ice during transport to the vivarium. Cells were prepared for injections by withdrawing ECM-Cell mixture into a chilled slip-tip syringe fitted with a 26G 7/8 (0.5 mm×22 mm) needle. The filled syringes were kept on ice to avoid the solidification of ECM. Each mouse was inoculated subcutaneously in the right flank with 200 uL of the cell suspension. Tumor sizes were measured in two dimensions using a caliper, and the volumes were expressed in mm3 using the formula: V=0.5 a×b² where a and b are the longest and shortest diameters of the tumor, respectively. Compound 1 treatment was started when the average tumor size reached 150-200 mm³.

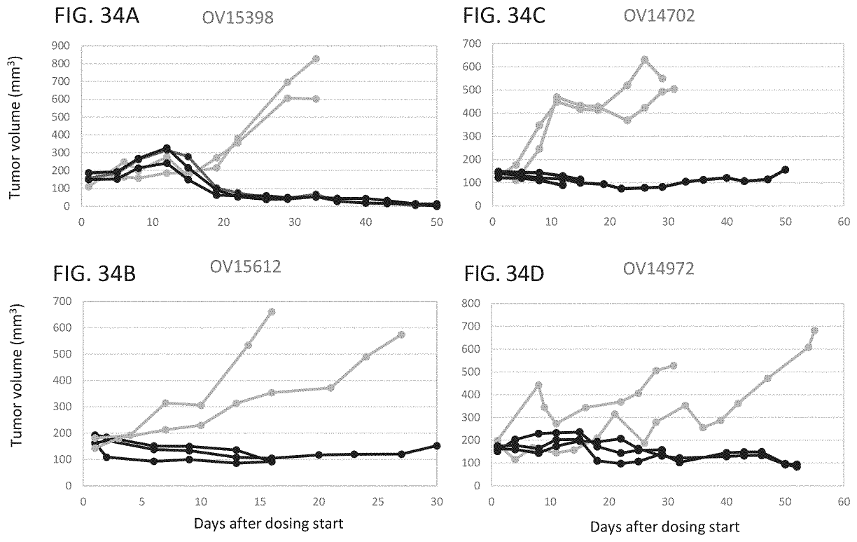

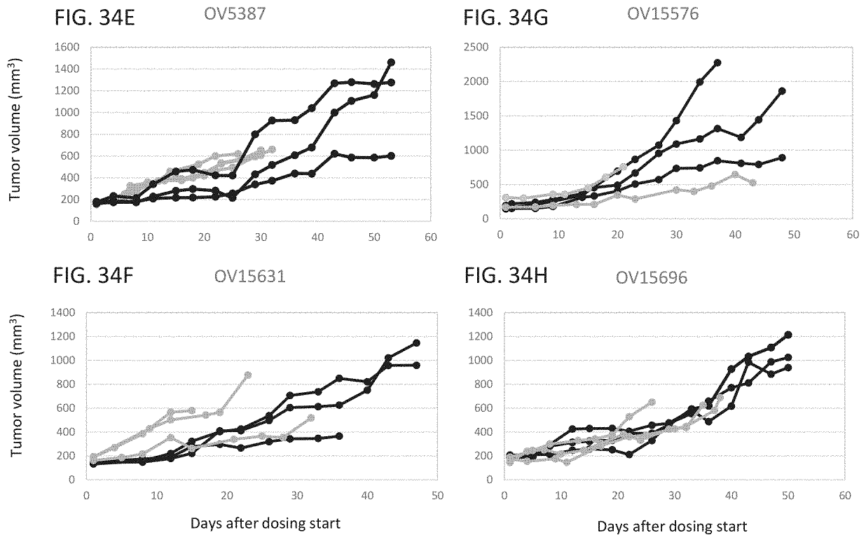

Compound 1 was formulated in w/v 20% Captisol (pH 4-6) and administered by i.v. twice weekly (BIW) at a final dose of 40 mg/kg or 30 mg/kg in a 10 ml/kg volume. Tumor volumes were measured twice weekly over the course of the study. Growth of tumors in Compound 1-treated mice was compared to historical growth of untreated mice for each model. As can be seen from FIGS. 34A-34H, four of the xenografts responded to treatment (FIGS. 34A-34D), while four did not (FIGS. 34E-34H).

In order to elucidate the cause of these different responses, we looked at the level of various proteins from the xenografts. Tumor tissue collected from each of the patient xenografts and was prepared for and subject to Western blot analysis as described in Example 2 using primary antibodies against RB1 (Cell Signaling, CST9309), CCNE1 (Santa Cruz, sc-247), FGFR1 (Cell Signaling, CST9740) or β-ACTIN. For some models, two independent tumors were assessed (T1, T2). As shown in FIG. 35, all responders demonstrated either low RB1 expression or high CCNE1 expression. All 4 non-responders had relatively high RB1 expression; 1/4 non-responders had increased CCNE1 expression (OV15696), although expression levels were lower than those observed in the CCNE1^{HI} responsive model OV15612.

We then preformed RNA extraction to determine gene expression and, DNA extraction to determine gene copy number and mutations, and H3K27Ac ChIP-Seq to determine super enhancers for the various PDX tumors. For RNA extraction, PDX tumors were pulverized using Cryoprep (Covaris CP02). Ten to twenty mg of the pulverized sample were suspended with 750 μL of Trizol reagent (Ambion 15596026). Total RNA was extracted using RNA isolation kit (Invitrogen AM1560). For DNA extraction, 10-20 mg of pulverized PDX sample were suspended with 180 μL ALT buffer and 20 μL of proteinase K from DNeasy Blood & Tissue Kit (Qiagen 69504). Concentrations of total RNA and DNA were evaluated with a Nano-drop microvolume spectrophotometer. One-hundred ng of RNA from each sample were used as input for gene expression assay using Nanostring nCounter XT technology (nCounter GX Human Pan Cancer kit with 30 Custom PLUS gene set). One μg of DNA was sent to WuXiNextCODE for Whole Exome Sequencing (WES-Agilent-V6-100x) for analysis of gene copy number and mutations (single nucleotide variants, InDels).

Samples were analyzed using the NextCODE Sequence Miner bioinformatics platform. Mouse read filtering was performed by assessing sequence reads that were misaligned to the human reference genome. Genes with the highest number of variants were inspected and analyzed with NCBI BLAST algorithms to confirm bona fide human reads. Germline variants were filtered by removing all variants found in the single nucleotide polymorphism database (dbSNP) with exception of those found in COSMIC. Copy number variations were determined from the filtered bam files using the CNVkit algorithm.

For H3K27Ac ChIP-Seq, 10-50 mg of pulverized PDX samples were cross-linked with 1% formaldehyde in PBS for 8 minutes, cross-linking process were quenched by adding 2.5M Glycine. Tissues were then lysed with Lysis buffer LB1 (Boston Bioproducts, CHP-126) and LB2 (Boston Bioproducts, CHP-127) for 10 minutes sequentially. Lysed samples were sonicated using focused ultrasonicator (Covaris, E220), H3k27Ac antibody-conjugated magnetic beads (Abcam ab4729, Invitrogen, 10004D) were added after sonication and incubated overnight in 4° C. Samples were washed and eluted off the beads with Elution buffer (Boston Bioproducts, CHP-153). Reverse cross-link was performed by incubating samples in 65° C. overnight. DNA was precipitated and cleaned using phenol chloroform extraction (Sigma, P3803). Eluted DNA was sequenced and super-enhancer analysis was performed based on the SE-scoring algorithm of M. R. McKeown et al., Cancer Discov, 2017, 7(10), pp. 1136-1153.

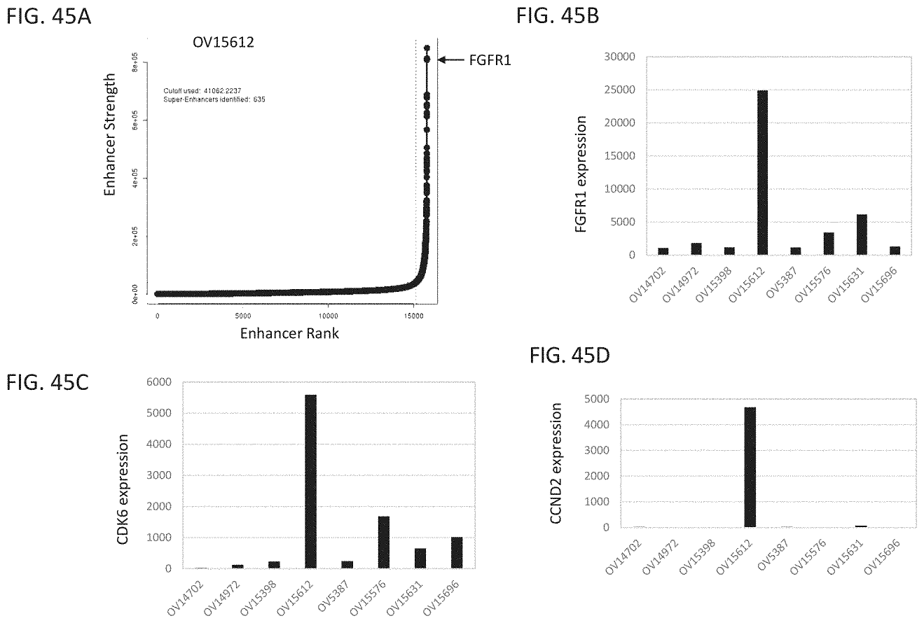

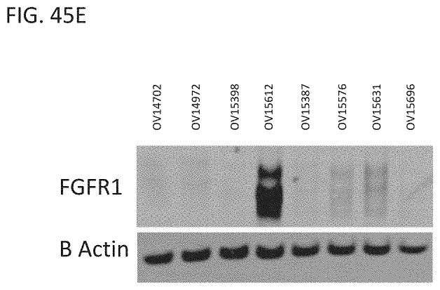

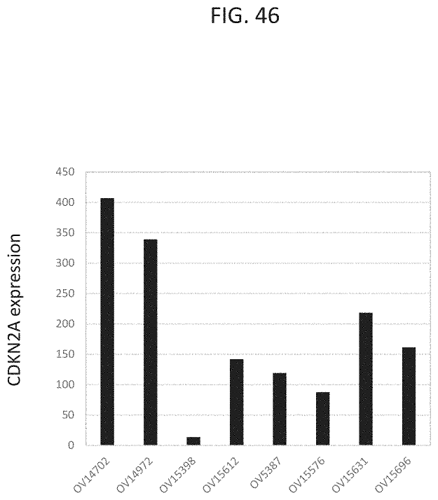

One of the ovarian PDX models that responded to Compound 1 (0V15612) contained a strong super enhancer associated with the FGFR1 gene (FIG. 45A), and highly overexpresses FGFR1, CDK6, and CCND2 mRNA (FIGS. 45B-45D), and FGFR1 protein (FIG. 45E) as compared to other ovarian PDX models we tested. Another ovarian PDX model that responded to Compound 1 (OV15398) had very low expression of the tumor suppressor CDKN2A (P16), a potent inhibitor of CDK4/6 activity compared to other ovarian PDX models (FIG. 46). Yet another ovarian PDX that responded to Compound 1 (OV14702) contained a single copy of the RB1 gene, which had a frameshift mutation (pE204X) indicating it is RB1 null. These results confirm that Compound 1 is effective in cancers that are characterized by molecular alterations that confer CDK 4/6 inhibitor resistance.

### Example 7. A Combination of Venetoclax and Compound 1 Act Synergistically on a BCLXL^{LO} AML Cell Line (KG-1) Xenograft

Subcutaneous KG-1 xenografts were established in CB17 SCID mice at ChemPartner (Shanghai, China). Each mouse was inoculated subcutaneously in the right flank with 5×10⁶ KG-1 cells (ATCC, CAT #: CCL-246) in 0.2 ml of a 1:1 mixture of base medium and Matrigel. Tumor sizes were measured in two dimensions using a caliper, and the volumes were expressed in mm3 using the formula: V=0.5 a×b² where a and b are the longest and shortest diameters of the tumor, respectively. Compound 1 and/or venetoclax treatments were started when the average tumor size reached 200 mm³.

Compound 1 was formulated in 20% w/v Captisol (pH 4-6) and administered by i.v. once weekly (QW) at a final dose of 40 mg/kg in a 10 ml/kg volume. Venetoclax was formulated in 60% Phosal 50 propylene glycol (PG), 30% polyethylene glycol (PEG 400), 10% ethanol, and administered by oral gavage once daily (QD) at a final dose of 50 mg/kg in a 10 ml/kg volume. Mice in the combination arm were given the same dosing schedules, volumes, and formulations for each agent. Mice in the vehicle arm were given the same dosing schedules, volumes, and formulations, but lacking Compound 1 and venetoclax. Tumor volumes were measured twice weekly over the course of the study.

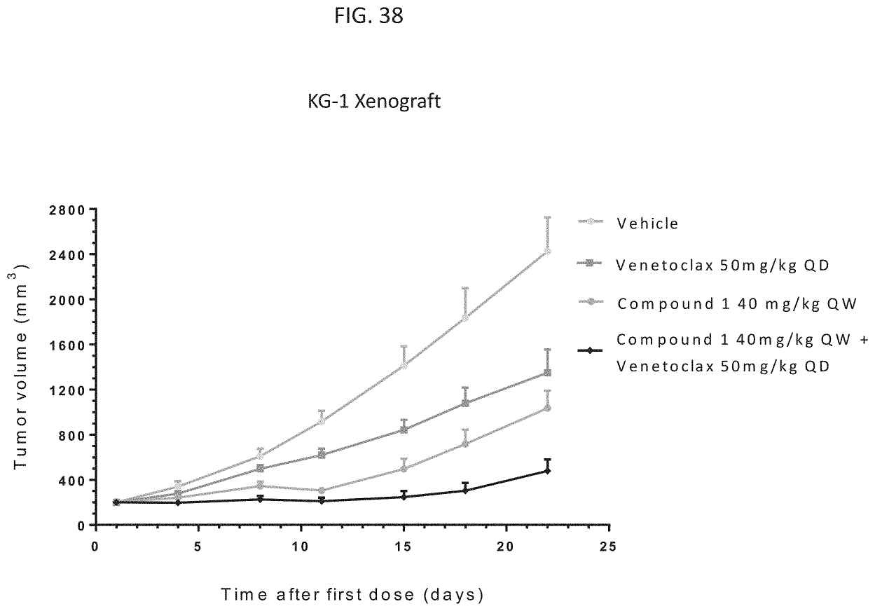

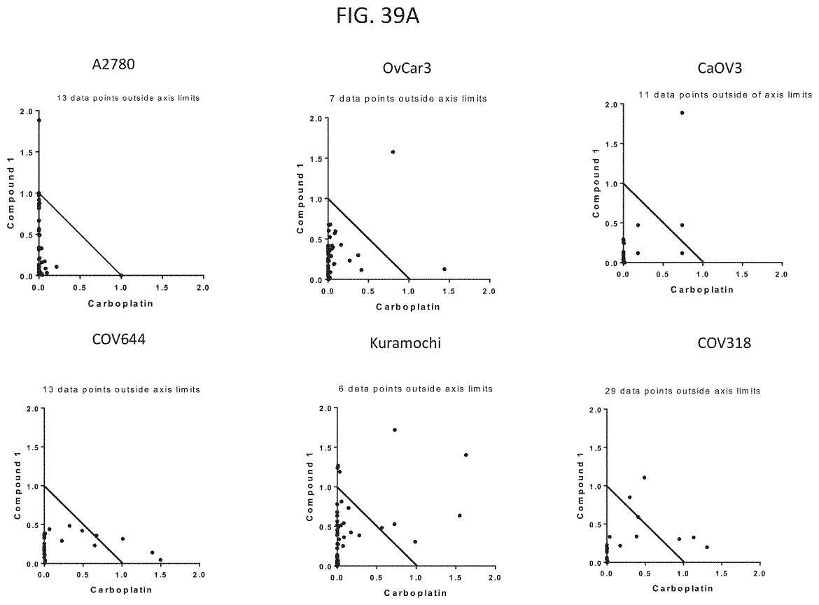

The results of this experiment are shown in FIG. 38, where the combination of venetoclax and Compound 1 had greater inhibitory effect on tumor growth that either agent administered alone.

### Example 8. Combinations of Compound 1 with Carboplatin, Oxaliplatin, Olaparib, or Paclitaxel Act Synergistically on Ovarian Cancer Cell Lines

Ovarian cell lines (OvCar3, CaOV3, COV644, COV318, Kuramochi, OV-90, SKOV3, TOV21G, A2780, ES2, COV504, COV362) were grown to 70% confluency in their media of preference based on the manufacturer recommendations. On the day of assay, cells were lifted, and counted using the Countess II FL (Life Technologies). Using a Biotek EL406, 50 μL of preferred cell media containing 30,000 cells/ml was distributed into black 384-well Nunc plates (Thermo) and allowed to adhere overnight prior to compound addition. Compound arrays were distributed to 384 well assay plates using Synergy Plate Format with an HP D300e Digital Dispenser (HP). Compound 1 and other test agents were dissolved in DMSO to make a stock solution which allowed for accurate dispensing. However, due to solubility and reactivity, the platinum agents carboplatin and oxaliplatin were dissolved in water with addition of 0.03% Tween-20 to allow for dispensing with digital printer. Compounds were plated in each quadrant of a 384 well plate in quadruplicate. Each quadrant contained test wells with combination of Compound 1 and test agent as well as single agent columns, and vehicle wells.

After addition of compound, cell plates were incubated for 3 days in a 37° C. incubator. Cell viability was evaluated using ATPlite (Perkin Elmer) following manufacturer protocols. Data was analyzed in CalcuSyn utilizing the median effect principle of presented by Chou-Talalay and visualized using GraphPad Prism Software. Key parameters assessed were combination index and dose reduction index.

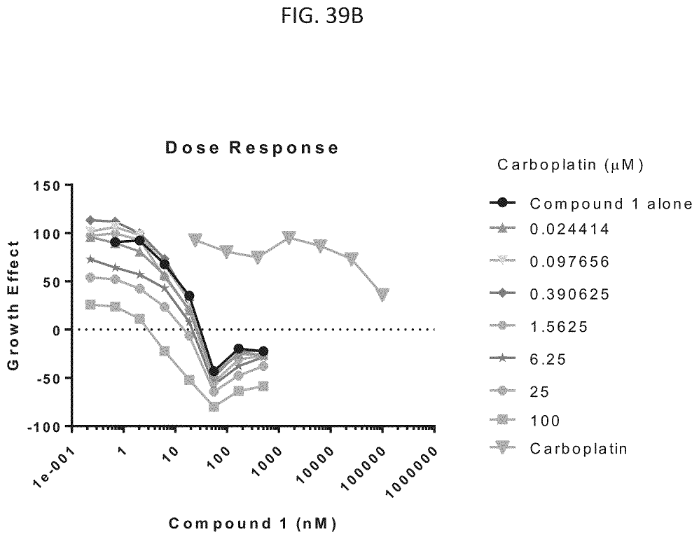

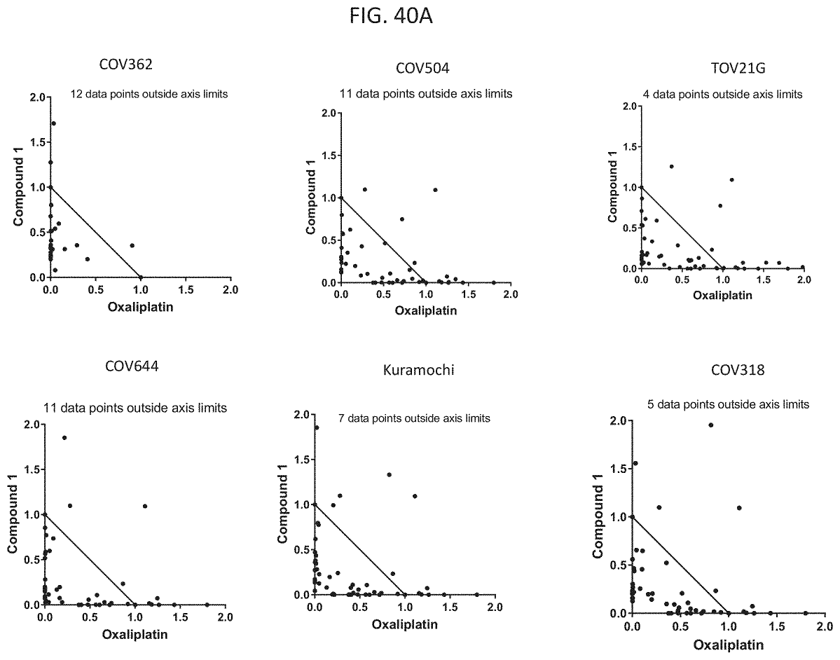

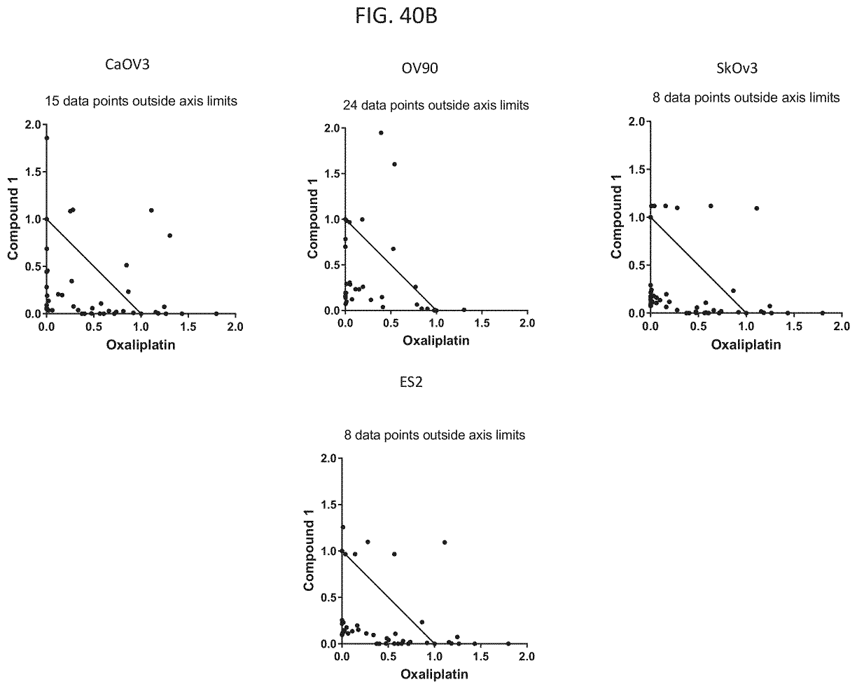

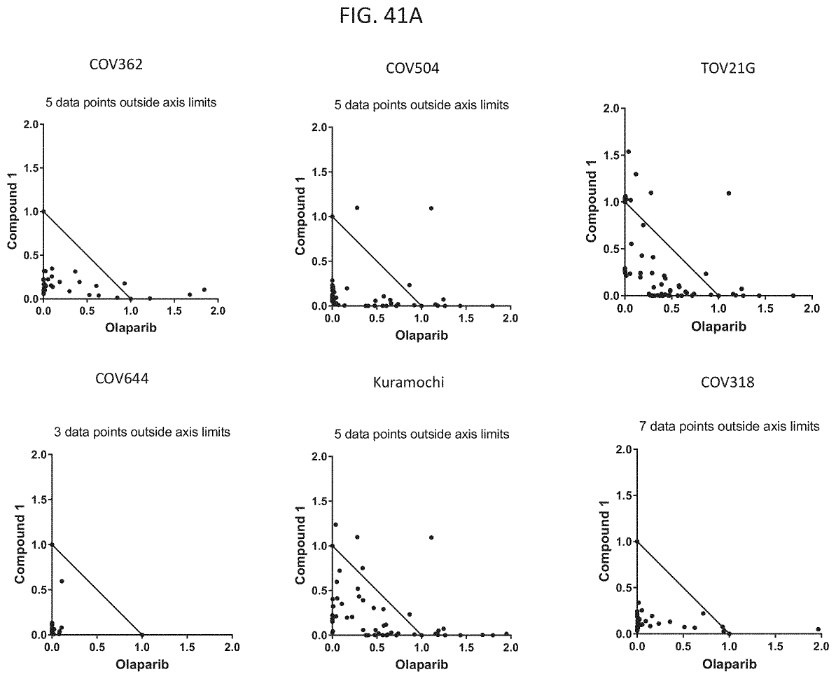

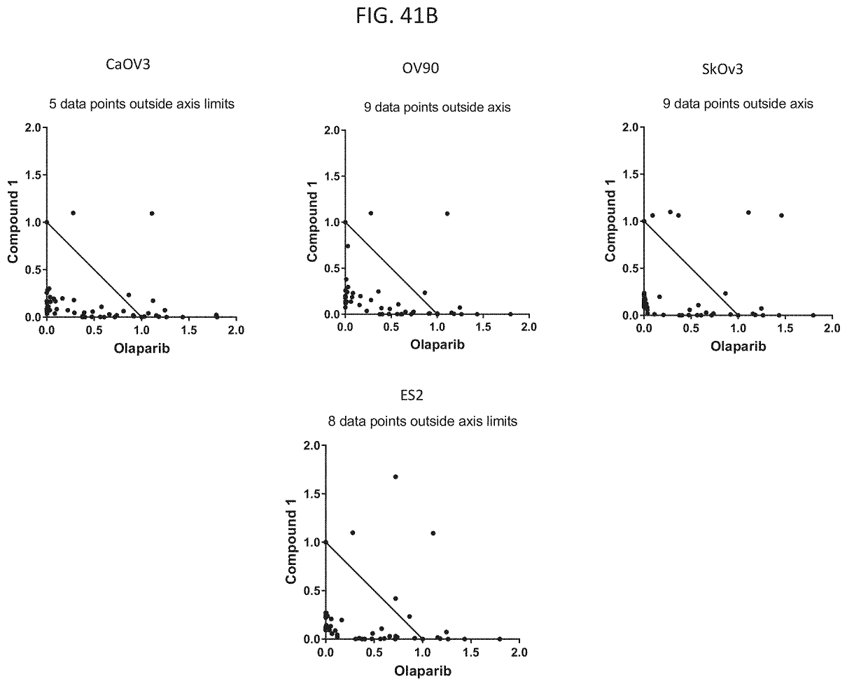

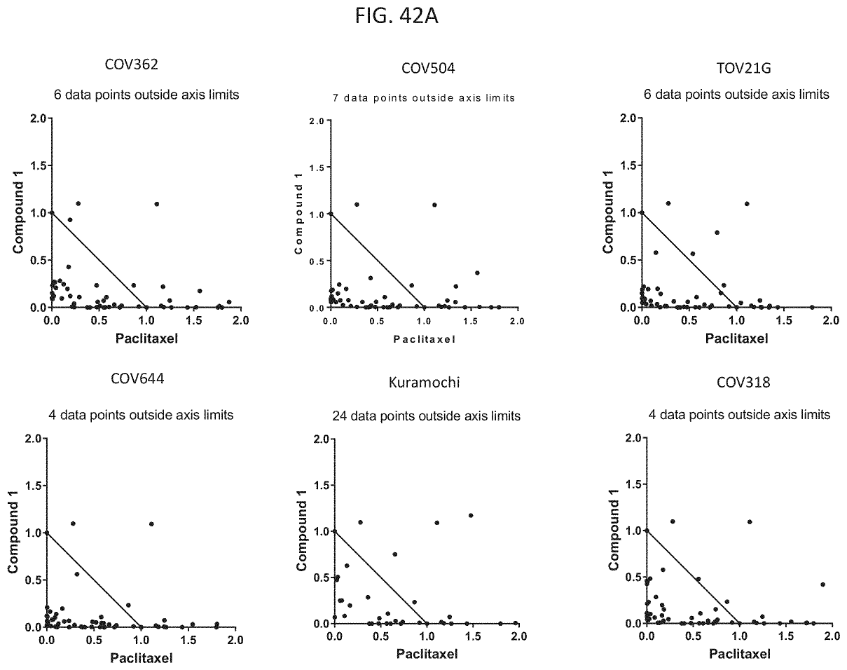

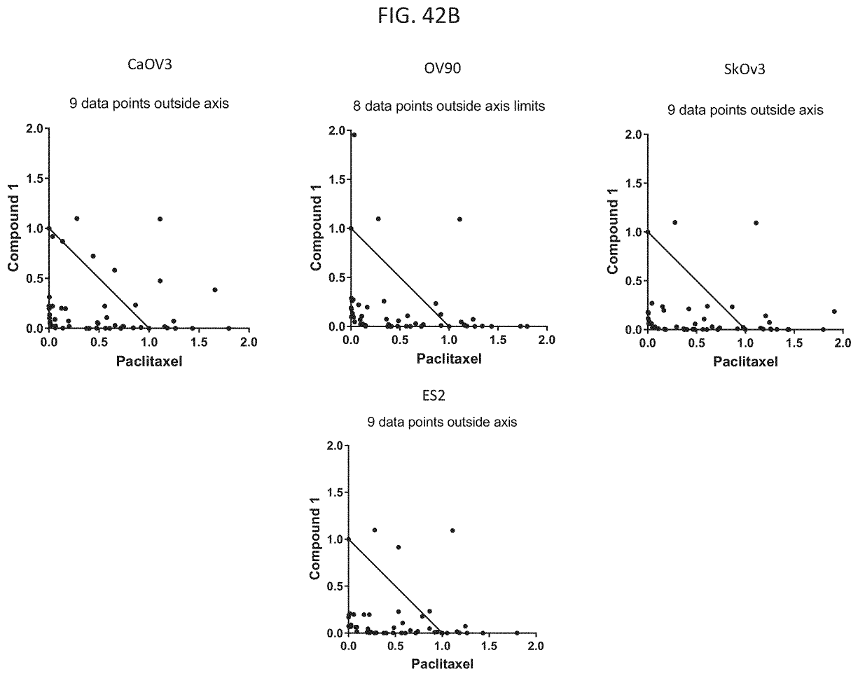

The results of these studies are shown in isobolograms in FIG. 39A (carboplatin), FIGS. 40A-40B (oxaliplatin), FIGS. 41A-41B (olaparib) and FIGS. 42A-42B (paclitaxel). For each agent, synergy is seen in the majority of ovarian cancer cell lines tested. The A2780 cell line is known to be resistant to platinum-based agents, as shown in FIG. 39B. Data from this cell line, combined with the demonstration that Compound 1 causes a decrease in mRNA levels of several genes involved in DNA damage repair (Example 9) and therefore, resistance to platinum-based agent, supports the use of CDK7 inhibitors (e.g., Compound 1) to overcome such resistance.

### Example 9. Compound 1 Causes a Downregulation in mRNA Expression from Genes Involved in DNA Repair in AML, Breast Cancer, and Ovarian Cancer Cell Lines

THP1 is an AML cell line. THP1 cells (1×10⁶/well) were plated in 6-well plates and treated with vehicle (DMSO), 100 nM Compound 1, 25 nM NVP2 (a CDK9 inhibitor), 250 nM JQ1 (a BRD4 inhibitor) or 200 nM Flavopiridol (a pan-CDK inhibitor) for 6 hrs, after which cells were harvested and total RNA (1,000 ng) isolated. RNA levels of the DNA damage repair genes Rad51, CHEK1 and CHEK2 were analyzed by microarray. Experiments were done in triplicates. We performed RMA normalization of the data using the “affy” package from Bioconductor. The command used is shown below and includes background correction, normalization and summarization:

rma_Result <-expresso(raw_data,
bgcorrect.method=“rma”,normalize=TRUE,pmcorrect.method=“pmonly”,
summary.method=“medianpolish”)

Next, we performed loess normalization using only spike-ins to enable comparison of expression values across multiple samples. The command used was:

ma_expr_norm <-normalize.loess(exprs(rma_Result),
subset=grep(“ERCC-”,rownames(exprs(rma_Result))))

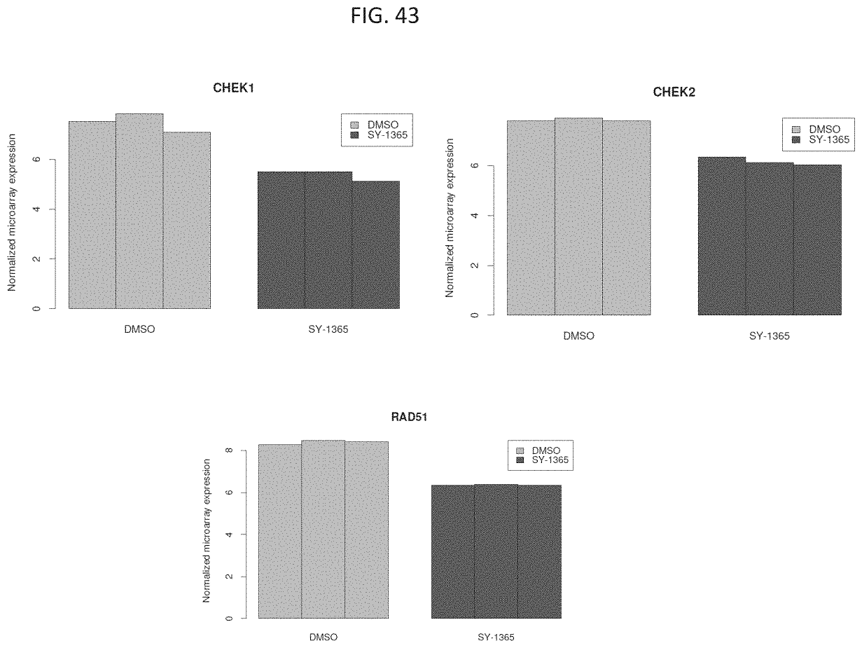

The results are shown in FIG. 43.

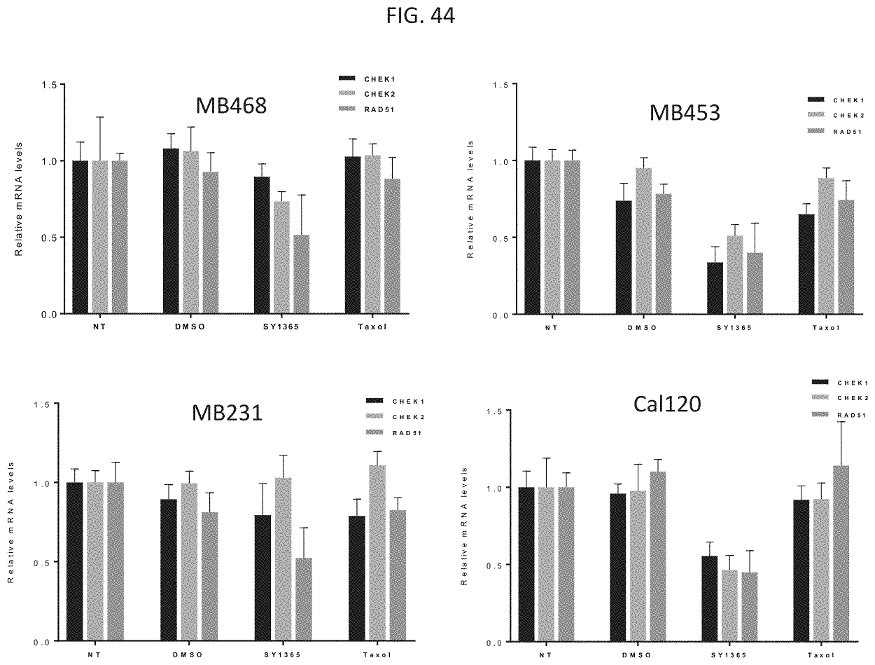

Breast cancer cell lines MDA-MB-468, MDA-MB-231, Ca1120 and MDA-MB-453 were plated at 200,000 cells/well in a 6-well plate the day before treatment. The next day the cells were treated with vehicle, 50 nM Compound 1 or 50 nM Paclitaxel for 6 hrs, after which cells were harvested and total RNA collected using RNeasy mini kit (Qiagen). Total RNA (500 ng) was reverse transcribed using Quantitect Reverse Transcription kit (Qiagen). Quantitative PCR for each of Rad51, CHEK1 and CHEK2 was performed on QuantStudio 6 Flex (Applied Biosystems) using Power SYBR green PCR master mix (Thermo Fisher Scientific) and primers specific for each of those genes. The change in gene expression, relative to housekeeping gene RPL27, was quantified using DDCt method. The results are shown in FIG. 44.

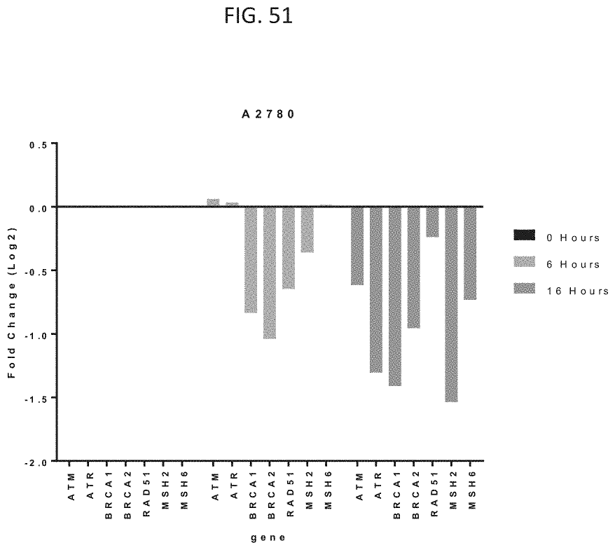

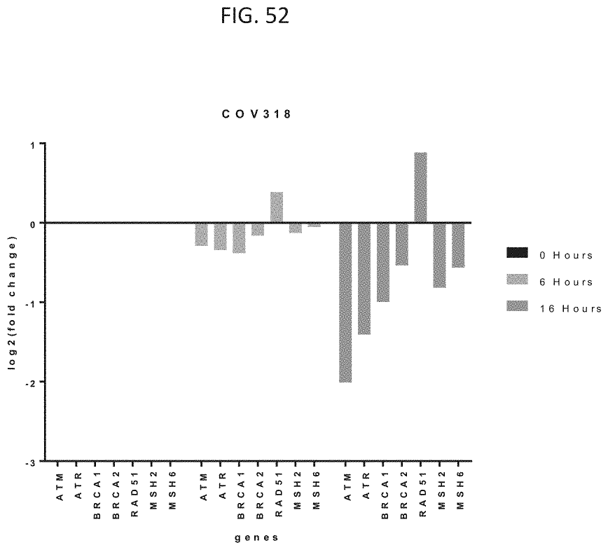

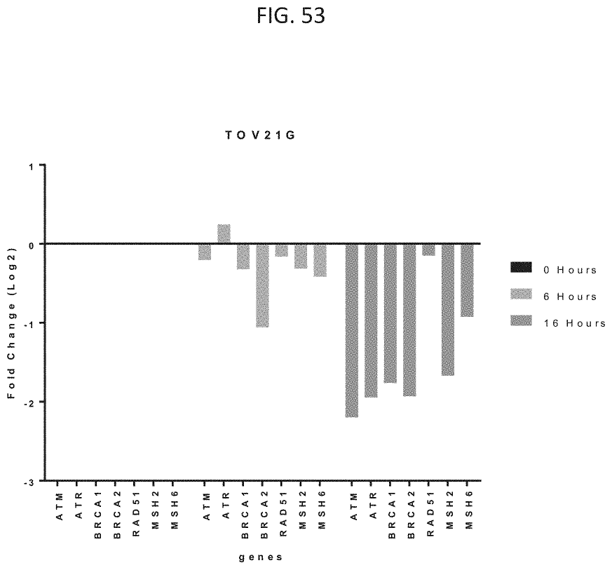

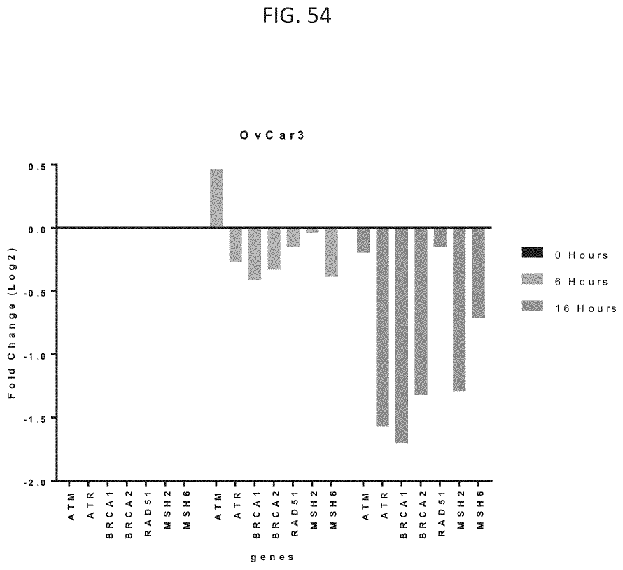

Ovarian Cell lines OvCar3, TOV21G, A2780 and COV318 were plated at 500,000 cells/well in a 6-well plate the day before treatment. The next day the cells were treated with vehicle or 50 nM Compound 1 for 0, 6, or 16 hours after which cells were harvested and total RNA collected using RNeasy mini kit (Qiagen). Changes in mRNA between samples were analyzed with Nanostring™ PanCancer Pathways Panels specifically analyzing genes related to homologous recombination deficiency and carboplatin sensitivity (ie. BRCA1, BRCA2, Rad51, ATM, ATR, MSH2, MSH6). Nanostring signal intensities were first normalized to housekeeping genes across all cell lines, and then normalized to the 0 hour timepoint within a cell line. All genes (except Rad51) were downregulated at 16 hours across all cell lines (A2780, FIG. 51; COV318, FIG. 52; TOV21G, FIG. 53; OvCar3, FIG. 54).

### Example 10: Compound 1 Enhances Carboplatin Tumor Growth Inhibition in Ovarian Cancer Xenografts

Subcutaneous TOV21G xenografts were established in BALB/c nude mice at ChemPartner (Shanghai, China). Each mouse was inoculated subcutaneously in the right flank with 5×10⁶ TOV-21G cells (Human ovarian cancer, ATCC, CRL-11730, 5034683) in 0.2 ml of a 1:1 mixture of base medium and Matrigel. Tumor sizes were measured in two dimensions using a caliper, and the volumes were expressed in mm3 using the formula: V=0.5 a×b² where a and b are the longest and shortest diameters of the tumor, respectively. Compound 1 and/or carboplatin treatments were started when the average tumor size reached 150 mm³.

Subcutaneous OVCAR3 xenografts were established in BALB/c nude mice at Crown Bioscience Inc. (Beijing, China). Each mouse was inoculated subcutaneously in the right flank with 1×10⁷ OVCAR-3 cells (Human ovarian cancer, ATCC HTH-161, NIH:OVCAR-3) in 0.1 ml of a 1:1 mixture of base medium and Matrigel. Tumor sizes were measured in two dimensions using a caliper, and the volumes were expressed in mm3 using the formula: V=0.5 a×b² where a and b are the longest and shortest diameters of the tumor, respectively. Compound 1 and/or carboplatin treatments were started when the average tumor size reached 150 mm³.

Subcutaneous A2780 xenografts were established in BALB/c nude mice at Crown Bioscience Inc. (Beijing, China). Each mouse was inoculated subcutaneously in the right flank with 1×10⁷ A2780 cells (Human ovarian cancer, ECACC 93112519, A2780) in 0.1 ml of a 1:1 mixture of base medium and Matrigel. Tumor sizes were measured in two dimensions using a caliper, and the volumes were expressed in mm3 using the formula: V=0.5 a×b² where a and b are the longest and shortest diameters of the tumor, respectively. Compound 1 and/or carboplatin treatments were started when the average tumor size reached 270 mm³.

Carboplatin was formulated in water and administered by i.p. once weekly (QW) at a final dose of 50 mg/kg in a 10 ml/kg volume. Compound 1 was formulated in 20% w/v Captisol (pH 4-6) and administered by i.v. once weekly (QW) at a final dose of 20 mg/kg in a 10 ml/kg volume for A2780, and a final dose of 30 mg/kg in a 10 ml/kg volume for TOV21G and OVCAR3. SY-1365 was administered 8 hours after carboplatin for each model. Mice in the combination arm were given the same dosing schedules, volumes, and formulations for each agent. Mice in the vehicle arm were given the same dosing schedules, volumes, and formulations, but lacking Compound 1 and carboplatin. Tumor volumes were measured twice weekly over the course of the study.

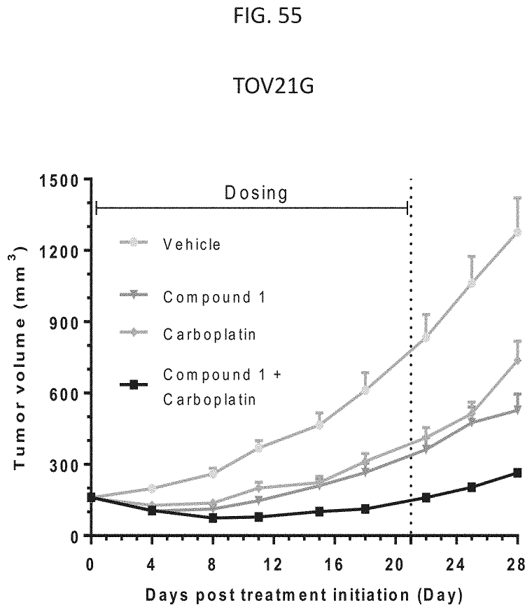

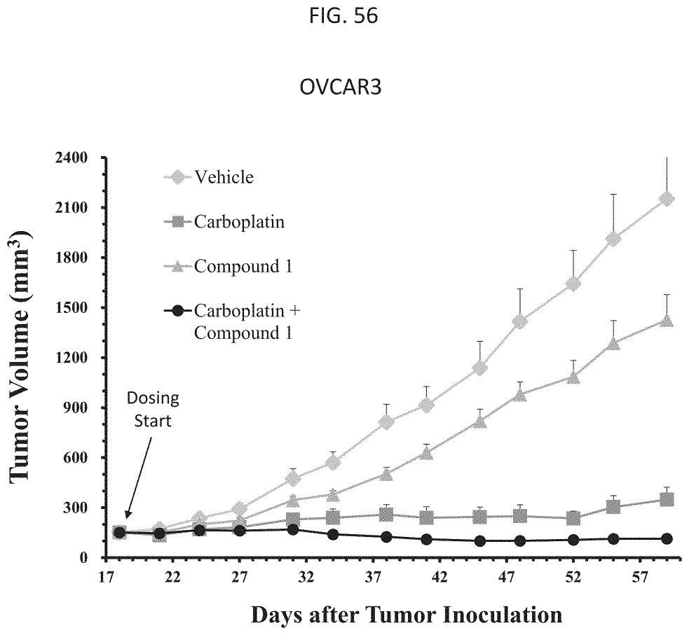

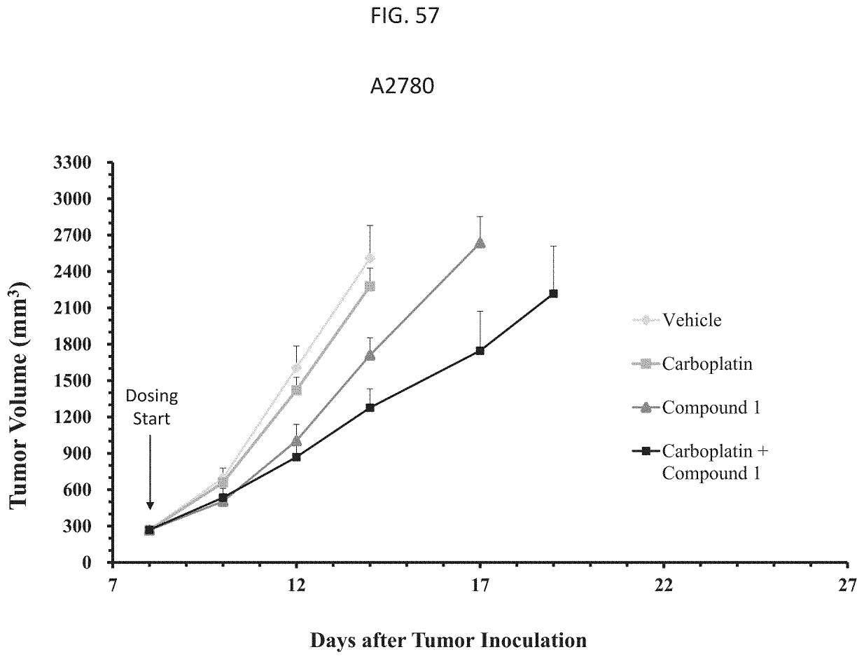

The results of these experiments show that the combination of carboplatin and Compound 1 had greater inhibitory effect on tumor growth than either agent administered alone in all three xenograft models (TOV21G, FIG. 55; OVCAR3, FIG. 56; A2780, FIG. 57).

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “and (v) where ranges are provided, endpoints are included.

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, every possible subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Those skilled in the art will recognize, or be able to ascertain using no more than routine experimentation, that there are many equivalents to the specific embodiments of the disclosure described and claimed herein. Such equivalents are intended to be encompassed by the following claims.

What is claimed is:

1. A therapeutic method comprising administering a compound of the formula 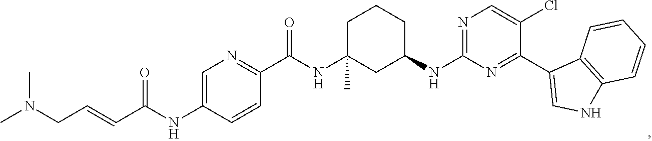 [SMILES: CN(C)C/C=C/C(=O)Nc1ccc(C(=O)N[C@@]2(C)CCC[C@@H](Nc3ncc(Cl)c(-c4c[nH]c5ccccc45)n3)C2)nc1]
or a pharmaceutically acceptable salt thereof, to a patient who has cancer and who is identified as:
(a) having a level of B-cell lymphoma-extra large (BCLXL) mRNA in the cancer equal to or below a pre-determined threshold; and/or
(b) having in at least one of the genes involved in the RB-E2F pathway an alteration in the DNA, an epigenetic alteration, or an alteration in the level of expression of mRNA or protein; and/or
(c) being treated with a platinum-based therapeutic agent or whose cancer has developed resistance to a platinum-based therapeutic agent; and/or
(d) having become or at risk of becoming resistant to treatment with a CDK4/6 inhibitor when used alone or in combination with one or more of an aromatase inhibitor, a selective estrogen receptor modulator or a selective estrogen receptor degrader.

2. The therapeutic method of claim 1, wherein the cancer is a triple negative breast cancer (TNBC), ovarian cancer, non-small cell lung cancer, or acute myeloid leukemia (AML) and the patient has been selected by virtue of having a level of BCLXL mRNA in the cancer equal to or below the pre-determined threshold level.

3. The therapeutic method of claim 2, wherein the patient has undergone, is presently undergoing, or is intending to undergo treatment with a Bcl-2 inhibitor, such as venetoclax.

4. The therapeutic method of claim 1, wherein the patient is selected by virtue of having one or more of:
(a) a level of CCNE1 gene copy number, mRNA or protein in the cancer equal to or above a pre-determined threshold;
(b) a level of RB1 gene copy number, mRNA or protein in the cancer equal to or below a pre-determined threshold, or an absence of an expressed wild-type RB1 gene;
(c) a level of CDK6 mRNA equal to or above a pre-determined threshold level;
(d) a level of CCND2 mRNA equal to or above a pre-determined threshold level; or
(e) a level of CDKN2A mRNA equal to or below a pre-determined threshold level.

5. The therapeutic method of claim 4, wherein the patient is selected by virtue of having a level of CCNE1 gene copy number, mRNA or protein in the cancer equal to or above a pre-determined threshold; a level of RB1 gene copy number, mRNA or protein in the cancer equal to or below a pre-determined threshold; or an absence of an expressed wild-type RB1 gene.

6. The therapeutic method of claim 4, wherein the patient is suffering from ovarian cancer, breast cancer, triple-negative breast cancer, or hormone receptor-positive breast cancer.

7. The therapeutic method of claim 6, wherein the patient has undergone, is presently undergoing, or is intending to undergo treatment with a selective estrogen receptor modulator such as tamoxifen, a selective estrogen receptor degrader such as fulvestrant, and/or a PARP inhibitor, such as olaparib or niraparib.

8. The therapeutic method of claim 1, wherein the patient has become resistant to the platinum-based therapeutic agent.

9. The therapeutic method of claim 1, wherein the platinum-based therapeutic agent is carboplatin or oxaliplatin.

10. The therapeutic method of claim 8, wherein the cancer is ovarian cancer.

11. The therapeutic method of claim 1, wherein the patient has undergone, is presently undergoing, or is intending to undergo treatment with a selective estrogen receptor modulator such as tamoxifen, or a selective estrogen receptor degrader such as fulvestrant.

12. A therapeutic method comprising administering an effective amount of Compound 1 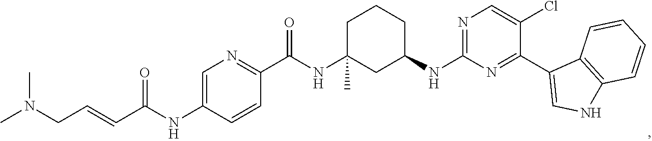 [SMILES: CN(C)C/C=C/C(=O)Nc1ccc(C(=O)N[C@@]2(C)CCC[C@@H](Nc3ncc(Cl)c(-c4c[nH]c5ccccc45)n3)C2)nc1]
or a pharmaceutically acceptable salt thereof, in a combination therapy with an effective amount of a second agent in treating to a patient who has cancer, wherein:
(a) the cancer is TNBC, an estrogen receptor-positive (ER⁺) breast cancer, pancreatic cancer, or a squamous cell cancer of the head or neck and the second agent is a CDK4/6 inhibitor;
(b) the cancer is a breast cancer, or an ovarian cancer and the second agent is a PARP inhibitor;
(c) the cancer is AML, and the second agent is a FLT3 inhibitor;
(d) the cancer is an ovarian cancer and the second agent is a platinum-based anti-cancer agent;
(e) the cancer is TNBC, AML, Ewing's sarcoma, or an osteosarcoma and the second agent is a BET inhibitor; or
(f) the cancer is TNBC, AML, an ovarian cancer, or non-small cell lung cancer and the second agent is a Bcl-2 inhibitor.

13. The therapeutic method of claim 12, wherein the cancer is AML and the second agent is a Bcl-2 inhibitor, such as venetoclax.

14. The therapeutic method of claim 12, wherein the cancer is an epithelial ovarian cancer, a fallopian tube cancer, a primary peritoneal cancer, a triple negative breast cancer or a Her2⁺/ER⁻/PR⁻ breast cancer and the second agent is a PARP inhibitor, such as olaparib or niraparib.

15. The therapeutic method of claim 12, wherein the cancer is an ovarian cancer and the second agent is a platinum-based anti-cancer agent, such as carboplatin or oxaliplatin.

16. A pharmaceutical composition comprising:
(a) an effective amount of Compound 1 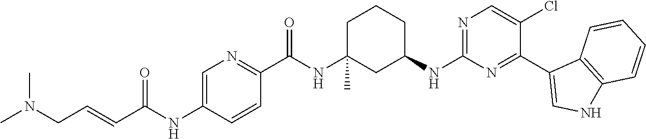 [SMILES: CN(C)C/C=C/C(=O)Nc1ccc(C(=O)N[C@@]2(C)CCC[C@@H](Nc3ncc(Cl)c(-c4c[nH]c5ccccc45)n3)C2)nc1]
or a pharmaceutically acceptable salt thereof;
(b) an effective amount of a second agent selected from a Bcl-2 inhibitor such as venetoclax, a PARP inhibitor such as olaparib or niraparib, a platinum-based anti-cancer agent such as carboplatin or oxaliplatin, a taxane such as paclitaxel, a CDK4/6 inhibitor such as palbociclib, ribociclib, abemaciclib, or trilaciclib, a selective estrogen receptor modulator such as tamoxifen, and a selective estrogen receptor degrader such as fulvestrant; and
(c) a pharmaceutically acceptable carrier.
